(19) United States
(12) Patent Application Publication
(10) Pub. No.: US 2011/0020320 A1
(43) Pub. Date: Jan. 27, 2011

(54) Genetic Variants Contributing to Risk of Prostate Cancer

(75) Inventors: Julius Gudmundsson, Reykjavik (IS); Patrick Sulem, Reykjavik (IS)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: deCODE Genetics ehf., Reykjavik (IS)

(21) Appl. No.: 12/777,113

(22) Filed: May 10, 2010

(30) Foreign Application Priority Data
May 8, 2009 (IS) 8819

(51) Int. Cl. A61K 39/395; G06F 19/00

(52) U.S. Cl. 424/130.1; 702/19

(57) ABSTRACT

It has been discovered that certain genetic markers are associated with risk of prostate cancer. The invention describes diagnostic applications for determining a susceptibilty to prostate cancer using such markers, including methods, uses, kits, and computer applications.

Cancer, the uncontrolled growth of malignant cells, is a major health problem of the modern medical era and is one of the leading causes of death in developed countries. In the United States, one in four deaths is caused by cancer (Jemal, A. et al., CA Cancer J. Clin. 52:23-47 (2002)).

The incidence of prostate cancer has dramatically increased over the last decades and prostate cancer is now a leading cause of death in the United States and Western Europe (Peschel, R. E. and J. W. Colberg, Lancet 4:233-41 (2003); Nelson, W. G. et al., N. Engl. J. Med. 349(4):366-81 (2003)). Prostate cancer is the most frequently diagnosed non-cutaneous malignancy among men in industrialized countries, and in the United States, 1 in 8 men will develop prostate cancer during his life (Simard, J. et al., Endocrinology 143(6):2029-40 (2002)). Although environmental factors, such as dietary factors and lifestyle-related factors, contribute to the risk of prostate cancer, genetic factors have also been shown to play an important role. Indeed, a positive family history is among the strongest epidemiological risk factors for prostate cancer, and twin studies comparing the concordant occurrence of prostate cancer in monozygotic twins have consistently revealed a stronger hereditary component in the risk of prostate cancer than in any other type of cancer (Nelson, W. G. et al., N. Engl. J. Med. 349(4):366-81 (2003); Lichtenstein P. et al., N. Engl. J. Med. 343(2):78-85 (2000)). In addition, an increased risk of prostate cancer is seen in 1^{st} to 5^{th} degree relatives of prostate cancer cases in a nationwide study on the familiality of all cancer cases diagnosed in Iceland from 1955-2003 (Amundadottir et al., PLoS Medicine 1(3):e65 (2004)). The genetic basis for this disease, emphasized by the increased risk among relatives, is further supported by studies of prostate cancer among particular populations: for example, African Americans have among the highest incidence of prostate cancer and mortality rate attributable to this disease: they are 1.6 times as likely to develop prostate cancer and 2.4 times as likely to die from this disease than European Americans (Ries, L. A. G. et al., NIH Pub. No. 99-4649 (1999)).

An average 40% reduction in life expectancy affects males with prostate cancer. If detected early, prior to metastasis and local spread beyond the capsule, prostate cancer can be cured (e.g., using surgery). However, if diagnosed after spread and metastasis from the prostate, prostate cancer is typically a fatal disease with low cure rates. While prostate-specific antigen (PSA)-based screening has aided early diagnosis of prostate cancer, it is neither highly sensitive nor specific (Punglia et al., N Engl J Med. 349(4):335-42 (2003)). This means that a high percentage of false negative and false positive diagnoses are associated with the test. The consequences are both too many instances of missed cancers and unnecessary follow-up biopsies for those without cancer. As many as 65 to 85% of individuals (depending on age) with prostate cancer have a PSA value less than or equal to 4.0 ng/mL, which has traditionally been used as the upper limit for a normal PSA level (Punglia et. al., N Engl J. Med. 349(4):335-42 (2003); Cookston, M. S., Cancer Control 8(2):133-40 (2001); Thompson, I. M. et. al., N Engl J Med. 350:2239-46 (2004)). A significant fraction of those cancers with low PSA levels are scored as Gleason grade 7 or higher, which is a measure of an aggressive prostate cancer.

In addition to the sensitivity problem outlined above, PSA testing also has difficulty with specificity and predicting prognosis. PSA levels can be abnormal in those without prostate cancer. For example, benign prostatic hyperplasia (BPH) is one common cause of a false-positive PSA test. In addition, a variety of non-cancer conditions may elevate serum PSA levels, including urinary retention, prostatitis, vigorous prostate massage and ejaculation.

Furthermore, subsequent confirmation of prostate cancer using needle biopsy in patients with positive PSA levels is difficult if the tumor is too small to see by ultrasound. Multiple random samples are typically taken but diagnosis of prostate cancer may be missed because of the sampling of only small amounts of tissue. Digital rectal examination (DRE) also misses many cancers because only the posterior lobe of the prostate is examined. As early cancers are nonpalpable, cancers detected by DRE may already have spread outside the prostate (Mistry K. J., Am. Board Fam. Pract. 16(2):95-101 (2003)).

Thus, there is clearly a great need for improved diagnostic procedures that would facilitate early-stage prostate cancer detection and prognosis, as well as aid in preventive and curative treatments of the disease that would help to avoid invasive and costly procedures for patients not at significant risk.

Genetic risk is conferred by subtle differences in genes among individuals in a population. Genes differ between individuals most frequently due to single nucleotide polymorphisms (SNP), although other variations are also important. SNP are located on average every 1000 base pairs in the human genome. Accordingly, a typical human gene containing 250,000 base pairs may contain 250 different SNP. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphism in the human genome is caused by insertion, deletion, translocation, or inversion of either short or long stretches of DNA. Genetic polymorphisms conferring disease risk may therefore directly alter the amino acid sequence of proteins, may increase the amount of protein produced from the gene, or may decrease the amount of protein produced by the gene.

As genetic polymorphisms conferring risk of common diseases are uncovered, genetic testing for such risk factors is becoming important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

Although genetic factors are among the strongest epidemiological risk factors for prostate cancer, the search for genetic determinants involved in the disease has been challenging. Studies have revealed that linking candidate genetic markers to prostate cancer has been more difficult than identifying susceptibility genes for other cancers, such as breast, ovary and colon cancer. Several reasons have been proposed for this increased difficulty including: the fact that prostate cancer is often diagnosed at a late age thereby often making it difficult to obtain DNA samples from living affected individuals for more than one generation; the presence within high-risk pedigrees of phenocopies that are associated with a lack of distinguishing features between hereditary and sporadic forms; and the genetic heterogeneity of prostate cancer and the accompanying difficulty of developing appropriate statistical transmission models for this complex disease (Simard, J. et al., Endocrinology 143(6):2029-40 (2002)).

Various genome scans for prostate cancer-susceptibility genes have been conducted and several prostate cancer susceptibility loci have been reported. For example, HPC1 (1q24-q25), PCAP (1q42-q43), HCPX (Xq27-q28), CAPB (1p36), HPC20 (20q13), HPC2/ELAC2 (17p11) and 16q23 have been proposed as prostate cancer susceptibility loci (Simard, J. et al., Endocrinology 143(6):2029-40 (2002); Nwosu, V. et al., Hum. Mol. Genet. 10(20):2313-18 (2001)). In a genome scan conducted by Smith et al., the strongest evidence for linkage was at HPC1, although two-point analysis also revealed a LOD score of ≧1.5 at D4S430 and LOD scores ≧1.0 at several loci, including markers at Xq27-28 (Ostrander E. A. and J. L. Stanford, Am. J. Hum. Genet. 67:1367-75 (2000)). In other genome scans, two-point LOD scores of 1.5 for chromosomes 10q, 12q and 14q using an autosomal dominant model of inheritance, and chromosomes 1q, 8q, 10q and 16p using a recessive model of inheritance, have been reported, as well as nominal evidence for linkage to chr 2q, 12p, 15q, 16q and 16p. A genome scan for prostate cancer predisposition loci using a small set of Utah high risk prostate cancer pedigrees and a set of 300 polymorphic markers provided evidence for linkage to a locus on chromosome 17p (Simard, J. et al., Endocrinology 143(6):2029-40 (2002)). Eight new linkage analyses were published in late 2003, which depicted remarkable heterogeneity. Eleven peaks with LOD scores higher than 2.0 were reported, none of which overlapped (see Actane consortium, Schleutker et al., Wiklund et al., Witte et al., Janer et al., Xu et al., Lange et al., Cunningham et al.; all of which appear in Prostate, vol. 57 (2003)).

As described above, identification of particular genes involved in prostate cancer has been challenging. One gene that has been implicated is RNASEL, which encodes a widely expressed latent endoribonuclease that participates in an interferon-inducible RNA-decay pathway believed to degrade viral and cellular RNA, and has been linked to the HPC locus (Carpten, J. et al., Nat. Genet. 30:181-84 (2002); Casey, G. et al., Nat. Genet. 32(4):581-83 (2002)). Mutations in RNASEL have been associated with increased susceptibility to prostate cancer. For example, in one family, four brothers with prostate cancer carried a disabling mutation in RNASEL, while in another family, four of six brothers with prostate cancer carried a base substitution affecting the initiator methionine codon of RNASEL. Other studies have revealed mutant RNASEL alleles associated with an increased risk of prostate cancer in Finnish men with familial prostate cancer and an Ashkenazi Jewish population (Rokman, A. et al., Am J. Hum. Genet. 70:1299-1304 (2002); Rennert, H. et al., Am J. Hum. Genet. 71:981-84 (2002)). In addition, the Ser217Leu genotype has been proposed to account for approximately 9% of all sporadic cases in Caucasian Americans younger than 65 years (Stanford, J. L., Cancer Epidemiol. Biomarkers Prev. 12(9):876-81 (2003)). In contrast to these positive reports, however, some studies have failed to detect any association between RNASEL alleles with inactivating mutations and prostate cancer (Wang, L. et al., Am. J. Hum. Genet. 71:116-23 (2002); Wiklund, F. et al., Clin. Cancer Res. 10(21):7150-56 (2004); Maier, C. et. al., Br. J. Cancer 92(6):1159-64 (2005)).

The macrophage-scavenger receptor 1 (MSR1) gene, which is located at 8p22, has also been identified as a candidate prostate cancer-susceptibility gene (Xu, J. et al., Nat. Genet. 32:321-25 (2002)). A mutant MSR1 allele was detected in approximately 3% of men with nonhereditary prostate cancer but only 0.4% of unaffected men. However, not all subsequent reports have confirmed these initial findings (see, e.g., Lindmark, F. et al., Prostate 59(2):132-40 (2004); Seppala, E. H. et al., Clin. Cancer Res. 9(14):5252-56 (2003); Wang, L. et al., Nat. Genet. 35(2):128-29 (2003); Miller, D. C. et al., Cancer Res. 63(13):3486-89 (2003)). MSR1 encodes subunits of a macrophage-scavenger receptor that is capable of binding a variety of ligands, including bacterial lipopolysaccharide and lipoteicholic acid, and oxidized high-density lipoprotein and low-density lipoprotein in serum (Nelson, W. G. et al., N. Engl. J. Med. 349(4):366-81 (2003)).

The ELAC2 gene on Chr17p was the first prostate cancer susceptibility gene to be cloned in high risk prostate cancer families from Utah (Tavtigian, S. V., et al., Nat. Genet. 27(2):172-80 (2001)). A frameshift mutation (1641InsG) was found in one pedigree. Three additional missense changes: Ser217Leu; Ala541Thr; and Arg781His, were also found to associate with an increased risk of prostate cancer. The relative risk of prostate cancer in men carrying both Ser217Leu and Ala541Thr was found to be 2.37 in a cohort not selected on the basis of family history of prostate cancer (Rebbeck, T. R., et al., Am. J. Hum. Genet. 67(4):1014-19 (2000)). Another study described a new termination mutation (Glu216X) in one high incidence prostate cancer family (Wang, L., et al., Cancer Res. 61(17):6494-99 (2001)). Other reports have not demonstrated strong association with the three missense mutations, and a recent metaanalysis suggests that the familial risk associated with these mutations is more moderate than was indicated in initial reports (Vesprini, D., et al., Am. J. Hum. Genet. 68(4):912-17 (2001); Shea, P. R., et al., Hum. Genet. 111(4-5):398-400 (2002); Suarez, B. K., et al., Cancer Res. 61(13):4982-84 (2001); Severi, G., et al., J. Natl. Cancer Inst. 95(11):818-24 (2003); Fujiwara, H., et al., J. Hum. Genet. 47(12):641-48 (2002); Camp, N. J., et al., Am. J. Hum. Genet. 71(6):1475-78 (2002)).

Polymorphic variants of genes involved in androgen action (e.g., the androgen receptor (AR) gene, the cytochrome P-450c17 (CYP17) gene, and the steroid-5-α-reductase type II (SRD5A2) gene), have also been implicated in increased risk of prostate cancer (Nelson, W. G. et al., N. Engl. J. Med. 349(4):366-81 (2003)). With respect to AR, which encodes the androgen receptor, several genetic epidemiological studies have shown a correlation between an increased risk of prostate cancer and the presence of short androgen-receptor polyglutamine repeats, while other studies have failed to detect such a correlation. Linkage data has also implicated an allelic form of CYP17, an enzyme that catalyzes key reactions in sex-steroid biosynthesis, with prostate cancer (Chang, B. et al., Int. J. Cancer 95:354-59 (2001)). Allelic variants of SRD5A2, which encodes the predominant isozyme of 5-α-reductase in the prostate and functions to convert testosterone to the more potent dihydrotestosterone, have been associated with an increased risk of prostate cancer and with a poor prognosis for men with prostate cancer (Makridakis, N. M. et al., Lancet 354:975-78 (1999); Nam, R. K. et al., Urology 57:199-204 (2001)).

Despite the effort of many groups around the world, the genes that account for a substantial fraction of prostate cancer risk have not been identified. Although twin studies have implied that genetic factors are likely to be prominent in prostate cancer, relatively few genes have been identified as being associated with an increased risk for prostate cancer, and these genes account for only a low percentage of cases. Thus, it could be that the majority of genetic risk factors for prostate cancer remain to be found. It is likely that these genetic risk factors will include a relatively high number of low-to-medium risk genetic variants but indeed be responsible for a substantial fraction of prostate cancer, and their identification, therefore, a great benefit for public health.

Identification of new variants for prostate cancer has important diagnostic applications, as they can be used to identify those at particularly at risk for prostate cancer genetic susceptibility. Such variants can for example be incorporated in diagnostic applications that have already been developed. The present invention provides such variants.

### SUMMARY OF THE INVENTION

The present inventors have discovered that certain polymorphic markers are associated with risk of prostate cancer. Such markers are useful in a number of diagnostic applications, as described further herein. The markers can also be used in certain aspects that relate to development of markers for diagnostic use, systems and apparati for diagnostic use, as well as in methods that include selection of individuals based on their genetic status with respect to such variants. These and other aspects of the invention are described in more detail herein.

In one aspect the invention relates to a method of determining a susceptibility to prostate cancer, the method comprising obtaining nucleic acid sequence data about a human individual identifying at least one allele of at least one polymorphic marker, wherein different alleles of the at least one polymorphic marker are associated with different susceptibilities to prostate cancer in humans, and determining a susceptibility to prostate cancer from the nucleic acid sequence data, wherein the at least one polymorphic marker is selected from the group consisting of rs16902094, rs8102476, rs10934853 and rs445114, and markers in linkage disequilibrium therewith. In one embodiment, the nucleic acid sequence data is sequence data from a nucleic acid sample from the human individual.

In some variations, the methods of the invention further include a step, prior to the analyzing step, of obtaining the nucleic acid sequence data from a biological sample from the human individual, where the biological sample contains nucleic acid from the human individual. Many techniques are available for obtaining nucleic acid sequence data from a biological sample. In some variations, the obtaining of nucleic acid sequence data comprises a method that includes at least one procedure selected from amplifying nucleic acid from the biological sample, and performing a hybridization assay using a nucleic acid probe and nucleic acid from the biological sample (or using amplified nucleic acid obtain from amplifying nucleic acid from the biological sample).

In some variations of the methods disclosed herein, nucleic acid sequence data from the human individual is analyzed for at least one allele of at least two of said polymorphic markers, wherein different haplotypes comprising alleles of the at least two polymorphic markers are associated with different susceptibilities to prostate cancer in humans. In still other variations, nucleic acid sequence data from the individual is analyzed for at least two alleles of a polymorphic marker, or at least two alleles of two or more polymorphic markers. Analyses for all combinations of numbers of markers and alleles for the markers described herein are specifically contemplated, especially all combinations of two, three, or four of the markers rs16902094, rs8102476, rs10934853 and rs445114, or markers in linkage disequilibrium therewith. As described in further detail herein, polymorphic markers can comprise variations comprising one or more nucleotides at the nucleotide level. Sequence data indicative of a particular polymorphisms, in particular with respect to specific alleles of a polymorphism, is thus indicative of the nucleotides that are present at the specific polymorphic site(s) that characterize the polymorphism. For polymorphisms that comprise a single nucleotide, (so called single nucleotide polymorphisms (SNPs)), the sequence data thus includes at least sequence for the single nucleotide characteristic of the polymorphism.

In a related aspect, the invention includes a method of determining nucleic acid sequence data indicative of a susceptibility to prostate cancer, the method comprising: analyzing nucleic acid from a human individual to obtain nucleic acid data for at least one allele of at least one polymorphic marker selected from the group consisting of rs16902094, rs8102476, rs10934853 and rs445114, and markers in linkage disequilibirium therewith; wherein different alleles of the at least one polymorphic marker are associated with different susceptibilities to prostate cancer in humans, and preparing a report containing the nucleic acid sequence data for said at least one allele of the at least one polymorphic marker, wherein the report is written to a tangible medium such as a computer readable medium or printed on paper; or wherein the report is displayed on a visual display, such as a computer screen or other electronic display. Exemplary techniques for analyzing nucleic acid include any techniques that provide the sequence information of interest, including but not limited to techniques that include amplifying nucleic acid from a biological sample from the human individual; performing a hybridization assay using a nucleic acid probe and nucleic acid from the human individual, or from the results of such amplifying; or any available sequencing technologies (some of which involve amplification and hybridization steps).

The invention in another aspect relates to a method for determining a susceptibility to prostate cancer in a human individual, comprising determining the presence or absence of at least one allele of at least one polymorphic marker in a nucleic acid sample obtained from the individual, or in a genotype dataset from the individual, wherein the at least one polymorphic marker is selected from the group consisting of rs16902094, rs8102476, rs10934853 and rs445114, and markers in linkage disequilibrium therewith, and wherein determination of the presence of the at least one allele is indicative of a susceptibility to prostate cancer. In some variations, the susceptibility to prostate cancer is displayed on a visual display selected from the group consisting of an electronic display and a printed report. Further aspects of the methods comprise reporting the susceptibility to prostate cancer for the marker in linkage disequilibrium on a visual display, or recording the susceptibility in a computer-readable medium or printed report.

The invention also relates to a method of screening a candidate marker for assessing susceptibility to prostate cancer, comprising analyzing the frequency of at least one allele of at least one polymorphic marker selected from the group consisting of the markers set forth in Table 8, Table 9, Table 10 and Table 11, in a population of human individuals diagnosed with prostate cancer, wherein a significant difference in frequency of the at least one allele in the population of human individuals diagnosed with prostate cancer as compared to the frequency of the at least one allele in a control population of human individuals is indicative of the marker being useful as a susceptibility marker for prostate cancer.

Another aspect of the invention relates to a method of identification of a marker for use in assessing susceptibility to prostate cancer, the method comprising (a) identifying at least one polymorphic marker in linkage disequilibrium with at least one marker selected from the group consisting of rs16902094, rs8102476, rs10934853 and rs445114; (b) obtaining nucleic acid sequence data about a plurality of human individuals diagnosed with prostate cancer, and a plurality of control individuals, determining the presence or absence at least one allele of the at the least one polymorphic marker in the nucleic acid sequence data; and (c) determine the difference in frequency of the at least one allele between the individuals diagnosed with prostate cancer and the control group; wherein determination of a significant difference in frequency of the at least one allele is indicative of the at least one marker being useful for assessing susceptibility to prostate cancer.

The invention furthermore relates to a method of predicting prognosis of an individual diagnosed with prostate cancer, the method comprising obtaining nucleic acid sequence data about the human individual identifying at least one allele of at least one polymorphic marker selected from the group consisting of rs16902094, rs8102476, rs10934853 and rs445114, and markers in linkage disequilibrium therewith, wherein different alleles of the at least one polymorphic marker are associated with different susceptibilities to prostate cancer in humans, and predicting prognosis of the individual from the nucleic acid sequence data.

The invention in a further aspect relates to a method of assessing an individual for probability of response to a therapeutic agent for preventing, treating, and/or ameliorating symptoms associated with prostate cancer, comprising: determining the identity of at least one allele of at least one polymorphic marker in a nucleic acid sample obtained from the individual, or in a genotype dataset derived from the individual, wherein the at least one polymorphic marker is selected from the group consisting of rs16902094, rs8102476, rs10934853 and rs445114, and markers in linkage disequilibrium therewith, and wherein the identity of the at least one allele of the at least one marker is indicative of a probability of a positive response to the therapeutic agent.

With respect to any method of the invention that indicates an increased susceptibility to prostate cancer, a further variation of the invention further includes prescribing and/or administering to the human individual with the increased susceptibility a standard of care therapeutic for prostate health. Exemplary therapeutics include therapeutics for prostate cancer, used in a prophylactic context; therapeutics for benign prostate hypertrophy; and therapeutics believed to have a beneficial health effect or anticancer properties with respect to prostate.

The invention further relates to the use of an oligonucleotide probe in the manufacture of a diagnostic reagent for use in diagnosing and/or assessing susceptibility to prostate cancer in a human individual, wherein the probe hybridizes to a segment of a nucleic acid with sequence as set forth in any one of SEQ ID NO:1-978 that comprises at least one polymorphic site, and wherein the fragment is 15-400 nucleotides in length.

The invention also provides kits useful in the diagnostic applications described herein. Accordingly, in one aspect, the invention relates to a kit for assessing susceptibility to prostate cancer in a human individual, the kit comprising reagents for selectively detecting at least one allele of at least one polymorphic marker in the human genome of the human individual, wherein the polymorphic marker is selected from the group consisting rs16902094, rs8102476, rs10934853 and rs445114, and markers in linkage disequilibrium therewith, and a collection of data comprising correlation data between the at least one polymorphic marker and susceptibility to prostate cancer.

In various aspects, the kit contains reagents for selectively detecting at least one allele of at least two of said polymorphic markers. In further aspects, the reagents comprise, for each of said at least two polymorphic markers, at least one contiguous oligonucleotide that hybridizes to a fragment of the genome of the individual comprising the polymorphic marker. In still further aspects, the reagents comprise, for each polymorphic marker, at least two contiguous oligonucleotides that hybridize to a fragment of the human genome comprising the polymorphic marker, wherein each of the at least two oligonucleotides selectively recognize a different allele of the polymorphic marker. The present disclosure also contemplates, in various aspects, that at least one of the oligonucleotides contains a detectable label.

A collection of data is not an essential element to all kits of the invention. In some variations, the invention includes a kit for assessing susceptibility to prostate cancer in a human individual, the kit comprising reagents for selectively detecting at least one allele of at least two polymorphic marker in the human genome, wherein the at least two polymorphic markers are selected from the group consisting of rs16902094, rs8102476, rs10934853 and rs445114, and markers in linkage disequilibrium therewith.

Computer-implemented aspects of the invention include computer-readable media and computer systems and apparati. One aspect relates to a computer-readable medium having computer executable instructions for determining susceptibility to prostate cancer, the computer readable medium comprising: data identifying at least one allele of at least one polymorphic marker for at least one human subject; a routine stored on the computer readable medium and adapted to be executed by a processor to determine risk of developing prostate cancer for the at least one polymorphic marker for the subject; wherein the at least one polymorphic marker is selected from the group consisting of rs16902094, rs8102476, rs10934853 and rs445114, and markers in linkage disequilibrium therewith.

Another computer-implemented aspect relates to an apparatus for determining a genetic indicator for prostate cancer in a human individual, comprising a processor, and a computer readable memory having computer executable instructions adapted to be executed on the processor to analyze marker and/or haplotype information for at least one human individual with respect to at least one polymorphic marker selected from the group consisting of rs16902094, rs8102476, rs10934853 and rs445114, and markers in linkage disequilibrium therewith, and generate an output based on the marker or haplotype information, wherein the output comprises a measure of susceptibility of the at least one marker or haplotype as a genetic indicator of prostate cancer for the human individual.

These and other aspects of the invention will be described in detail in the following, and all such features are intended as aspects of the invention. Particular embodiments will be described, in particular as they relate to the selection and use of polymorphic variants and haplotypes. It should be understood that all combinations of features described herein in the following are contemplated, even if the combination of feature is not specifically found in the same sentence or paragraph herein. This includes in particular the use of all markers disclosed herein, alone or in combination, for analysis individually or in haplotypes, in all aspects of the invention as described herein.

Aspects of the invention described with the term “comprising” should be understood to include the elements explicitly listed, and optionally, additional elements. Aspects of the invention described with “a” or “an” should be understood to include “one or more” unless the context clearly requires a narrower meaning.

Moreover, features of the invention described herein can be re-combined into additional embodiments that also are intended as aspects of the invention, irrespective of whether the combination of features is specifically mentioned above as an aspect or embodiment of the invention. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention, even if, for brevity, every individual member has not been specifically mentioned herein. When aspects of the invention that are described herein as being selected from a genus, it should be understood that the selection can include mixtures of two or more members of the genus. Similarly, with respect to aspects of the invention that have been described as a range, such as a range of values, every sub-range within the range is considered an aspect of the invention.

In addition to the foregoing, the invention includes, as an additional aspect, all embodiments of the invention narrower in scope in any way than the variations specifically described herein. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. In all cases, claims should be construed to cover only subject matter eligible for protection under the patent statute.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 shows a schematic view of the 8q24 region. Shown are, from top to bottom, the currently described and previously reported three prostate- and one breast cancer risk variants on 8q24, the pairwise correlation (r²) between SNPs based on the CEU HapMap data, and the HapMap recombination hotspots and recombination rates.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “3 rs16902094” refers to the 3 allele of marker rs16902094 being in the haplotype, and is equivalent to “rs16902094 allele 3” and “rs16902094-3”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of prostate cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of prostate cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “prostate cancer therapeutic agent” refers to an agent that can be used to ameliorate or prevent symptoms associated with prostate cancer.

The term “prostate cancer-associated nucleic acid”, as described herein, refers to a nucleic acid that has been found to be associated to prostate cancer. This includes, but is not limited to, the markers and haplotypes described herein and markers and haplotypes in strong linkage disequilibrium (LD) therewith. In one embodiment, a prostate cancer-associated nucleic acid refers to an LD-block found to be associated with Type 2 diabetes through at least one polymorphic marker located within the LD block.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to a corresponding contiguous bases in a target nucleic acid sequence. The backbone is composed of subunit backbone moieties supporting the purine an pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “LD Block C19”, as described herein, refers to the Linkage Disequilibrium (LD) block on Chromosome 19 between markers rs8110367 and rs2304150, corresponding to positions 43,170,305-43,647,423 of NCBI (National Center for Biotechnology Information) Build 36. “LD Block C03”, as described herein, refers to the Linkage Disequilibrium (LD) block on Chromosome 3 between markers rs497-4416 and rs2659698, corresponding to positions 129,060,479-129,709,054 of NCBI (National Center for Biotechnology Information) Build 36. The term “LD Block C08A”, as described herein, refers to the Linkage Disequilibrium (LD) block on Chromosome 8 between markers rs1840709 and rs731900, corresponding to positions 128,168,637-128,459,842 of NCBI (National Center for Biotechnology Information) Build 36. The term “LD Block C08B”, as described herein, refers to the Linkage Disequilibrium (LD) block on Chromosome 8 between markers rs13280181 and rs7015780, corresponding to positions 128,355,698-128,458,689 of NCBI (National Center for Biotechnology Information) Build 36.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome. For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including mini- and microsatellites, and insertions, deletions and inversions (also called copy number variations (CNVs)). A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

Due to their abundance, SNPs account for a majority of sequence variation in the human genome. Over 6 million SNPs have been validated to date (http://www.ncbi.nlm.nih.gov/projects/SNP/snp_summary.cgi). However, CNVs are receiving increased attention. These large-scale polymorphisms (typically 1 kb or larger) account for polymorphic variation affecting a substantial proportion of the assembled human genome; known CNVs covery over 15% of the human genome sequence (Estivill, X Armengol; L., PloS Genetics 3:1787-99 (2007). A http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the markers described herein to be associated with prostate cancer. Methods for detecting CNVs include comparative genomic hybridization (CGH) and genotyping, including use of genotyping arrays, as described by Carter (Nature Genetics 39:S16-S21 (2007)). The Database of Genomic Variants (http://projects.tcag.ca/variation/) contains updated information about the location, type and size of described CNVs. The database currently contains data for over 15,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the complimentary strand on the DNA template, the presence of the complementary bases T and C can be measured. Quantitatively (for example, in terms of risk estimates), identical results would be obtained from measurement of either DNA strand (+ strand or − strand).

Polymorphic markers (variants) can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurence of each allele or haplotype in the population. For populations of diploids, e.g., human populations, individuals will typically have two alleles or allelic combinations for each genetic element (e.g., a marker, haplotype or gene).

Many different measures have been proposed for assessing the strength of linkage disequilibrium (LD; reviewed in Devlin, B. & Risch, N., Genomics 29:311-22 (1995))). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, or at least 0.99. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium is defined as r²>0.2 and |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian, african, japanese, chinese), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with the variants described herein may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (such as <10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a susceptibility region, for example within an LD block, association of all possible combinations of genotyped markers within the region is studied. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

### Statistical Analysis

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families to the study, first and second-degree relatives can be eliminated from the patient list. Furthermore, the test can be repeated for association correcting for any remaining relatedness among the patients, by extending a variance adjustment procedure previously described (Risch, N. & Teng, J. Genome Res., 8:1273-1288 (1998)) for sibships so that it can be applied to general familial relationships, and present both adjusted and unadjusted p-values for comparison. The method of genomic controls (Devlin, B. & Roeder, K. Biometrics 55:997 (1999)) can also be used to adjust for the relatedness of the individuals and possible stratification. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, ideally from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold are a measure of a true genetic effect, and replication in additional cohorts is not necessarily from a statistical point of view. Importantly, however, signals with P-values that are greater than this threshold may also be due to a true genetic effect. Thus, since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Methods of Determining Susceptibility to Prostate Cancer

It has been shown for the first time that certain polymorphic variants on chromosome 3q21.3, chromosome 8q24.21 and chromosome 19q13.2 are associated with risk of developing prostate cancer. Certain alleles of certain polymorphic markers have been found to be present at increased frequency in individuals with diagnosis of prostate cancer compared with controls. These polymorphic markers are thus associated with risk of prostate cancer. Without intending to being bound to a particular theory, the particular polymorphic markers described herein, as well as markers in linkage disequilibrium with these polymorphic markers, are contemplated to be useful as markers for determining susceptibility to prostate cancer. These markers are believed to be useful in a range of diagnostic applications, as described further herein.

Association on 3q21.3 is in a region that contains several genes. For example, SNP rs10934853 is located in the fourth intron of the EEFSEC gene, which is an elongation factor required for effective selenoprotein translation. Other RefSeq genes in the same LD region (LD Block C03) are SEC61A1 and RUVBL1. None of these genes has previously been directly implicated in prostate cancer. On 19q13.2, association is found in a LD-region (LD Block C19) with several annotated RefSeq genes. One of these is PPP1R14A, a gene reported to be an inhibitor of smooth muscle myosin phosphatase.

Based on a genome-wide SNP association study and a follow up study on prostate cancer, four variants, and their correlated surrogate variants, were shown to be associated with the disease in European populations; rs10934853 (SEQ ID NO: 1) on 3q21.3, rs16902094 (SEQ ID NO: 2), rs16902104 (SEQ ID NO:287) and rs445114 (SEQ ID NO: 3) on 8q24.21 and rs8102476 (SEQ ID NO: 4) on 19q13.2. For these markers, the risk alleles rs10934853-A, rs16902094-G, rs16902104-T and rs445114-T on 8q24.21 and rs8102476-C on 19q13.2 were found, with OR values ranging from 1.12 to 1.21 and all with P-values of association with prostate cancer less than 5×10⁻¹⁰. Exemplary surrogate variants (surrogate markers) of these variants are shown in Tables 8-11 and 17-20 herein.

Accordingly, in one aspect the invention provides a method of determining a susceptibility to prostate cancer, the method comprising obtaining nucleic acid sequence data about a human individual identifying at least one allele of at least one polymorphic marker, wherein different alleles of the at least one polymorphic marker are associated with different susceptibilities to prostate cancer in humans, and determining a susceptibility to prostate cancer from the nucleic acid sequence data, wherein the at least one polymorphic marker is selected from the group consisting of rs16902094, rs8102476, rs10934853 and rs445114, and markers in linkage disequilibirium therewith. Nucleic acid sequence data identifying particular alleles of polymorphic markers is sometimes also referred to as genotype data. In one embodiment, nucleic acid sequence data is obtained from a biological sample from the individual.

Nucleic acid sequence data can be obtained for example by analyzing sequence of the at least one polymorphic marker in a biological sample from the individual. Alternatively, nucleic acid sequence data can be obtained in a genotype dataset from the human individual and analyzing sequence of the at least one polymorphic marker in the dataset. Such analysis in certain embodiments comprises determining the presence or absence of a particular allele of specific polymorphic markers.

In certain embodiments, the method comprises steps of (i) obtaining a nucleic acid sample from an individual; (ii) determine the nucleic acid sequence of at least one polymorphic marker in the nucleic acid sample; and (iii) determine a susceptibility to prostate cancer from the nucleic acid sequence of the at least one polymorphic marker.

In certain embodiments, the markers in linkage disequilibrium with rs8102476 are selected from the group consisting of rs8102476, rs8110367, rs10500278, rs705503, rs1654338, rs4803899, rs1036233, rs7246060, rs8102476, rs12976534, rs4803934, rs11668070, rs7250689, rs7253245, rs3786870, rs3786872, rs3786877, rs12610791, rs8101725, rs870218, rs12611009, rs3826896, rs8104823, rs1821284, rs4802327, rs11672219, rs3816044, rs2304177, rs4312417, rs3178327, rs3900981, rs3843754, rs2302182, rs1052375, rs12609246, rs3745843, rs3745844, and rs2304150, which are the markers listed in Table 11. In certain embodiments, markers in linkage disequilibrium with rs10934853 are selected from the group consisting of rs10934853, rs4974416, rs13095214, rs11923862, rs1543272, rs6439086, rs7644239, rs7625264, rs11921463, rs13080277, rs11926127, rs7649674, rs7616277, rs6439094, rs16838982, rs2053016, rs17203687, rs16845806, rs7630727, rs1549876, rs17282209, rs6439104, rs1469659, rs7611430, rs6770337, rs6777095, rs4602341, rs4857833, rs6439108, rs6764517, rs981447, rs981446, rs1469658, rs2335772, rs1030656, rs1030655, rs2335771, rs759945, rs2075402, rs1554534, rs3732402, rs13091198, rs11714052, rs6439113, rs6787614, rs11720239, rs11715661, rs7641133, rs11924142, rs7650365, rs6788879, rs6439115, rs4857836, rs4857837, rs11707462, rs9821568, rs6784159, rs2811475, rs13095660, rs6439116, rs6414310, rs2955102, rs11920225, rs11709066, rs11716941, rs2811472, rs13077913, rs13077790, rs2811473, rs2687728, rs10934850, rs872267, rs2687731, rs3122174, rs2999051, rs13067650, rs2248668, rs2955121, rs11706455, rs2999052, rs11715394, rs2687729, rs2811478, rs2999060, rs2999056, rs2955123, rs2811517, rs2811516, rs2811515, rs2811514, rs2811512, rs2811511, rs883238, rs940061, rs2811510, rs2811483, rs2811484, rs2687730, rs2811509, rs2492285, rs2687720, rs2811508, rs2811486, rs6439119, rs2955125, rs2955126, rs2955127, rs4293718, rs2955129, rs7374072, rs2999090, rs7372439, rs4857871, rs4857872, rs4857873, rs6770140, rs4384971, rs2999089, rs6439121, rs2254379, rs2955130, rs9814834, rs2955132, rs9845651, rs6439122, rs9873786, rs4857838, rs6775988, rs9830294, rs4857877, rs2999086, rs2999085, rs2999084, rs2999083, rs2999081, rs2999079, rs4074440, rs2955077, rs9843281, rs2999073, rs2955085, rs2999072, rs13434079, rs2955088, rs2999070, rs17343355, rs2955090, rs2955091, rs2999069, rs2955092, rs2955094, rs2955095, rs2955096, rs2999068, rs2999067, rs2955099, rs2999066, rs2999065, rs2811545, rs2999035, rs2811544, rs2811543, rs2811541, rs2811540, rs2811539, rs2811538, rs2811396, rs2811400, rs2811537, rs2999064, rs2811536, rs2811534, rs2811413, rs2811415, rs2811533, rs2811416, rs2811532, rs2811531, rs2955100, rs2999061, rs2811529, rs2811527, rs2811373, rs2811525, rs7374952, rs7374227, rs4593050, rs6439124, rs7373998, rs2955101, rs2811519, rs2811518, rs2955103, rs2811388, rs2999036, rs2811390, rs2811391, rs2811393, rs2037965, rs2811397, rs6805582, rs6805621, rs6794591, rs16843876, rs11706852, rs11706826, rs11706908, rs6771646, rs13095166, rs10934853, rs12486127, rs12486156, rs11708733, rs6772407, rs4857841, rs11710704, rs16844002, rs6798749, rs1735558, rs4857879, rs11721213, rs1735549, rs1735546, rs12632366, rs1735545, rs1702122, rs1108313, rs1735538, rs1702119, rs1702118, rs3021461, rs2977565, rs2293947, rs741925, rs729847, rs1702134, rs1620440, rs7632169, rs1735527, rs760383, rs11705709, rs11705891, rs2999031, rs6780368, rs2659685, rs11715947, rs1735537, rs11717030, rs2977564, rs2939820, rs3828417, rs4527399, rs4521245, rs1806462, rs2860228, rs9851497, rs6789646, rs7629791, rs2713576, and rs2659698, which are the markers listed in Table 8. In certain embodiments, markers in linkage disequilibrium with rs16902094 are selected from the group consisting of rs16902094, rs1840709, rs3857883, rs1456316, rs1456315, rs7006409, rs4871775, rs4871779, rs13251915, rs283720, rs283704, rs283705, SG08S1723, rs453875, SG08S1738, rs11785664, rs622556, rs452529, rs400818, rs386883, rs377649, rs432470, rs424281, rs16902103, rs16902104, rs1668875, rs7002712, rs587948, rs623401, rs16902118, rs10095860, rs16902121, rs13256275, rs11785277, rs11774827, rs11782693, rs11782700, rs11782735, rs11783559, rs11783615, rs11784125, rs11776260, rs11774907, rs16902127, rs7015780, and rs731900, which are the markers listed in Table 9. In certain embodiments, markers in linkage disequilibrium with rs445114 are selected from the group consisting of rs13280181, rs12707923, rs6984900, rs17450865, rs7822551, rs12549518, rs6996866, rs2007197, rs283727, rs283728, rs283704, rs283705, rs10107982, rs453875, rs445114, rs11785664, rs622556, rs452529, rs13256367, rs10956356, rs10956358, rs7008928, rs7009077, rs400818, rs386883, rs377649, rs432470, rs424281, rs1668875, rs7002712, rs587948, rs623401, rs10956359, rs17464492, rs420101, rs7838714, rs389143, rs688201, rs687324, rs687279, rs436238, rs581761, rs673745, rs688937, rs672888, rs7826557, rs418269, rs385278, rs391640, rs670725, rs382824, rs383205, rs373616, rs13275275, rs13248140, rs10956361, rs10956362, rs13249993, rs11777532, rs10956363, rs4871782, rs10087810, rs12541832, rs13262406, rs10098985, rs13281615, rs13256275, rs13267780, rs10447995, rs7014657, rs7002826, rs7007568, rs7842494, rs5022926, rs9693995, rs2121629, rs978683, rs9283954, rs7831303, rs7815100, rs4143118, rs6988647, rs9693143, rs2060775, rs10956364, rs11776330, rs7845452, rs7815245, rs2121631, rs1562430, rs2392780, rs7015780, which are the markers listed in Table 10.

Further surrogate markers are provided in Tables 17-20 herein. Thus, in certain embodiments, markers in linkage disequilibrium with rs8102476 may also be selected from the group consisting of the markers listed in Table 20. Likewise, in certain embodiments, markers in linkage disequilibrium with rs10934853 may also be selected from the group consisting of the markers listed in Table 17; markers in linkage disequilibrium with rs16902094 may also be selected from the group consisting of the markers listed in Table 18; and markers in linkage disequilibrium with rs445114 are selected from the group consisting of the markers listed in Table 19.

Surrogate markers can be selected based on certain values of the linkage disequilibrium measures D′ and r², as described further herein. Markers that are in linkage disequilibrium with the markers rs16902094, rs10934853, rs445114 and rs8102476 are exemplified by the markers listed in Tables 8-11 and 17-20 herein, but the skilled person will appreciate that other markers in linkage disequilibrium with these markers may also be used in the diagnostic applications described herein. Further, the skilled person will appreciate that since linkage disequilibrium is a continuous measure, certain values of the LD measures D′ and r² may be suitably chosen to define markers that are useful as surrogate markers in LD with the markers described herein. The values of D′ and r² given in Tables 8-11 and 17-20 may in certain embodiments be used to define such marker subsets of the markers listed in the Tables 8-11 and Tables 17-20. In one such embodiment, suitable markers in linkage disequilibrium are correlated with the anchor marker by values of r² greater than 0.2. In another such embodiment, suitable markers in linkage disequilibrium are correlated with the anchor marker by values of r² greater than 0.5. In yet another such embodiment, suitable markers in linkage disequilibrium are correlated with the anchor marker by values of r² greater than 0.8. In one embodiment, suitable markers in linkage disequilibrium are correlated with the anchor marker by values of r² of 1.0. Such markers are perfect surrogates of the anchor marker, and will give identical association results, i.e. they provide identical genetic information.

Association data presented in Tables 13-16 (Example 2) show exemplary results of association of surrogate markers in an Iceland sample set. Surrogate markers give different association signals because they are in different linkage disequilibrium with the underlying signal. For example, for marker rs445114, the markers rs453875, rs13280181 and rs581761 give different association results. The strongest signal is observed for rs453875 (OR 1.20, P-value 6.1E-7), while weaker association is observed for rs13280181 (OR 1.15, P-value 0.002) and rs581761 (OR 1.05, P-value 0.14). All three are surrogates for rs445114, but capture the underlying association signal to a varying degree. It should also be noted that sample size has an effect of the power to detect an underlying association. Therefore, association values for a sample size of 1776 cases and 35675 controls, as shown in Table 14, are weaker than would have been obtained using the extended sample sets as shown in Table 1. This does not mean that the inherent value of each surrogate marker is affected, but is rather a manifestation of the relative strength of such markers in capturing the underlying association.

Accordingly, in certain embodiments, surrogate markers of rs10934853 are selected from the group consisting of the markers listed in Table 13. In certain embodiments, surrogate markers of rs445114 are selected from the group consisting of the markers listed in Table 14. In certain embodiments, surrogate markers of rs16902094 are selected from the group consisting of the markers listed in Table 15. In certain embodiments, surrogate markers of rs8102476 are selected from the group consisting of the markers listed in Table 16.

In one embodiment, surrogate markers of rs10934853 are selected from the group consisting of rs16845806, rs7630727, rs1549876, rs6439104, rs1469659, rs7611430, rs6770337, rs6777095, rs4602341, rs4857833, rs6439108, rs6764517, rs981447, rs981446, rs1469658, rs2335772, rs1030656, rs1030655, rs2335771, rs759945, rs2075402, rs1554534, rs3732402, rs6439113, rs7641133, rs11924142, rs7650365, rs6788879, rs6439115, rs4857836, rs4857837, rs9821568, rs2811475, rs6414310, rs2955102, rs11920225, rs2811472, rs2811473, rs2687728, rs872267, rs2687731, rs3122174, rs2999051, rs2248668, rs2955121, rs2999052, rs2687729, rs2999060, rs2999056, rs2955123, rs2811517, rs2811516, rs2811515, rs2811514, rs2811512, rs883238, rs940061, rs2811510, rs2811483, rs2811484, rs2811509, rs2492285, rs2687720, rs2811508, rs6439119, rs2955125, rs2955127, rs7374072, rs7372439, rs4857871, rs4857872, rs4857873, rs6770140, rs4384971, rs6439121, rs2254379, rs9814834, rs2955132, rs9845651, rs6439122, rs9873786, rs4857838, rs6775988, rs9830294, rs4857877, rs4074440, rs9843281, rs13434079, rs17343355, rs2999035, rs2999064, rs2811413, rs2811529, rs2955103, rs13095166, rs12486127, rs12486156, rs4857841, rs1735558, rs4857879, rs1735549, rs1735546, rs1735545, rs1702122, rs1735538, rs1702119, rs1702118, rs3021461, rs2977565, rs741925, rs729847, rs1702134, rs1620440, rs7632169, rs1735527, rs760383, rs6780368, rs2659685, rs1735537, and rs2977564.

In one embodiment, surrogate markers of rs445114 are selected from the group consisting of rs453875, rs10107982, rs13256367, rs1668875, rs587948, rs623401, rs10956359, rs17464492, rs7822551, rs17450865, rs2007197, rs6984900, rs12707923, rs13280181, rs13262081, rs620861, rs391640, and rs13267780.

In one embodiment, surrogate markers of rs16902094 are selected from the group consisting of rs16902103, rs13251915, rs453875, rs283720, rs1668875, rs587948, and rs623401.

In one embodiment, surrogate markers of rs445114 are selected from the group consisting of rs4803899, rs1036233, rs7246060, rs12976534, rs4803934, rs11668070, and rs7250689.

In preferred embodiments, the markers useful in the methods of the invention are selected from the group consisting of rs16902094, rs10934853, rs445114, rs8102476, rs620861 and rs16902104. In one preferred embodiment, the marker is rs8102476. In another preferred embodiment, the marker is rs10934853. In another preferred embodiment, the marker is rs16902094. In another preferred embodiment, the marker is rs445114. In another embodiment, the marker is rs620861. In another embodiment, the marker is rs16902104.

In certain embodiments of the invention, sequence data obtained about a polymorphic marker is amino acid sequence data. Polymorphic markers can result in alterations in the amino acid sequence of encoded polypeptide or protein sequence. In certain embodiments, the analysis of amino acid sequence data comprises determining the presence or absence of an amino acid substitution in the amino acid encoded by the at least one polymorphic marker. Sequence data can in certain embodiments be obtained by analyzing the amino acid sequence encoded by the at least one polymorphic marker in a biological sample obtained from the individual.

To define markers that are useful in diagnostic for determining a susceptibility to prostate cancer, it may be useful to compare the frequency of markers alleles in individuals with prostate cancer to their corresponding frequency in control individuals. In one embodiment, an increase in frequency of the at least one allele in the at least one polymorphism in individuals diagnosed with prostate cancer, as compared with the frequency of the at least one allele in the control group is indicative of the at least one allele being useful for assessing increased susceptibility to prostate cancer.

In another embodiment, a decrease in frequency of the at least one allele in the at least one polymorphism in individuals diagnosed with prostate cancer, as compared with the frequency of the at least one allele in the control sample is indicative of the at least one allele being useful for assessing decreased susceptibility to, or protection against, prostate cancer.

In general, sequence data can be obtained by analyzing a sample from an individual, or by analyzing information about specific markers in a genotype database. In certain embodiments, sequence data can be obtained through nucleic acid sequence information or amino acid sequence information from a preexisting record about a human individual. Such a preexisting record can be any documentation, database or other form of data storage containing such information.

Determination of a susceptibility or risk of a particular individual in general comprises comparison of the genotype information (sequence information about particular marker or a plurality of markers) to a record or database providing a correlation about particular polymorphic marker(s) and susceptibility to prostate cancer. Thus, in specific embodiments, determining a susceptibility comprises comparing the sequence data to a database containing correlation data between the at least one polymorphic marker and susceptibility to prostate cancer. In certain embodiments, the database comprises at least one measure of susceptibility to prostate cancer for the at least one polymorphic marker. In certain embodiments, the database comprises a look-up table comprising at least one measure of susceptibility to prostate cancer for the at least one polymorphic marker. Determination of susceptibility is based on sequence information about particular markers identifying particular alleles at those markers. A calculation of susceptibility (risk) of prostate cancer is performed based on the information, using risk measures that have been determined for the particular alleles or combination of alleles. The measure of susceptibility may in the form of relative risk (RR), absolute risk (AR), percentage (%) or other convenient measure for describing genetic susceptibility of individuals.

Certain embodiments of the invention relate to markers located within the LD Block C19, LD Block C03, LD Block C08A and/or LD Block C08B as defined herein. These LD Blocks contain markers that are associated with risk of prostate cancer, as shown herein. For example, LD Block C19 comprises markers in linkage disequilibrium with rs8102476, LD Block C03 comprises markers in linkage disequilibrium with rs10934853, LD Block C08A comprises markers in linkage disequilibrium with rs16902094 and LD Block C08B comprises markers in linkage disequilibrium with rs445114. It is however also contemplated that surrogate markers useful for determining susceptibility to prostate cancer may be located outside these blocks as defined in physical terms (genomic locations). Thus, other embodiments of the invention are not confined to markers located within the physical boundaries of the LD blocks as defined. Rather such embodiments relate to useful surrogate markers due to being in LD with one or more of the markers shown herein to be associated with risk of prostate cancer.

Another aspect of the invention relates to a method for determining a susceptibility to prostate cancer in a human individual, comprising determining the presence or absence of at least one allele of at least one polymorphic marker in a nucleic acid sample obtained from the individual, or in a genotype dataset from the individual, wherein the at least one polymorphic marker is selected from the group consisting of rs16902094, rs10934853, rs445114 and rs8102476, and markers in linkage disequilibrium therewith, and wherein determination of the presence of the at least one allele is indicative of a susceptibility to prostate cancer. Determination of the presence of an allele that correlates with prostate cancer is indicative of an increased susceptibility (increased risk) to prostate cancer. Individuals who are homozygous for such alleles are particularly susceptible to prostate cancer. On the other hand, individuals who do not carry such at-risk alleles are at a decreased susceptibility of developing prostate cancer. For SNPs, such individuals will be homozygous for the alternate (protective) allele of the polymorphism.

Determination of susceptibility is in some embodiments reported using non-carriers of the at-risk alleles of polymorphic markers as a reference. In certain embodiments, susceptibility is reported based on a comparison with the general population, e.g. compared with a random selection of individuals from the population. Such embodiments thus reflect the susceptibility (risk) of an individual compared with a randomly selected individual from the population.

In certain embodiments, polymorphic markers are detected by sequencing technologies. Obtaining sequence information about an individual identifies particular nucleotides in the context of a sequence. For SNPs, sequence information about a single unique sequence site is sufficient to identify alleles at that particular SNP. For markers comprising more than one nucleotide, sequence information about the genomic region of the individual that contains the polymorphic site identifies the alleles of the individual for the particular site. The sequence information can be obtained from a sample from the individual. In certain embodiments, the sample is a nucleic acid sample. In certain other embodiments, the sample is a protein sample.

Various methods for obtaining nucleic acid sequence are known to the skilled person, and all such methods are useful for practicing the invention. Sanger sequencing is a well-known method for generating nucleic acid sequence information. Recent methods for obtaining large amounts of sequence data have been developed, and such methods are also contemplated to be useful for obtaining sequence information. These include pyrosequencing technology (Ronaghi, M. et al. Anal Biochem 267:65-71 (1999); Ronaghi, et al. Biotechniques 25:876-878 (1998)), e.g. 454 pyrosequencing (Nyren, P., et al. Anal Biochem 208:171-175 (1993)), Illumina/Solexa sequencing technology (http://www.illumina.com; see also Strausberg, R L, et al Drug Disc Today 13:569-577 (2008)), and Supported Oligonucleotide Ligation and Detection Platform (SOLID) technology (Applied Biosystems, http://www.appliedbiosystems.com); Strausberg, R L, et al Drug Disc Today 13:569-577 (2008).

It is possible to impute or predict genotypes for un-genotyped relatives of genotyped individuals. For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypes of relatives of case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin, B. et al., Nat Genet 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I=I_{g}+Iᵤ, and denoting the number of genotyped cases with N, the effective sample size due to the un-genotyped cases is estimated by

$$\frac{{I}_{u}}{{I}_{g}} N .$$

It is also possible to impute genotypes for markers with no genotype data. For example, using the IMPUTE software (Marchini, J. et al. Nat Genet 39:906-13 (2007)) and the HapMap CEU data (for example NCBI Build 36 (db126b)) as reference (Frazer, K. A., et al. Nature 449:851-61 (2007)) it is possible to impute ungenotyped markers. This can be useful for extending genotype coverage, if the CEU dataset has been genotyped.

In the present context, and individual who is at an increased susceptibility (i.e., increased risk) for prostate cancer, is an individual in whom at least one specific allele at one or more polymorphic marker or haplotype conferring increased susceptibility (increased risk) for prostate cancer is identified (i.e., at-risk marker alleles or haplotypes). The at-risk marker or haplotype is one that confers an increased risk (increased susceptibility) of prostate cancer. In one embodiment, significance associated with a marker or haplotype is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.05, including but not limited to: at least 1.10, at least 1.11, at least 1.12, at least 1.13, at least 1.14, at least 1.15, at least 1.16, at least 1.17, at least 1.18, at least 1.19, at least 1.20, at least 1.30, at least 1.40, at least 1.50, at least 1.60, at least 1.70, at least 1.80, at least 1.90, and at least 2.0. In a particular embodiment, a risk (relative risk and/or odds ratio) of at least 1.08 is significant. In another particular embodiment, a risk of at least 1.13 is significant. In yet another embodiment, a risk of at least 1.19 is significant. Other cutoffs are also contemplated, e.g., at least 1.15, 1.25, 1.35, and so on, and such cutoffs are also within scope of the present invention. In other embodiments, a significant increase in risk is at least 15%, 16%, 17%, 18%, 19%, 20%, 25%, 30%, 35%, 40%, 45%, 50%, 55%, 60%, 65%, 70%, 75%, 80%, 85%, 90%, 95%, and at least 100%. In one particular embodiment, a significant increase in risk is at least 8%. In another particular embodiment, a significant increase in risk is at least 13%. In another particular embodiment, a significant increase in risk is at least 19%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype as described herein is one where at least one allele of at least one marker or haplotype is more frequently present in an individual at risk for prostate cancer (affected), or diagnosed with prostate cancer, compared to the frequency of its presence in a comparison group (control), such that the presence of the marker or haplotype is indicative of susceptibility to prostate cancer. The control group may in one embodiment be a population sample, i.e. a random sample from the general population. In another embodiment, the control group is represented by a group of individuals who are disease-free. Such disease-free controls may in one embodiment be characterized by the absence of one or more specific disease-associated symptoms. Alternatively, the disease-free controls are those that have not been diagnosed with prostate cancer. In another embodiment, the disease-free control group is characterized by the absence of one or more disease-specific risk factors. Such risk factors are in one embodiment at least one environmental risk factor. Representative environmental factors are risk factors related to lifestyle, including but not limited to food and drink habits, geographical location of main habitat, and occupational risk factors. In another embodiment, the risk factors comprise at least one additional genetic risk factor for prostate cancer.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

In other embodiments of the invention, an individual who is at a decreased susceptibility (i.e., at a decreased risk) for a disease (e.g., prostate cancer) is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk (or odds ratio) of less than 0.95, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.90. In another embodiment, significant decreased risk is less than 0.85. In yet another embodiment, significant decreased risk is less than 0.80. In another embodiment, the decrease in risk (or susceptibility) is at least 8%, including but not limited to at least 13%, at least 19%, at least 20%, at least 25%, at least 30%, at least 35%, at least 40%, at least 45%, and at least 50%. In one particular embodiment, a significant decrease in risk is at least about 8%. In another embodiment, a significant decrease in risk is at least about 13%. In another embodiment, the decrease in risk is at least about 19%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNPs), and wherein one allele is found in increased frequency in a group of individuals with prostate cancer, compared with controls, the other allele of the marker will be found in decreased frequency in the group of individuals with prostate cancer, compared with controls. In such a case, one allele of the marker (the one found in increased frequency in individuals with prostate cancer) will be the at-risk allele, while the other allele will be a protective allele.

A genetic variant associated with a disease or a trait can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2p; where n is the number autosomal loci and p the number of gonosomal (sex chromosomal) loci. Overall risk assessment calculations for a plurality of risk variants usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (rs2710646, rs16901979, rs1447295, rs6983267, rs7947353, rs1859962, rs4430796 and rs5945572; Gudmundsson, J. et al. Nat Genet 40:281-3 (2008); Gudmundsson, J., et al., Nat Genet 39:631-7 (2007), Gudmundsson, J., et al., Nat Genet 39:977-83 (2007); Yeager, M., et al, Nat Genet 39:645-49 (2007), Amundadottir, L., et al., Nat Genet 38:652-8 (2006); Thomas, G. et al. Nat Genet 40:310-15 (2008); Eeles, R. A., et al. Nat Genet 40:316-21 (2008)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Combining the additional risk factors for prostate cancer described herein, can be performed in an analogous fashion. Any one, or a combination of, the markers conferring increased risk of prostate cancer described herein, can be evaluated to perform overall risk assessment of prostate cancer. The variants can also be combined with any other genetic markers conferring risk of prostate cancer.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

The creation of a model to calculate the overall genetic risk involves two steps: i) conversion of odds-ratios for a single genetic variant into relative risk and ii) combination of risk from multiple variants in different genetic loci into a single relative risk value.

Deriving risk from odds-ratios. Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds-ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds-ratio.

It is well known that under the rare disease assumption, the relative risk of a disease can be approximated by the odds-ratio. This assumption may however not hold for many common diseases. Still, it turns out that the risk of one genotype variant relative to another can be estimated from the odds-ratio expressed above. The calculation is particularly simple under the assumption of random population controls where the controls are random samples from the same population as the cases, including affected people rather than being strictly unaffected individuals. To increase sample size and power, many of the large genome-wide association and replication studies used controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds-ratio of individuals is the same as the risk-ratio between these variants:

OR=Pr(A|c)/Pr(A|nc)=r

And likewise for the multiplicative model, where the risk is the product of the risk associated with the two allele copies, the allelic odds-ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

The risk relative to the average population risk. It is most convenient to represent the risk of a genetic variant relative to the average population since it makes it easier to communicate the lifetime risk for developing the disease compared with the baseline population risk. For example, in the multiplicative model we can calculate the relative population risk for variant “aa” as:

RR(aa)=Pr(A|aa)/Pr(A)=(Pr(A|aa)/Pr(A|bb))/(Pr(A)/Pr(A|bb))=r²/(Pr(aa)r²+Pr(ab)r+Pr(bb))=r²/(p²r²+2pqr+q²)=r²/R

Here “p” and “q” are the allele frequencies of “a” and “b” respectively. Likewise, we get that RR(ab)=r/R and RR(bb)=1/R. The allele frequency estimates may be obtained from the publications that report the odds-ratios and from the HapMap database. Note that in the case where we do not know the genotypes of an individual, the relative genetic risk for that test or marker is simply equal to one.

As an example, for prostate cancer risk, allele C of the disease associated marker rs8102476 on chromosome 19 has an allelic OR of 1.13 and a frequency (p) around 0.51 in white populations (Table 1). The genotype relative risk compared to genotype TT (homozygous for the alternate allele of rs8102476) are estimated based on the multiplicative model.

For CC it is 1.13×1.13=1.28; for CT it is simply the OR 1.13, and for TT it is 1.0 by definition.

The frequency of allele T is q=1−p=1−0.51=0.49. Population frequency of each of the three possible genotypes at this marker is:

Pr(CC)=p²=0.26, Pr(CT)=2pq=0.50, and Pr(TT)=q²=0.24

The average population risk relative to genotype TT (which is defined to have a risk of one) is:

R=0.26×1.28+0.50×1.13+0.24×1=1.14

Therefore, the risk relative to the general population (RR) for individuals who have one of the following genotypes at this marker is:

RR(CC)=1.28/1.14=1.12, RR(CT)=1.13/1.14=0.99, RR(TT)=1/1.14=0.88.

Risk for other markers described herein (e.g., rs10934853, rs16902094 and rs445114) may be described in an analogous fashion. Determining risk compared with non-carriers of the risk allele C will of course give higher values of RR.

Combining the risk from multiple markers. When genotypes of many SNP variants are used to estimate the risk for an individual, unless otherwise stated, a multiplicative model for risk can be assumed. This means that the combined genetic risk relative to the population is calculated as the product of the corresponding estimates for individual markers, e.g. for two markers g1 and g2:

RR(g1,g2)=RR(g1)RR(g2)

The underlying assumption is that the risk factors occur and behave independently, i.e. that the joint conditional probabilities can be represented as products:

Pr(A|g1,g2)=Pr(A|g1)Pr(A|g2)/Pr(A) and Pr(g1,g2)=Pr(g1)Pr(g2)

Obvious violations to this assumption are markers that are closely spaced on the genome, i.e. in linkage disequilibrium such that the concurrence of two or more risk alleles is correlated. In such cases, we can use so called haplotype modeling where the odds-ratios are defined for all allele combinations of the correlated SNPs.

As is in most situations where a statistical model is utilized, the model applied is not expected to be exactly true since it is not based on an underlying bio-physical model. However, the multiplicative model has so far been found to fit the data adequately, i.e. no significant deviations are detected for many common diseases for which many risk variants have been discovered.

A number of genetic markers in different genomic locations have been found to be associated with prostate cancer, as shown in Table 7, in addition to the markers shown herein to be associated with risk of prostate cancer. It can be useful to estimate genetic risk of prostate cancer for combinations of such markers, optionally including any one, or a combination of, the markers described herein. Determining risk for multiple markers captures a greater percentage of the genetic risk of prostate cancer in the population. For example, by combining risk for 22 prostate cancer risk variants typed in the Icelandic population, carriers belonging to the top 1.3% of the risk distribution have a risk of developing the disease that is more than 2.5 times greater than the population average risk estimates (see Table 7). For these individuals this corresponds to a lifetime risk of over 25% of being diagnosed with prostate cancer, compared with a population average life time risk of about 10% in Iceland.

As an example of how combined risk may be estimated, an individual who has the following genotypes at 8 markers associated with risk of prostate cancer along with the risk relative to the population at each marker:

| | | | |
|---|---|---|---|
| rs2710646 | AA | Calculated risk: | RR(AA) = 1.25 |
| rs16901979 | CC | Calculated risk: | RR(CC) = 0.96 |
| rs1447295 | AC | Calculated risk: | RR(AC) = 1.39 |
| rs6983267 | GT | Calculated risk: | RR(GT) = 0.99 |
| rs7947353 | AA | Calculated risk: | RR(AA) = 1.19 |
| rs1859962 | GG | Calculated risk: | RR(GG) = 1.21 |
| rs4430796 | GG | Calculated risk: | RR(GG) = 0.82 |
| rs5945572 | AA | Calculated risk: | RR(AA) = 1.14 |

Combined, the overall risk relative to the population for this individual is: 1.25×0.96×1.39×0.99×1.19×1.21×0.82×1.14=2.22.

We can combine risk for the markers described herein (e.g., rs16902094, rs8102476, rs445114 and rs10934853, or surrogate markers in linkage disequilibrium with any one of these markers), or any combination of the markers described herein with other markers conferring risk of prostate cancer in an analogous fashion. Calculated combined risk can thus be obtained for any combination of such markers.

In certain embodiments, combined risk of prostate cancer is determined for any combination of two or more markers selected from the group consisting of rs2710646 on chromosome 2p15, rs2660753 on chromosome 3p12, rs401681 on chromosome 5p15, rs9364554 on chromosome 6q25, rs10486567 on chromosome 7p15, rs6465657 on chromosome 7q21, rs1447295 on chromosome 8q24, rs16901979 on chromosome 8q24, rs6983267 on chromosome 8q24, rs1571801 on chromosome 9q33, rs10993994 on chromosome 10q11, rs4962416 on chromosome 10q26, rs10896450 on chromosome 11q13, rs4430796 on chromosome 17q12, rs11649743 on chromosome 17q12, rs1859962 on chromosome 17q24.3, rs2735839 on chromosome 19q13.33, rs9623117 on chromosome 22q13, rs5945572 on chromosome Xp11, rs10934853 on chromosome 3q21, rs16902094 on chromosome 8q24, rs445114 on chromosome 8q24 and rs8102476 on chromosome 19q13. Alternatively, any surrogate markers for these markers can be used in such risk assessment. For example, rs721048 is a surrogate marker for rs2710646; rs10896449 and rs7931342 are surrogate markers for rs10896450, and rs5945619 is a surrogate marker for rs5945572.

In certain embodiments, combined risk is determined for 3 or more markers. In certain other embodiments, combined risk is determined for 4 or more markers. In certain other embodiments, combined risk is determined for 5 or more markers. In certain other embodiments, combined risk is determined for 6 or more markers. In certain other embodiments, combined risk is determined for 7 or more markers. In certain other embodiments, combined risk is determined for 8 or more markers. In certain other embodiments, combined risk is determined for 9 or more markers. In certain other embodiments, combined risk is determined for 10 or more markers, including 11 or more, 12 or more, 13 or more, 14 or more, 15 or more, 16 or more, 17 or more, 18 or more, 19 or more, 20 or more, 21 one or more, 22 two or more, 23 or more markers, 24 or more markers, 25 or more markers, 26 or more markers, 27 or more markers, or 28 or more markers.

In certain embodiments, combined risk is determined for no more than fifty markers. In certain embodiments, combined risk is determined for no more than thirty markers, no more than 25 markers, no more than 23 markers, no more than 22 markers, no more than 21 markers, no more than 20 markers, no more than 15 markers, or no more than 10 markers.

In certain embodiments, any one, or a combination of, the markers rs16902094, rs10934853, rs445114 and rs8102476, may be assessed in combination with any one marker, or a combination of markers, selected from the group consisting of rs2710646, rs2660753, rs401681, rs9364554, rs10486567, rs6465657, rs1447295, rs16901979, rs6983267, rs1571801, rs10993994, rs4962416, rs10896450, rs4430796, rs11649743, rs1859962, rs2735839, rs9623117, rs5945572, rs7127900, rs10896449, rs8102476, rs5759167, rs10207654, rs7679673, rs1512268, rs10505483, and rs10086908. For these markers, rs2710646 allele A, rs2660753 allele T, rs401681 allele C, rs9364554 allele T, rs10486567 allele G, rs6465657 allele C, rs1447295 allele A, rs16901979 allele A, rs6983267 allele G, rs1571801 allele A, rs10993994 allele T, rs4962416 allele C, rs10896450 allele G, rs4430796 allele A, rs11649743 allele G, rs1859962 allele G, rs2735839 allele G, rs9623117 allele C, rs5945572 allele A rs7127900 allele A, rs10896449 allele G, rs8102476 allele C, rs5759167 allele G, rs10207654 allele A, rs7679673 allele C, rs1512268 allele A, rs10505483 allele A, and rs10086908 allele T are indicative of increased susceptibility of prostate cancer, and the alternate allele is thus indicative of decreased susceptibility of prostate cancer.

In one preferred embodiment, combined risk is determined for any combination of two or more markers selected from the group consisting of rs2710646, rs16901979, rs1447295, rs6983267, rs7947353, rs1859962, rs4430796, rs5945572, rs16902094, rs16902104, rs8102476, rs445114, rs620861 and rs10934853. In another preferred embodiment, combined risk is determined for the group of markers consisting of rs2710646, rs16901979, rs1447295, rs6983267, rs7947353, rs1859962, rs4430796, rs5945572, rs16902094, rs8102476, rs445114 and rs10934853. In another preferred embodiment, combined risk is determined for the group of markers consisting of rs2710646, rs16901979, rs1447295, rs6983267, rs7947353, rs1859962, rs4430796, rs5945572 and rs16902094.

### Adjusted Life-Time Risk

The lifetime risk of an individual is derived by multiplying the overall genetic risk relative to the population with the average life-time risk of the disease in the general population of the same ethnicity and gender and in the region of the individual's geographical origin. As there are usually several epidemiologic studies to choose from when defining the general population risk, we will pick studies that are well-powered for the disease definition that has been used for the genetic variants.

For example, if the overall genetic risk relative to the population for a disease is 1.8 for a white male, and if the average life-time risk of the disease for individuals of his demographic is 20%, then the adjusted lifetime risk for him is 20%×1.8=36%.

Note that since the average RR for a population is one, this multiplication model provides the same average adjusted life-time risk of the disease. Furthermore, since the actual life-time risk cannot exceed 100%, there must be an upper limit to the genetic RR.

### Risk Assessment for Prostate Cancer

As described herein, certain polymorphic markers and haplotypes comprising such markers are found to be useful for risk assessment of prostate cancer. Risk assessment can involve the use of the markers for determining a susceptibility to prostate cancer. Particular alleles of polymorphic markers (e.g., SNPs) are found more frequently in individuals with prostate cancer, than in individuals without diagnosis of prostate cancer. Therefore, these marker alleles have predictive value for detecting prostate cancer, or a susceptibility to prostate cancer, in an individual. Tagging markers in linkage disequilibrium with at-risk variants (or protective variants) described herein can be used as surrogates for these markers (and/or haplotypes). Such surrogate markers can be located within a particular haplotype block or LD block. Such surrogate markers can also sometimes be located outside the physical boundaries of such a haplotype block or LD block, either in close vicinity of the LD block/haplotype block, but possibly also located in a more distant genomic location.

Long-distance LD can for example arise if particular genomic regions (e.g., genes) are in a functional relationship. For example, if two genes encode proteins that play a role in a shared metabolic pathway, then particular variants in one gene may have a direct impact on observed variants for the other gene. Let us consider the case where a variant in one gene leads to increased expression of the gene product. To counteract this effect and preserve overall flux of the particular pathway, this variant may have led to selection of one (or more) variants at a second gene that confers decreased expression levels of that gene. These two genes may be located in different genomic locations, possibly on different chromosomes, but variants within the genes are in apparent LD, not because of their shared physical location within a region of high LD, but rather due to evolutionary forces. Such LD is also contemplated and within scope of the present invention. The skilled person will appreciate that many other scenarios of functional gene-gene interaction are possible, and the particular example discussed here represents only one such possible scenario.

Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high as or possibly even higher than the at-risk variant. The at-risk variant identified may not be the functional variant itself, but is in this instance in linkage disequilibrium with the true functional variant. The functional variant may for example be a tandem repeat, such as a minisatellite or a microsatellite, a transposable element (e.g., an Alu element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and genotype surrogate markers in linkage disequilibrium with the markers and/or haplotypes as described herein. The tagging or surrogate markers in LD with the at-risk variants detected, also have predictive value for detecting association to the disease, or a susceptibility to the disease, in an individual. These tagging or surrogate markers that are in LD with the markers of the present invention can also include other markers that distinguish among haplotypes, as these similarly have predictive value for detecting susceptibility to the particular disease.

The present invention can in certain embodiments be practiced by assessing a sample comprising genomic DNA from an individual for the presence of variants described herein to be associated with prostate cancer. Such assessment typically steps that detect the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of prostate cancer. Detecting particular alleles of polymorphic markers can in certain embodiments be done by obtaining nucleic acid sequence data about a particular human individual, which identifies at least one allele of at least one polymorphic marker. Different alleles of the at least one marker are associated with different susceptibility to the disease in humans. Obtaining nucleic acid sequence data can comprise nucleic acid sequence at a single nucleotide position, which is sufficient to identify alleles at SNPs. The nucleic acid sequence data can also comprise sequence at any other number of nucleotide positions, in particular for genetic markers that comprise multiple nucleotide positions, and can be anywhere from two to hundreds of thousands, possibly even millions, of nucleotides (in particular, in the case of copy number variations (CNVs)).

In certain embodiments, the invention can be practiced utilizing a dataset comprising information about the genotype status of at least one polymorphic marker associated with a disease (or markers in linkage disequilibrium with at least one marker associated with the disease). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with the disease. A positive result for a variant (e.g., marker allele) associated with the disease, is indicative of the individual from which the dataset is derived is at increased susceptibility (increased risk) of the disease.

In certain embodiments of the invention, a polymorphic marker is correlated to a disease by referencing genotype data for the polymorphic marker to a look-up table that comprises correlations between at least one allele of the polymorphism and the disease. In some embodiments, the table comprises a correlation for one polymorphism. In other embodiments, the table comprises a correlation for a plurality of polymorphisms. In both scenarios, by referencing to a look-up table that gives an indication of a correlation between a marker and the disease, a risk for the disease, or a susceptibility to the disease, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

The markers described herein may be useful for risk assessment and diagnostic purposes, either alone or in combination. Results of prostate cancer risk based on the markers described herein can also be combined with data for other genetic markers or risk factors for prostate cancer, to establish overall risk, as illustrated and described in the above. Thus, even in cases where the increase in risk by individual markers is relatively modest, e.g. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease.

Thus, in certain embodiments of the invention, a plurality of variants (genetic markers, biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to prostate cancer. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses or other methods known to the skilled person, may subsequently be used to determine the overall risk conferred based on the genotype status at the multiple loci. Assessment of risk based on such analysis may subsequently be used in the methods, uses and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined, as discussed in the above. Such markers and/or haplotypes may in those cases be also used as surrogate markers and/or haplotypes for the markers and/or haplotypes physically residing within the haplotype block as defined. As a consequence, markers and haplotypes in LD (typically characterized by inter-marker r² values of greater than 0.1, such as r² greater than 0.2, including r² greater than 0.3, also including markers correlated by values for r² greater than 0.4) with the markers and haplotypes of the present invention are also within the scope of the invention, even if they are physically located beyond the boundaries of the haplotype block as defined. This includes markers that are described herein but may also include other markers that are in LD with one or more of the these markers.

For the SNP markers described herein, the opposite allele to the allele found to be in excess in patients (at-risk allele) is found in decreased frequency in prostate cancer. These markers and haplotypes in LD and/or comprising such markers, are thus protective for prostate cancer, i.e. they confer a decreased risk or susceptibility of individuals carrying these markers and/or haplotypes developing prostate cancer.

Certain variants of the present invention, including certain haplotypes comprise, in some cases, a combination of various genetic markers, e.g., SNPs and microsatellites. Detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype found to be associated with prostate cancer, is one in which the marker allele or haplotype is more frequently present in an individual at risk for prostate cancer (affected), compared to the frequency of its presence in a healthy individual (control), or in randombly selected individual from the population, wherein the presence of the marker allele or haplotype is indicative of a susceptibility to prostate cancer. In other embodiments, at-risk markers in linkage disequilibrium with one or more markers shown herein to be associated with prostate cancer are tagging markers that are more frequently present in an individual at risk for prostate cancer (affected), compared to the frequency of their presence in a healthy individual (control) or in a randomly selected individual from the population, wherein the presence of the tagging markers is indicative of increased susceptibility to prostate cancer. In a further embodiment, at-risk markers alleles (i.e. conferring increased susceptibility) in linkage disequilibrium with one or more markers found to be associated with prostate cancer, are markers comprising one or more allele that is more frequently present in an individual at risk for prostate cancer, compared to the frequency of their presence in a healthy individual (control), wherein the presence of the markers is indicative of increased susceptibility to prostate cancer.

### Study Population

In a general sense, the methods and kits of the invention can be utilized from samples containing nucleic acid material (DNA or RNA) from any source and from any individual, or from genotype data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers and/or haplotypes in individuals who are members of a target population. Such a target population is in one embodiment a population or group of individuals at risk of developing the disease, based on other genetic factors, biomarkers (e.g., PSA), biophysical parameters, or general health and/or lifestyle parameters (e.g., history of prostate cancer or related cancer, previous diagnosis of prostate cancer, family history of prostate cancer).

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of prostate cancer in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Styrkarsdottir, U., et al. N Engl J Med Apr. 29, 2008 (Epub ahead of print); Thorgeirsson, T., et al. Nature 452:638-42 (2008); Gudmundsson, J., et al. Nat Genet. 40:281-3 (2008); Stacey, S, N., et al., Nat Genet. 39:865-69 (2007); Helgadottir, A., et al., Science 316:1491-93 (2007); Steinthorsdottir, V., et al., Nat Genet. 39:770-75 (2007); Gudmundsson, et al., Nat Genet. 39:631-37 (2007); Frayling, T M, Nature Reviews Genet 8:657-662 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including populations from Africa and Asia.

It is thus believed that the markers of the present invention found to be associated with prostate cancer will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portuguese, Italian, Polish, Bulgarian, Slavic, Serbian, Bosnian, Czech, Greek and Turkish populations.

In certain embodiments, the invention relates to populations that include black African ancestry such as populations comprising persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. In another example, self-reported persons of black African ancestry may have at least one parent of black African ancestry or at least one grandparent of black African ancestry.

The racial contribution in individual subjects may also be determined by genetic analysis.

Genetic analysis of ancestry may be carried out using unlinked microsatellite markers such as those set out in Smith et al. (Am J Hum Genet 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The person skilled in the art will appreciate and understand that the variants described herein in general do not, by themselves, provide an absolute identification of individuals who will develop a particular disease. The variants described herein do however indicate increased and/or decreased likelihood that individuals carrying the at-risk or protective variants of the invention will develop symptoms associated with prostate cancer. This information is however extremely valuable in itself, as outlined in more detail in the below, as it can be used to, for example, initiate preventive measures at an early stage, perform regular physical and/or mental exams to monitor the progress and/or appearance of symptoms, or to schedule exams at a regular interval to identify the condition in question, so as to be able to apply treatment at an early stage.

The knowledge of a genetic variant that confers a risk of developing prostate cancer offers the opportunity to apply a genetic-test to distinguish between individuals with increased risk of developing the cancer (i.e. carriers of the at-risk variant) and those with decreased risk of developing the cancer (i.e. carriers of the protective variant, or non-carriers of the at-risk variant). The core values of genetic testing, for individuals belonging to both of the above mentioned groups, are the possibilities of being able to diagnose the cancer at an early stage and provide information to the clinician about prognosis/aggressiveness of the disease in order to be able to apply the most appropriate treatment. For example, the application of a genetic test for prostate cancer (including aggressive or high Gleason grade prostate cancer, less aggressive or low Gleason grade prostate cancer)) can provide an opportunity for the detection of the cancer at an earlier stage which may lead to the application of therapeutic measures at an earlier stage, and thus can minimize the deleterious effects of the symptoms and serious health consequences conferred by cancer. Some advantages of genetic tests for prostate cancer include:

### 1. To Aid Early Detection

The application of a genetic test for prostate cancer can provide an opportunity for the detection of the disease at an earlier stage which leads to higher cure rates, if found locally, and increases survival rates by minimizing regional and distant spread of the tumor. For prostate cancer, a genetic test will most likely increase the sensitivity and specificity of the already generally applied Prostate Specific Antigen (PSA) test and Digital Rectal Examination (DRE). This can lead to lower rates of false positives (thus minimize unnecessary procedures such as needle biopsies) and false negatives (thus increasing detection of occult disease and minimizing morbidity and mortality due to PCA).

### 2. To Determine Aggressiveness

Genetic testing can provide information about pre-diagnostic prognostic indicators and enable the identification of individuals at high or low risk for aggressive tumor types that can lead to modification in screening strategies. For example, an individual determined to be a carrier of a high risk allele for the development of aggressive prostate cancer will likely undergo more frequent PSA testing, examination and have a lower threshold for needle biopsy in the presence of an abnormal PSA value.

Furthermore, identifying individuals that are carriers of high or low risk alleles for aggressive tumor types will lead to modification in treatment strategies. For example, if prostate cancer is diagnosed in an individual that is a carrier of an allele that confers increased risk of developing an aggressive form of prostate cancer, then the clinician would likely advise a more aggressive treatment strategy such as a prostatectomy instead of a less aggressive treatment strategy.

As is known in the art, Prostate Specific Antigen (PSA) is a protein that is secreted by the epithelial cells of the prostate gland, including cancer cells. An elevated level in the blood indicates an abnormal condition of the prostate, either benign or malignant. PSA is used to detect potential problems in the prostate gland and to follow the progress of prostate cancer therapy. PSA levels above 4 ng/ml are indicative of the presence of prostate cancer (although as known in the art and described herein, the test is neither very specific nor sensitive).

In one embodiment, the method of the invention is performed in combination with (either prior to, concurrently or after) a PSA assay. In a particular embodiment, the presence of an at-risk marker or haplotype, in conjunction with the subject having a PSA level greater than 4 ng/ml, is indicative of a more aggressive prostate cancer and/or a worse prognosis. As described herein, particular markers and haplotypes are associated with high Gleason (i.e., more aggressive) prostate cancer. In another embodiment, the presence of a marker or haplotype, in a patient who has a normal PSA level (e.g., less than 4 ng/ml), is indicative of a high Gleason (i.e., more aggressive) prostate cancer and/or a worse prognosis. A “worse prognosis” or “bad prognosis” occurs when it is more likely that the cancer will grow beyond the boundaries of the prostate gland, metastasize, escape therapy and/or kill the host.

In one embodiment, the presence of a marker or haplotype is indicative of a predisposition to a somatic rearrangement (e.g., one or more of an amplification, a translocation, an insertion and/or deletion) in a tumor or its precursor. The somatic rearrangement itself may subsequently lead to a more aggressive form of prostate cancer (e.g., a higher histologic grade, as reflected by a higher Gleason score or higher stage at diagnosis, an increased progression of prostate cancer (e.g., to a higher stage), a worse outcome (e.g., in terms of morbidity, complications or death)). As is known in the art, the Gleason grade is a widely used method for classifying prostate cancer tissue for the degree of loss of the normal glandular architecture (size, shape and differentiation of glands). A grade from 1-5 is assigned successively to each of the two most predominant tissue patterns present in the examined tissue sample and are added together to produce the total or combined Gleason grade (scale of 2-10). High numbers indicate poor differentiation and therefore more aggressive cancer.

Aggressive prostate cancer is cancer that grows beyond the prostate, metastasizes and eventually kills the patient. As described herein, one surrogate measure of aggressiveness is a high combined Gleason grade. The higher the grade on a scale of 2-10 the more likely it is that a patient has aggressive disease.

The present invention furthermore relates to risk assessment for prostate cancer and colorectal cancer, including diagnosing whether an individual is at risk for developing prostate cancer and/or colorectal cancer. The polymorphic markers of the present invention can be used alone or in combination, as well as in combination with other factors, including other genetic risk factors or biomarkers, for risk assessment of an individual for prostate cancer and/or colorectal cancer. Certain factors known to affect the predisposition of an individual towards developing risk of developing common disease, including prostate cancer and/or colorectal cancer are known to the person skilled in the art and can be utilized in such assessment. These include, but are not limited to, age, gender, smoking status, family history of cancer, previously diagnosed cancer, colonic adenomas, chronic inflammatory bowel disease and diet. Methods known in the art can be used for such assessment, including multivariate analyses or logistic regression.

### Methods

Methods for disease risk assessment and risk management are described herein and are encompassed by the invention. The invention also encompasses methods of assessing an individual for probability of response to a therapeutic agents, methods for predicting the effectiveness of a therapeutic agents, nucleic acids, polypeptides and antibodies and computer-implemented functions. Kits for use in the various methods presented herein are also encompassed by the invention.

### Diagnostic and Screening Methods

In certain embodiments, the present invention pertains to methods of diagnosing, or aiding in the diagnosis of, prostate cancer or a susceptibility to prostate cancer, by detecting particular alleles at genetic markers that appear more frequently in prostate cancer subjects or subjects who are susceptible to prostate cancer. In certain other embodiments, the invention is a method of determining a susceptibility to prostate cancer by detecting and/or assessing at least one allele of at least one polymorphic marker (e.g., the markers described herein). In other embodiments, the invention relates to a method of determining a susceptibility to prostate cancer by detecting at least one allele of at least one polymorphic marker. The present invention describes methods whereby detection of particular alleles of particular markers or haplotypes is indicative of a susceptibility to prostate cancer. Such prognostic or predictive assays can also be used to determine prophylactic treatment of a subject prior to the onset of symptoms of prostate cancer.

The present invention pertains in some embodiments to methods of clinical applications of diagnosis, e.g., diagnosis performed by a medical professional. In other embodiments, the invention pertains to methods of diagnosis or methods of determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications. The diagnostic application of disease-associated alleles as described herein, can thus for example be performed by the individual, through analysis of his/her genotype data, by a health professional based on results of a clinical test, or by a third party, including the genotype service provider. The third party may also be service provider who interprets genotype information from the customer to provide service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human condition, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for a heterozygous carrier of an at-risk variant for a particular disease or trait (such as prostate cancer). The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

In addition, in certain other embodiments, the present invention pertains to methods of determining a decreased susceptibility to prostate cancer, by detecting particular genetic marker alleles or haplotypes that appear less frequently in prostate cancer patients than in individual not diagnosed with prostate cancer or in the general population.

As described and exemplified herein, particular marker alleles or haplotypes are associated with prostate cancer. In one embodiment, the marker allele or haplotype is one that confers a significant risk or susceptibility to prostate cancer. In another embodiment, the invention relates to a method of determining a susceptibility to prostate cancer in a human individual, the method comprising determining the presence or absence of at least one allele of at least one polymorphic marker in a nucleic acid sample obtained from the individual. In another embodiment, the invention pertains to methods of determining a susceptibility to prostate cancer in a human individual, by screening for certain marker alleles or haplotypes. In certain embodiments, the marker allele or haplotype is more frequently present in a subject having, or who is susceptible to, prostate cancer (affected), as compared to the frequency of its presence in a healthy subject (control, such as population controls). In certain embodiments, the significance of association of the at least one marker allele or haplotype is characterized by a p value <0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to prostate cancer. These diagnostic methods involve determining whether particular alleles or haplotypes that are associated with risk of prostate cancer are present in particular individuals. The haplotypes described herein include combinations of alleles at various genetic markers (e.g., SNPs, microsatellites or other genetic variants). The detection of particular genetic marker alleles can be performed by a variety of methods described herein and/or known in the art. For example, genetic markers can be detected at the nucleic acid level (e.g., by direct nucleotide sequencing, or by other genotyping means known to the skilled in the art) or at the amino acid level if the genetic marker affects the coding sequence of a protein (e.g., by protein sequencing or by immunoassays using antibodies that recognize such a protein). The marker alleles or haplotypes of the present invention correspond to fragments of a genomic segments (e.g., genes) associated with prostate cancer. Such fragments encompass the DNA sequence of the polymorphic marker or haplotype in question, but may also include DNA segments in strong LD (linkage disequilibrium) with the marker or haplotype. In one embodiment, such segments comprises segments in LD with the marker or haplotype as determined by a value of r² greater than 0.1 and/or |D′|>0.8). In another embodiment, the segments are in LD with the marker or haplotype as determined by a value of r² of greater than 0.2.

In one embodiment, determination of a susceptibility to prostate cancer can be accomplished using hybridization methods. (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies and methods for genotyping particular polymorphic markers.

To determine a susceptibility to prostate cancer, a hybridization sample can be formed by contacting the test sample containing prostate cancer-associated nucleic acid, such as a genomic dna sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of LD Block C19, LD Block C03, LD Block C08A and/or LD Block C08B, as described herein, optionally comprising at least one allele of a marker described herein, or at least one haplotype described herein, or the probe can be the complementary sequence of such a sequence. In a particular embodiment, the nucleic acid probe is a portion of the nucleotide sequence of LD Block C19, LD Block C03, LD Block C08A and/or LD Block C08B as described herein, optionally comprising at least one allele of a marker described herein, or at least one allele of one polymorphic marker or haplotype comprising at least one polymorphic marker described herein, or the probe can be the complementary sequence of such a sequence. The nucleic acid probe may also comprise all or a portion of the nucleotide sequence of a nucleotide with sequence as set forth in any one of SEQ ID NO:1-978 herein, or it can be the complement of such a sequence. The probe may optionally comprise at least one polymorphic marker as described herein. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to prostate cancer.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

Alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with prostate cancer. Hybridization of the PNA probe is thus diagnostic for prostate cancer or a susceptibility to prostate cancer.

In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis, for example by using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s). Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes. Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid that contains a polymorphic marker or haplotype, and the presence of specific alleles can then be detected directly by sequencing the polymorphic site (or multiple polymorphic sites in a haplotype) of the genomic DNA in the sample.

In another embodiment, arrays of oligonucleotide probes that are complementary to target nucleic acid sequence segments from a subject, can be used to identify particular alleles at polymorphic sites. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of prostate cancer or a determination of a susceptibility to prostate cancer can be made by examining expression and/or composition of a polypeptide encoded by a nucleic acid associated with prostate cancer in those instances where the genetic marker(s) or haplotype(s) of the present invention result in a change in the composition or expression of the polypeptide. Thus, determination of a susceptibility to prostate cancer can be made by examining expression and/or composition of one of these polypeptides, or another polypeptide encoded by a nucleic acid associated with prostate cancer, in those instances where the genetic marker or haplotype of the present invention results in a change in the composition or expression of the polypeptide. The haplotypes and markers of the present invention that show association to prostate cancer may play a role through their effect on one or more of such nearby genes. In certain embodiments, markers or haplotype exerts its effect on the composition or expression on a gene selected from the group consisting of the EEFSEC gene, the SEC61A1 gene, the RUVBL1 gene, and the PPP1R14A gene. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

Thus, in another embodiment, the variants (markers or haplotypes) presented herein affect the expression of a particular gene. It is well known that regulatory element affecting gene expression may be located far away, even as far as tenths or hundreds of kilobases away, from the promoter region of a gene. By assaying for the presence or absence of at least one allele of at least one polymorphic marker of the present invention, it is thus possible to assess the expression level of such nearby genes. It is thus contemplated that the detection of the markers or haplotypes of the present invention can be used for assessing expression for one or more genes whose expression is affected by the allelic and/or haplotype status at these markers and/or haplotypes (e.g., a gene selected from the group consisting of the EEFSEC gene, the SEC61A1 gene, the RUVBL1 gene, and the PPP1R14A gene).

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a particular nucleic acid. An alteration in expression of a polypeptide encoded by the nucleic acid can be, for example, an alteration in the quantitative polypeptide expression (i.e., the amount of polypeptide produced). An alteration in the composition of a polypeptide encoded by the nucleic acid is an alteration in the qualitative polypeptide expression (e.g., expression of a mutant polypeptide or of a different splicing variant). In one embodiment, diagnosis of a susceptibility to prostate cancer is made by detecting a particular splicing variant encoded by a nucleic acid associated with prostate cancer, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who is not affected by, and/or who does not have a susceptibility to, prostate cancer. In one embodiment, the control sample is from a subject that does not possess a marker allele or haplotype associated with prostate cancer, as described herein. Similarly, the presence of one or more different splicing variants in the test sample, or the presence of significantly different amounts of different splicing variants in the test sample, as compared with the control sample, can be indicative of a susceptibility to prostate cancer. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with prostate cancer can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of a polypeptide in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, determination of a susceptibility to prostate cancer is made by detecting at least one marker or haplotype of the present invention, in combination with an additional protein-based, RNA-based or DNA-based assay.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with prostate cancer, means for analyzing the nucleic acid sequence of a nucleic acid associated with prostate cancer, means for analyzing the amino acid sequence of a polypeptide encoded by a nucleic acid associated with prostate cancer, etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., dna polymerase). Additionally, kits can provide reagents for assays to be used in combination with the methods of the present invention, e.g., reagents for use with other diagnostic assays for prostate cancer.

In one embodiment, the invention pertains to a kit for assaying a sample from a subject to detect a susceptibility to prostate cancer in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with prostate cancer risk. In one such embodiment, the polymorphism is selected from the group consisting of the markers described herein to be associated with risk of prostate cancer, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are associated with risk of prostate cancer. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers rs445114, rs8102476, rs10934853 and rs16902094, and markers in linkage disequilibrium therewith. In another embodiment, the marker or haplotype to be detected comprises one or more markers, two or more markers, three or more markers, four or more markers or five or more markers selected from the group consisting of the markers set forth in Tables 8, 9, 10, 11, 17, 18, 19 and 20 herein. In another embodiment, the marker or haplotype to be detected comprises at least one marker from the group of markers in strong linkage disequilibrium, as defined by values of r² greater than 0.2, to at least one of the group of markers listed in Tables 8, 9, 10, 11, 17, 18, 19 and 20 herein. In another embodiment, the marker or haplotype to be detected is selected from the group consisting of rs445114, rs8102476, rs10934853, rs16902094, rs16902104, and rs620861.

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one such embodiment, determination of the presence of the marker or haplotype is indicative of a susceptibility (increased susceptibility or decreased susceptibility) to prostate cancer. In another embodiment, determination of the presence of the marker or haplotype is indicative of response to a therapeutic agent for prostate cancer. In another embodiment, the presence of the marker or haplotype is indicative of prostate cancer prognosis. In yet another embodiment, the presence of the marker or haplotype is indicative of progress of prostate cancer treatment. Such treatment may include intervention by surgery, medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or rnai molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit. In certain embodiments, the kit further comprises a collection of data comprising correlation data between the polymorphic markers assessed by the kit and susceptibility to prostate cancer and/or colorectal cancer.

### Therapeutic Agents

The variants (markers and/or haplotypes) disclosed herein to confer increased risk of prostate cancer can also be used to identify novel therapeutic targets for prostate cancer. For example, genes containing, or in linkage disequilibrium with, one or more of these variants, or their products, as well as genes or their products that are directly or indirectly regulated by or interact with these variant genes or their products, can be targeted for the development of therapeutic agents to treat prostate cancer, or prevent or delay onset of symptoms associated with prostate cancer. Therapeutic agents may comprise one or more of, for example, small non-protein and non-nucleic acid molecules, proteins, peptides, protein fragments, nucleic acids (dna, rna), pna (peptide nucleic acids), or their derivatives or mimetics which can modulate the function and/or levels of the target genes or their gene products.

The nucleic acids and/or variants described herein, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a nucleotide segment of the gene (e.g., the EEFSEC gene, the SEC61A1 gene, the RUVBL1 gene, or the PPP1R14A gene). Antisense nucleotides can be from 5-500 nucleotides in length, including 5-200 nucleotides, 5-100 nucleotides, 10-50 nucleotides, and 10-30 nucleotides. In certain preferred embodiments, the antisense nucleotides is from 14-50 nucleotides in length, including 14-40 nucleotides and 14-30 nucleotides. In certain such embodiments, the antisense nucleotide is capable of binding to a nucleotide segment with sequence as set forth in any one of SEQ ID NO:1-978.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

A genetic defect leading to increased predisposition or risk for development of a disease, such as prostate cancer, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat prostate cancer. In certain embodiments, such methods include assaying the ability of an agent or compound to modulate the activity and/or expression of a nucleic acid that includes at least one of the variants (markers and/or haplotypes) of the present invention, or the encoded product of the nucleic acid. This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating prostate cancer can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or therapeutic method acts on the body, or the way in which the body metabolizes the therapeutic agent.

Accordingly, in one embodiment, the presence of a particular allele at a polymorphic site or haplotype is indicative of a different response, e.g. a different response rate, to a particular treatment modality for prostate cancer. This means that a patient diagnosed with prostate cancer, and carrying a certain allele at a polymorphic or haplotype of the present invention (e.g., the at-risk and protective alleles and/or haplotypes of the invention) would respond better to, or worse to, a specific therapeutic, drug and/or other therapy used to treat the disease. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

In certain embodiments, assessment of the genetic status of an individual for genetic susceptibility markers for prostate cancer, e.g. the markers as described herein, is combined with assessment or assessment results for a biomarker indicative of prostate cancer, such as Prostate Specific Antigen (PSA).

The present invention also relates to methods of monitoring progress or effectiveness of a treatment for prostate cancer. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for prostate cancer as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention may be more likely to respond favorably to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with prostate cancer when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of lifestyle changes and administration of particular treatment, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

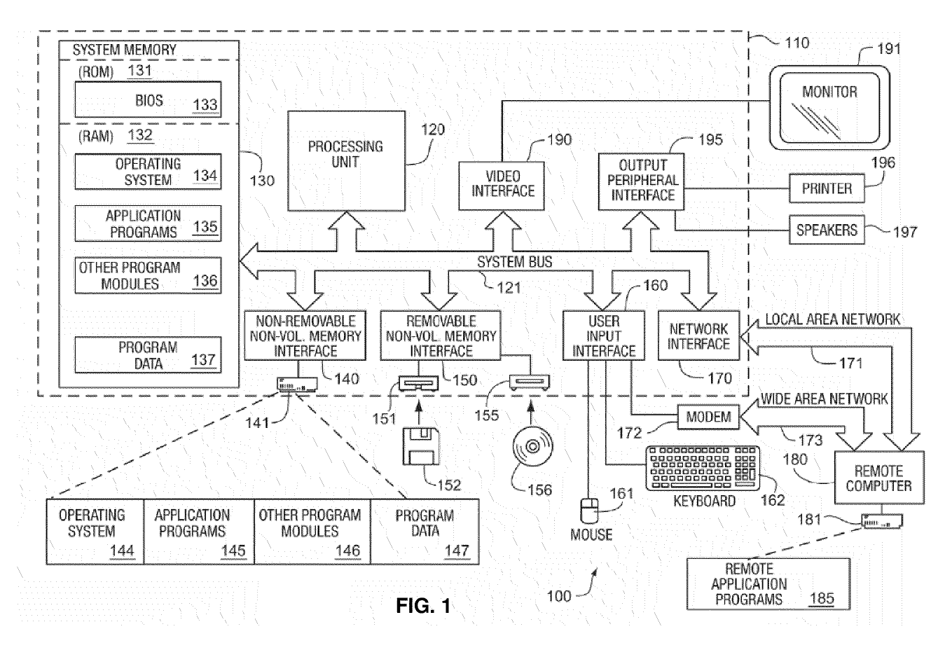

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, the invention relates to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a guardian of the individual, a health care provider or genetic analysis service provider), or for deriving information from the genotype data, e.g., by comparing the genotype data to information about genetic risk factors contributing to increased susceptibility to the prostate cancer, and reporting results based on such comparison.

In certain embodiments, computer-readable media suitably comprise capabilities of storing (i) identifier information for at least one polymorphic marker or a haplotype, as described herein; (ii) an indicator of the identity (e.g., presence or absence) of at least one allele of said at least one marker, or a haplotype, in individuals with prostate cancer; and (iii) an indicator of the risk associated with the marker allele or haplotype.

The markers and haplotypes described herein to be associated with increased susceptibility (increased risk) of prostate cancer, are in certain embodiments useful for interpretation and/or analysis of genotype data. Thus in certain embodiments, determination of the presence of an at-risk allele for prostate cancer, as shown herein, or determination of the presence of an allele at a polymorphic marker in LD with any such risk allele, is indicative of the individual from whom the genotype data originates is at increased risk of prostate cancer. In one such embodiment, genotype data is generated for at least one polymorphic marker shown herein to be associated with prostate cancer, or a marker in linkage disequilibrium therewith. The genotype data is subsequently made available to a third party, such as the individual from whom the data originates, his/her guardian or representative, a physician or health care worker, genetic counsellor, or insurance agent, for example via a user interface accessible over the internet, together with an interpretation of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk variants in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S, and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of any one of LD Block C19, LD Block C03, LD Block C08A, and LD Block C08B, or a nucleotide sequence comprising, or consisting of, the complement of the nucleotide sequence of any one of LD Block C19, LD Block C03, LD Block C08A and LD Block C08B, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length. In certain embodiments, the nucleic acid fragments are from about 15 to about 1000 nucleotides in length. In certain other embodiments, the nucleic acid fragments are from about 18 to about 100 nucleotides in length, from about 12 to about 50 nucleotides in length, from about 12 to about 40 nucleotides in length, or from about 12 to about 30 nucleotides in length.

The present invention further provides isolated nucleic acid molecules that contain a fragment or portion that hybridizes under highly stringent conditions to a nucleic acid that comprises, or consists of, the nucleotide sequence of any one of SEQ ID NO: 1-978, as described herein. The nucleic acid fragments can be from 10-600 nucleotides in length, such as from 10-500 nucleotides, 12-200 nucleotides, 12-100 nucleotides, 12-50 nucleotides and 12-30 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Antibodies

The invention also provides antibodies which bind to an epitope comprising either a variant amino acid sequence (e.g., comprising an amino acid substitution) encoded by a variant allele or the reference amino acid sequence encoded by the corresponding non-variant or wild-type allele. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. N. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurtZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorpic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein, in particular prostate cancer. Antibodies specific for a variant protein of the present invention that is encoded by a nucleic acid that comprises at least one polymorphic marker or haplotype as described herein can be used to screen for the presence of the variant protein, for example to screen for a predisposition to prostate cancer as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting example.

### Example 1

We and others have previously presented results from genome-wide association studies (GWAS) on prostate cancer reporting several common variants conferring risk of the disease (Gudmundsson, J. et al. Nat Genet 39, 631-7 (2007), Haiman, C. A. et al. Nat Genet 39, 638-44 (2007), Gudmundsson, J. et al. Nat Genet 39, 977-83 (2007), Eeles, R. A. et al. Nat Genet 40, 316-21 (2008), Thomas, G. et al. Nat Genet 40, 310-5 (2008), Gudmundsson, J. et al. Nat Genet 40, 281-3 (2008) and Yeager, M. et al. Nat Genet 39, 645-9 (2007)). By scrutinizing our Icelandic GWAS data, analyzing in-house follow-up and public data, as well as through fine-mapping work of previously published loci on 8q24.21, we identified four new variants conferring risk of prostate cancer.

The four new variants are: allele A of rs10934853 (rs10934853-A) located on 3q21.3, allele G of rs16902094 (rs16902094-G) on 8q24.21, allele T of rs445114 (rs445114-T) also on 8q24.21, and allele C of rs8102476 (rs8102476-C) located on 19q13.2. All SNPs, except rs16902094, are on the Illumina Hap317 chip used in the Icelandic GWAS. rs16902094 was discovered through Solexa re-sequencing of a 527 kb candidate region on 8q24 in pools of Icelandic cases and controls (see Methods). The allele specific odds ratios (ORs) of the four variants range between 1.09 and 1.28 in the Icelandic study group (P 6.4×10⁻³; Table 1). We proceeded to genotype all four SNPs in at least two out of five prostate cancer study groups (deCODE follow up groups) of European descent. These groups come from The Netherlands, Spain, Finland and the United States (US). When results were combined for SNPs successfully genotyped in these groups, they were significant for all loci, having an OR ranging from 1.07 to 1.20 (P<0.005). Combination of the Icelandic GWAS results and the data from the deCODE follow-up groups results in a genome wide significant association signals for rs16902094-G on 8q24 and rs8102476-C on 19q13.2 with an OR of 1.22 and 1.13, respectively (P<10⁻⁷), whereas rs10934853-A on 3q21.3 and rs445114-T on 8q24 have ORs of 1.10 and 1.17, not reaching genome-wide significance (P>10⁻⁷).

We tested if these association signals could be further confirmed by data released by the Cancer Genetics Markers of Susceptibility (CGEMS) study (Thomas, G. et al. Nat Genet 40, 310-5 (2008), Yeager, M. et al. Nat Genet 39, 645-9 (2007)) for five study groups (see Table 1) and in a paper by Duggan et al. (Duggan, D. et al. J Natl Cancer Inst 99, 1836-44 (2007)). Summary data were downloaded from the CGEMS web site for rs8102476 on 19q13.2, rs445114 on 8q24 and the SNPs rs4857841 on 3q21.3 and rs16902104 on 8q24, that are highly correlated with rs10934853 on 3q21.3 and rs16902094 on 8q24, discussed above (D′≧0.98 and r²≧0.96 according to CEU HapMap and/or Icelandic data). Duggan et al. published data for rs10934853 on 3q21.3 from a study on aggressive prostate cancer in the CAPS study population from Sweden (Duggan, D. et al. J Natl Cancer Inst 99, 1836-44 (2007)). When the public data were combined with the data discussed above, the results for rs10934853-A on 3q21.3 and rs445114-T on 8q24 became genome-wide significant, with an OR of 1.12 and 1.14, respectively (P) 4.7×10⁻¹°, and the results for rs8102476-C on 19q13.2 and rs16902094-G on 8q24 became even more significant, giving an OR of 1.12 and 1.21, respectively (P≦1.6×10⁻¹¹; Table 1). When inspected, a test of heterogeneity in the OR for all variants and all study groups showed a nominally significant heterogeneity (P=0.039) for the 3q21.3 locus, no significant difference was observed for the other three loci (P>0.1).

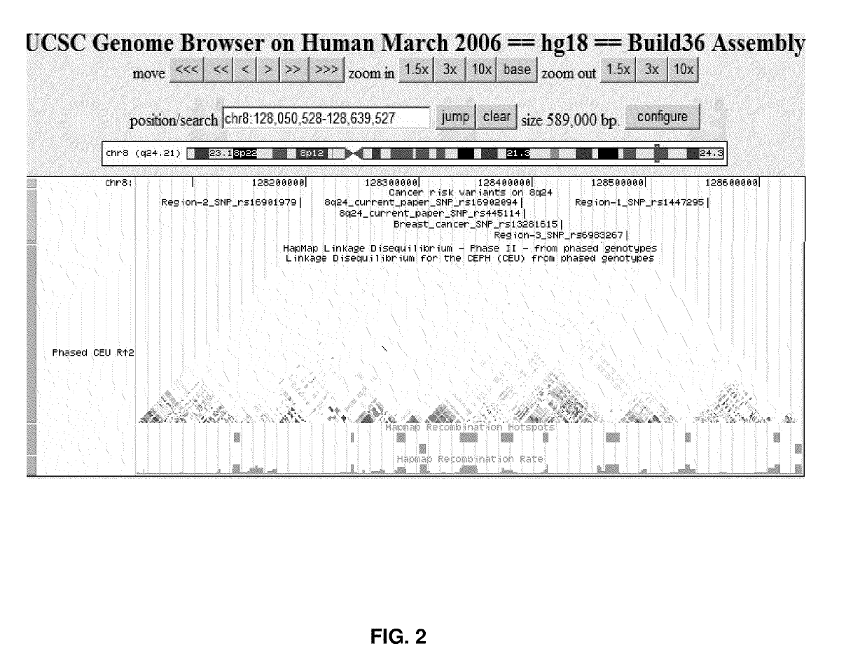

The two SNPs on 8q24, rs16902094 and rs445114, are located within the same linkage disequilibrium (LD) region but the correlation between them is very low (D′=1 and r²=0.07 according to Icelandic data) and the results for both remain significant after being adjusted for the other (Table 2). Of the previously published cancer variants on 8q24, only the breast cancer variant (rs13281615; Easton, D. F. et al. Nature 447, 1087-93 (2007)) is located within the same LD-region as the two new 8q24 SNPs and rs445114 is somewhat correlated with it (D′=0.76, r²=0.44; Table 3). However, both rs16902094 and rs445114 show very little correlation with any of the previously published prostate-(Gudmundsson, J. et al. Nat Genet 39, 631-7 (2007), Yeager, M. et al. Nat Genet 39, 645-9 (2007) and Amundadottir, L. T. et al. Nat Genet 38, 652-8 (2006)), colon-(Tomlinson, I. et al. Nat Genet 39, 984-8 (2007), Zanke, B. W. et al. Nat Genet 39, 989-94 (2007) and Heiman, C. A. et al. Nat Genet 39, 954-6 (2007)), or bladder cancer (Kiemeney, L. A. et al. Nat Genet 40, 1307-12 (2008)) risk variants on 8q24 (D′ ≦0.6 and r² ≦0.13; Table 3 and FIG. 2). The results in Iceland for rs16902094, rs445114 and the three previously published prostate cancer risk variants on 8q24, remain significant after being adjusted for each other (Table 4). Hence, rs16902094 and rs445114 can be added to the list of independent prostate cancer risk variants located on 8q24.

By computing the genotype specific ORs and inspecting the public data we found that the multiplicative model provides an adequate fit for all four loci in the study groups analyzed (Table 5).

The SNP rs10934853-A on 3q21.3 is located in the fourth intron of the EEFSEC gene, which is an elongation factor required for effective selenoprotein translation. Other RefSeq genes in the same LD region are SEC61A1 and RUVBL1. None of these genes has previously been directly implicated in prostate cancer. On 19q13.2, the SNP is located in a 178 kb LD-region with several annotated RefSeq genes. The closest one is PPP1R14A, a gene reported to be an inhibitor of smooth muscle myosin phosphatase. Similarly, the underlying biological perturbation on 8q24 has not yet been explained.

The four new loci reported here, add to the rapidly increasing number of prostate cancer susceptibility variants, identified through GWAS. In Table 6, we provide results from the Icelandic population for risk variants that are either widely considered or recently reported to confer risk of prostate cancer. The previously unpublished results from Iceland add support for susceptibility variants at several of these loci (Table 6). In a multi-variant analysis, using the multiplicative model for 22 risk variants, we combined the effect of all variants with an increased risk in the Icelandic population. Based on this analysis the estimated risk is more than 2.5-fold greater for the top 1.3% of the risk distribution, using the population average risk as a reference (Table 7). For these individuals this corresponds to a lifetime risk of over 25% of being diagnosed with prostate cancer, compared with a population average life time risk of about 10% in Iceland. These risk estimates are largely independent of family history (Eeles, R. A., et al., Nat Genet 40:316-21 (2008); Kote-Jarai, Z., et al., Cancer Epidemiol biomarkers Prev 17:2052-61 (2008)). Hence, the estimated risk for an individual can be increased further if history of prostate cancer is known among close relatives.

### Methods

Icelandic study population. Men diagnosed with prostate cancer were identified based on a nationwide list from the Icelandic Cancer Registry (ICR) (see URL below) that contained all 4,457 Icelandic prostate cancer patients diagnosed from Jan. 1, 1955, to Dec. 31, 2007. The Icelandic prostate cancer sample collection included 1,980 patients (diagnosed from December 1974 to December 2007) who were recruited from November 2000 until June 2008 out of the 2,283 affected individuals who were alive during the study period (a participation rate of about 86%). A total of 1,968 patients were included in the study with genotypes from a genome wide SNP genotyping effort, using the Infinium II assay method and the Sentrix HumanHap300 BeadChip (Illumina, San Diego, Calif., USA) and a Centaurus single track SNP genotyping assay (see Supplementary methods). The mean age at diagnosis for the consenting patients was 71 years (median 71 years) and the range was from 40 to 96 years, while the mean age at diagnosis was 73 years for all prostate cancer patients in the ICR. The median time from diagnosis to blood sampling was 2 years (range 0 to 26 years). In the present study, for all populations, aggressive prostate cancer is defined as: Gleason and/or T3 or higher and/or node positive and/or metastatic disease, while the less aggressive disease is defined as Gleason <7 and T2 or lower. The 35,470 controls (15,359 males (43.3%) and 20,111 females (56.7%)) used in this study consisted of individuals belonging to different genetic research projects at deCODE. The individuals have been diagnosed with common diseases of the cardio-vascular system (e.g. stroke or myocardial infraction), psychiatric and neurological diseases (e.g. schizophrenia, bipolar disorder), endocrine and autoimmune system (e.g. type 2 diabetes, asthma), malignant diseases (e.g. cancer of the breast, kidney, lung, thyroid or melanoma) as well as individuals randomly selected from the Icelandic genealogical database. No single disease project represented more than 6% of the total number of controls. The controls had a mean age of 84 years and the range was from 8 to 105 years. A linear regression analysis showed no correlation between allele frequency of SNPs discussed in the main text and year of birth among the Icelandic controls (P>0.1). The controls were absent from the nationwide list of prostate cancer patients according to the ICR. The DNA for both the Icelandic cases and controls was isolated from whole blood using standard methods.

The study was approved by the Data Protection Commission of Iceland and the National Bioethics Committee of Iceland. Written informed consent was obtained from all patients, relatives and controls. Personal identifiers associated with medical information and blood samples were encrypted with a third-party encryption system as previously described¹⁶.

### The Netherlands

The total number of Dutch prostate cancer cases used in this study was 1,100. The Dutch study population was comprised of two recruitment-sets of prostate cancer cases; Group-A was comprised of 390 hospital-based cases recruited from January 1999 to June 2006 at the Urology Outpatient Clinic of the Radboud University Nijmegen Medical Centre (RUNMC); Group-B consisted of 710 cases recruited from June 2006 to December 2006 through a population-based cancer registry held by the Comprehensive Cancer Centre IKO. Both groups were of self-reported European descent. The average age at diagnosis for patients in Group-A was 63 years (median 63 years) and the range was from 43 to 83 years. The average age at diagnosis for patients in Group-B was 65 years (median 66 years) and the range was from 43 to 75 years. The 2,021 control individuals (1,004 males and 1,017 females) were cancer free and were matched for age with the cases. They were recruited within a project entitled “The Nijmegen Biomedical Study”, in the Netherlands. This is a population-based survey conducted by the Department of Epidemiology and Biostatistics and the Department of Clinical Chemistry of the RUNMC, in which 9,371 individuals participated from a total of 22,500 age and sex stratified, randomly selected inhabitants of Nijmegen. Control individuals from the Nijmegen Biomedical Study were invited to participate in a study on gene-environment interactions in multifactorial diseases, such as cancer. All the 2,021 participants in the present study are of self-reported European descent and were fully informed about the goals and the procedures of the study. The study protocol was approved by the Institutional Review Board of Radboud University and all study subjects gave written informed consent.

### Spain

The Spanish study population used in this study consisted of 820 prostate cancer cases. The cases were recruited from the Oncology Department of Zaragoza Hospital in Zaragoza, Spain, from June 2005 to September 2007. All patients were of self-reported European descent. Clinical information including age at onset, grade and stage was obtained from medical records. The average age at diagnosis for the patients was 69 years (median 70 years) and the range was from 44 to 83 years. The 1,605 Spanish control individuals (737 males and 868 females) were approached at the University Hospital in Zaragoza, Spain, and the males were confirmed to be prostate cancer free before they were included in the study. Study protocols were approved by the Institutional Review Board of Zaragoza University Hospital. All subjects gave written informed consent.

### Chicago

The Chicago study population used consisted of 1,095 prostate cancer cases. The cases were recruited from the Pathology Core of Northwestern University's Prostate Cancer Specialized Program of Research Excellence (SPORE) from May 2002 to May 2007. The average age at diagnosis for the patients was 60 years (median 59 years) and the range was from 39 to 87 years. The 1,172 European American controls (781 males and 391 females) were recruited as healthy control subjects for genetic studies at the University of Chicago and Northwestern University Medical School, Chicago, US. All individuals from Chicago included in this report were of self-reported European descent. Study protocols were approved by the Institutional Review Boards of Northwestern University and the University of Chicago. All subjects gave written informed consent.

### Nashville

Study subjects were Americans of Northern European descent, ascertained with informed consent between 2002 and 2009 from Vanderbilt University Medical Center and from the VA Tennessee Valley Healthcare System (adjacent hospitals) with institutional review board oversight. Familial prostate cancer cases were ascertained at the time of treatment for the principal diagnosis of prostate cancer, and controls were ascertained at the time of routine preventative screening for prostate cancer. All prostate cancer probands included in the study were from pedigrees with a family history of prostate cancer (≧2 affected), and all control probands were from pedigrees without a family history of prostate cancer. Family history included 1st and 2nd degree relatives. Controls had a screening prostate specific antigen (PSA) test <4 ng/ml at the time of ascertainment, had no personal history of prostate cancer, no record of a PSA test ≧4 ng/ml, and no record of abnormal digital rectal examination. The study included 683 unrelated, independent familial prostate cancer probands and 742 unrelated, independent control probands. Gleason score and tumor stage from surgical pathology was available for 96% of cases. The average age of diagnosis for cases was 60.3 years, and the average age at ascertainment screen for controls was 63.0 years.

### Finland

Samples (2,439) were recruited in Tampere and are all of Finnish origin. The mean age at diagnosis for these unselected consecutive prostate cancer patients was 68.7 years (range 43.1-94.9). The patients were diagnosed with the disease between 1993 and 2008 in the Tampere University Hospital, Department of Urology. Tampere University Hospital is a regional referral center in the area for all patients with prostate cancer, which results in an unselected, population-based collection of patients. The remainder of the cases, 248 men with family history of the disease not known to be related to each other, were recruited from all of Finland. Their mean age at diagnosis was 65.6 years (range 44-86.8). Study protocols were approved by the Ethics Committee of the Tampere University Hospital and the Ministry of Social Affairs and Health in Finland. All subjects gave written informed consent. For controls, 902 male samples and 903 female samples were used. Both of these Finnish population control groups consisted of DNA samples from anonymous, voluntary and healthy blood donors obtained from the Blood Center of the Finnish Red Cross in Tampere.

### Genotyping

Illumina genotyping. 1,968 and 35,382 Icelandic case- and control-samples respectively, were successfully assayed with the Infinium HumanHap300 SNP chip (Illumina, SanDiego, Calif., USA), containing 317,503 haplotype tagging SNPs derived from phase I of the International HapMap project. Of the SNPs assayed on the chip, 2,906 SNPs had a yield lower than 95%, 271 SNPs had a minor allele frequency, in the combined set of cases and controls, below 0.01 or were monomorphic. An additional 4,632 SNPs showed a significant distortion from Hardy-Weinberg equilibrium in the controls (P<1.0×10⁻³). In total, 6,983 unique SNPs were removed from the study. Thus, the analysis reported in the main text utilizes 310,520 SNPs. Any samples with a call rate below 98% were excluded from the analysis.

Replication genotyping. Single SNP genotyping of the SNPs reported in the main text for the four case-control groups from Iceland, The Netherlands, Spain and Chicago was carried out by deCODE genetics in Reykjavik, Iceland, applying the Centaurus (Nanogen) platform (Kutyavin, I. V. et al. Nucleic Acids Research 34, e128 (2006)). The quality of each Centaurus SNP assay was evaluated by genotyping each assay in the CEU and/or YRI HapMap samples and comparing the results with the HapMap publicly released data. Assays with >1.5% mismatch rate were not used and a linkage disequilibrium (LD) test was used for markers known to be in LD. We re-genotyped more than 10% of the samples and observed a mismatch rate lower than 0.5%. Genotyping of samples from Finland and Nashville was done using the same Centaurus assays as used in Iceland at the University of Tampere and Vanderbilt University, respectively, using standard protocols.

For each of the SNPs discussed in the main text, the yield was higher than 95% for those samples which genotyping was attempted for in every study group.

The SNP rs16902094 on 8q24 is not present on the Human Hap300 chip. Therefore, using a single SNP assay for genotyping, an attempt was made to genotype 6,900 and 800 individuals, respectively, of the 35,382 Icelandic controls as well as 1,860 Icelandic cases and all available individuals from the replication study groups.

Discovery of new SNP on 8q24 by Solexa re-sequencing. In order to search for new SNPs on 8q24, a 527 kb region (128113108-128640337 bp, Build 36) was sequenced using the Solexa re-sequencing platform (Illumina Inc.). From our set of about 2,000 cases; 800 were selected randomly and split into two DNA-pools, each with 400 samples. Similarly, 800 control individuals, not known to have prostate cancer, were randomly selected and split into two DNA-pools. Dilutions were prepared in duplicates and used for long-range PCR reactions (each amplimer consising of about 10 kb). PCR fragments were run on 0.8% agarose gels and the DNA visualized with BlueView (Sigma Inc.) and their sizes estimated with Hind III size marker (Fermentas Inc). Bands of correct sizes were excised out of the gels and purified with Qiagen gel extraction kit (Qiagen Inc.). The PCR products were quantified by picogreen assay (Invitrogen Inc.) as described by the manufacturer. The preparation of the Solexa DNA libraries, the cluster generation and DNA sequencing was done as described by Bentley et al (Bentley, D. R. et al. Nature 456, 53-9 (2008)). The SNP analysis pipeline is composed of four components: Alignment, SNP calling, Filtering and Association analysis. Promising SNPs were selected for further study/confirmation using Centaurus single track SNP assays.

### Statistical Analysis

Association analysis. For SNPs that were in strong LD, whenever the genotype of one SNP was missing for an individual, the genotype of the correlated SNP was used to provide partial information through a likelihood approach as previously described (Amundadottir, L. T. et al. Nat Genet 38, 652-8 (2006)). A likelihood procedure described in a previous publication Gretarsdottir, S. et al. Nat Genet 35, 131-8 (2003)) and implemented in the NEMO software was used for the association analyses.

We tested the association of an allele to prostate cancer using a standard likelihood ratio statistic that, if the subjects were unrelated, would have asymptotically a χ² distribution with one degree of freedom under the null hypothesis. Allelic frequencies rather than carrier frequencies are presented for the markers in the main text. Allele-specific ORs and associated P values were calculated assuming a multiplicative model for the two chromosomes of an individual (Falk, C. T. Rubinstein, P. Ann Hum Genet 51 (Pt 3), 227-33 (1987)). Results from multiple case-control groups were combined using a Mantel-Haenszel model (Mantel, N. & Haenszel, W. J Natl Cancer Inst. 22, 719-48 (1959)) in which the groups were allowed to have different population frequencies for alleles, haplotypes and genotypes but were assumed to have common relative risks (see Gudmundsson, J. et al. Nat Genet 39, 977-83 (2007) for a more detailed description of the association analysis).

The control groups from Iceland, The Netherlands, Spain, and Finland include both male and female controls. No significant difference between male and female controls was detected for SNPs presented in Table 1 for each of these four groups. Controls from other study groups include only males.

In order to for association for the SNP rs4962416 on 10q26, which is in the CEU section of the Hapmap database but absent from the Illumine Hap300 chip, we use a method based on haplotypes of two markers (rs7077275 and rs893856) present on the chip. We used a method we have previously employed, (Styrkarsdottir, U. et al. N Engl J Med 358, 2355-65 (2008)) that is an extension of the two-marker haplotype tagging method (Pe'er, I. et al. Nat Genet 38, 663-7 (2006)) and is similar in spirit to two other proposed methods (Nicolae, D. L. Genet Epidemiol 30, 718-27 (2006), Zaitlen, N., et al. Am J Hum Genet 80, 683-91 (2007)). We computed associations with a linear combination of the different haplotypes chosen to act as surrogates to HapMap markers in the regions. These calculations were based on 1,724 prostate cancer cases and 35,322 controls genotyped on chip.

Analysis of the CGEMS data. For the five individual study populations from the CGEMS study (Yeager, M. et al. Nat Genet 39, 645-9 (2007), Thomas, G. et al. Nat Genet 40, 310-5 (2008)) (ACS, ATBC, FPCC, HPFS, PLCO), when assessing the allelic effect we used the pre-computed data (released in spring, 2008) corresponding to “All case versus control (dichotomous), genotype trend effect model, adjusted”. When assessing the genotypic effect at each loci for the CGEMS study we used the pre-computed “All case versus control (dichotomous), genotype-specific effect model, adjusted, ALL (ACS, HPFS, FPCC, ATBC, PLCO)”.

Correction for relatedness. Some individuals in the Icelandic case-control groups were related to each other, causing the aforementioned χ² test statistic to have a mean >1. We estimated the inflation factor by using a previously described procedure (Stefansson, H. et al. Nat Genet 37, 129-37 (2005)) in which we simulated genotypes through the genealogy of the 37,350 Icelanders analyzed in the present study (number of simulations=100,000). The inflation factor was estimated to be 1.10. Results from the Icelandic samples presented in the main text are based on adjusting the X² statistics by dividing each of them by 1.10.

TABLE 1
Summary association results for the SNPs on 3q21.3, 8q24 and 19q13.2.

| Study | Cases | Controls | Frequency | | | |
|---|---|---|---|---|---|---|
| population | (N) | (N) | Cases | Controls | OR (95% CI) | P-value |

A. Results for rs10934853 [A] or rs4857841 [A] on 3q21.3

| | | | | | | |
|---|---|---|---|---|---|---|
| Iceland^{a} | 1,968 | 35,227 | 0.295 | 0.269 | 1.14 (1.06, 1.22) | 3.2E−04 |
| Chicago, Illinois | 1,077 | 1,003 | 0.313 | 0.273 | 1.21 (1.06, 1.39) | 4.4E−03 |
| Finland | 2,638 | 1,716 | 0.330 | 0.319 | 1.05 (0.96, 1.15) | 0.27 |
| The Netherlands | 1,084 | 1,827 | 0.306 | 0.286 | 1.10 (0.98, 1.24) | 0.10 |
| Nashville, | 596 | 687 | 0.283 | 0.270 | 1.07 (0.90, 1.27) | 0.47 |
| Tennessee | | | | | | |
| Spain | 811 | 1,605 | 0.306 | 0.314 | 0.96 (0.84, 1.09) | 0.54 |
| ACS^{b} | 1,758 | 1,775 | 0.300 | 0.258 | 1.25 (1.12, 1.39) | 4.3E−05 |
| ATBC^{b} | 928 | 921 | 0.309 | 0.319 | 0.96 (0.84, 1.10) | 0.59 |
| FPCC^{b} | 654 | 657 | 0.291 | 0.272 | 1.09 (0.92, 1.29) | 0.34 |
| HPFS^{b} | 595 | 609 | 0.313 | 0.278 | 1.18 (0.99, 1.40) | 0.070 |
| PLCO^{b} | 1,167 | 1,093 | 0.308 | 0.266 | 1.23 (1.08, 1.41) | 2.5E−03 |
| CAPS^{c} | 498 | 494 | 0.329 | 0.288 | 1.21 (1.00, 1.46) | 0.045 |
| All combined^{d} | 13,774 | 47,614 | — | 0.284 | 1.12 (1.08, 1.16) | 2.9E−10 |

B. Results for rs16902094 [G] or rs16902104 [T] on 8q24

| | | | | | | |
|---|---|---|---|---|---|---|
| Iceland^{a, e} | 1,858 | 6,853 | 0.168 | 0.136 | 1.28 (1.15, 1.41) | 3.5E−06 |
| Chicago, Illinois | 797 | 758 | 0.166 | 0.147 | 1.16 (0.95, 1.41) | 0.14 |
| Finland | 2,197 | 1,725 | 0.248 | 0.222 | 1.15 (1.03, 1.28) | 9.9E−03 |
| The Netherlands | 831 | 837 | 0.161 | 0.138 | 1.20 (0.99, 1.44) | 0.066 |
| Nashville, | 669 | 733 | 0.170 | 0.130 | 1.37 (1.11, 1.69) | 3.0E−03 |
| Tennessee | | | | | | |
| Spain | 643 | 952 | 0.162 | 0.137 | 1.21 (1.00, 1.48) | 0.055 |
| ACS^{b} | 1,759 | 1,774 | 0.156 | 0.132 | 1.22 (1.06, 1.39) | 4.3E−03 |
| ATBC^{b} | 929 | 920 | 0.255 | 0.193 | 1.43 (1.22, 1.67) | 1.0E−05 |
| FPCC^{b} | 656 | 657 | 0.152 | 0.145 | 1.06 (0.85, 1.31) | 0.61 |
| HPFS^{b} | 596 | 611 | 0.127 | 0.133 | 0.93 (0.74, 1.18) | 0.57 |
| PLCO^{b} | 1,167 | 1,093 | 0.145 | 0.137 | 1.09 (0.92, 1.30) | 0.31 |
| All combined^{d} | 12,102 | 16,913 | — | 0.150 | 1.21 (1.15, 1.26) | 6.2E−15 |

C. Results for rs445114 [T] on 8q24

| | | | | | | |
|---|---|---|---|---|---|---|
| Iceland^{a} | 1,727 | 35,382 | 0.710 | 0.672 | 1.20 (1.11, 1.29) | 5.0E−06 |
| The Netherlands | 910 | 1,832 | 0.676 | 0.650 | 1.13 (1.00, 1.27) | 0.048 |
| Spain | 490 | 1,387 | 0.660 | 0.624 | 1.17 (1.01, 1.36) | 0.041 |
| ACS | 1,757 | 1,768 | 0.651 | 0.618 | 1.15 (1.05, 1.27) | 4.0E−03 |
| ATBC | 925 | 919 | 0.702 | 0.661 | 1.22 (1.06, 1.40) | 6.6E−03 |
| FPCC | 655 | 655 | 0.647 | 0.635 | 1.05 (0.90, 1.24) | 0.52 |
| HPFS | 595 | 608 | 0.613 | 0.633 | 0.91 (0.77, 1.07) | 0.26 |
| PLCO | 1,175 | 1,100 | 0.641 | 0.618 | 1.13 (1.00, 1.28) | 5.7E−02 |
| All combined^{d} | 8,234 | 43,651 | — | 0.639 | 1.14 (1.10, 1.19) | 4.7E−10 |

D. Results for rs8102476 [C] on 19q13.2

| | | | | | | |
|---|---|---|---|---|---|---|
| Iceland^{a} | 1,941 | 35,330 | 0.517 | 0.495 | 1.09 (1.03, 1.17) | 6.4E−03 |
| Chicago, Illinois | 1,086 | 1,172 | 0.612 | 0.579 | 1.15 (1.02, 1.29) | 0.024 |
| Finland | 2,629 | 1,739 | 0.481 | 0.435 | 1.21 (1.11, 1.31) | 2.1E−05 |
| The Netherlands | 1,086 | 1,830 | 0.567 | 0.528 | 1.17 (1.05, 1.30) | 4.2E−03 |
| Nashville, Tennessee | 596 | 689 | 0.565 | 0.553 | 1.05 (0.90, 1.23) | 0.55 |
| Spain | 728 | 1,389 | 0.641 | 0.619 | 1.10 (0.96, 1.25) | 0.16 |
| ACS | 1,755 | 1,766 | 0.574 | 0.551 | 1.10 (1.00, 1.21) | 0.043 |
| ATBC | 926 | 919 | 0.473 | 0.461 | 1.05 (0.93, 1.20) | 0.43 |
| FPCC | 656 | 655 | 0.607 | 0.563 | 1.19 (1.02, 1.40) | 0.027 |
| HPFS | 595 | 609 | 0.574 | 0.57 | 1.03 (0.88, 1.21) | 0.74 |
| PLCO | 1,175 | 1,100 | 0.571 | 0.545 | 1.10 (0.98, 1.24) | 0.11 |
| All combined^{d} | 13,173 | 47,198 | — | 0.536 | 1.12 (1.08, 1.15) | 1.6E−11 |

All P values shown are two-sided. Shown are the corresponding numbers of cases and controls (N), allelic frequencies of variants in affected and control individuals, the allelic odds-ratio (OR) with 95% confidence interval (95% CI) and P value.
^{a}Results presented for Iceland were adjusted for relatedness (see Supplementary Methods).
^{b}The results for the five CGEMS groups on 3q21.3 and 8q24 are for the SNPs rs4857841[A] and rs16902104[T], which are highly correlated with rs10934853[A] and rs169020948[G], respectively (D′ and r² > 0.96 according to Icelandic and CEU HapMap data).
^{c}Results for the Swedish CAPS study group are for rs10934853[A] published by Duggan et al.⁸
^{d}For the combined study populations, the reported control frequency was the average, unweighted control frequency of the individual populations, while the OR and the P value were estimated using the Mantel-Haenszel model.
^{e}Results for rs16902104 [T] in Iceland: OR = 1.36; P-value 2.32E−10; based on imputation of 1,776 cases and 35,675 controls.

TABLE 2
Adjusted and unadjusted results for rs445114 and rs16902094 on 8q24.

| | | | rs445114 | | rs16902094 | |
|---|---|---|---|---|---|---|
| | | | Unadjusted | Adjusted | Unadjusted | Adjusted |
| | Cases | Controls | OR | OR | OR | OR |
| Study population | (n) | (n) | (P-value) | (P-value) | (P-value) | (P-value) |
| Iceland | 1607 | 6596 | 1.20 (4E−05) | 1.15 (4E−03) | 1.31 (1E−06) | 1.25 (1E−04) |
| Spain | 442 | 925 | 1.26 (0.007) | 1.21 (0.04) | 1.31 (0.02) | 1.21 (0.10) |
| The Netherlands | 743 | 837 | 1.09 (0.24) | 1.05 (0.52) | 1.23 (0.04) | 1.21 (0.07) |
| All Combined | 2792 | 8358 | 1.18 (1E−06) | 1.13 (8E−04) | 1.29 (8E−09) | 1.24 (5E−06) |

Shown are results for rs445114 before and after being adjusted for rs16902094 as well as results for rs16902094 before and after being adjusted for rs445114. The two SNPs are only correlated to a very small degree (D′ = 1 and r2 = 0.07 based on results from 5450 Icelanders). Results are only presented for individuals and populations where data is available for both SNPs.

TABLE 3
LD-information for rs16902094 and rs445114 on 8q24 and the previously published
cancer risk variants on 8q24.

| Marker-1 | Marker-2 (Comment) | D′ | r2 | Data set |
|---|---|---|---|---|
| rs16902094 | rs1447295 (Region 1 prostate cancer) | 0.03 | 3.2E−04 | deCODE generated CEU data |
| rs16902094 | rs16901979 (Region 2 prostate cancer) | 0.20 | 5.0E−03 | deCODE generated CEU data |
| rs16902094 | rs6983267 (Region 3 prostate- and colon cancer) | 0.14 | 4.8E−03 | deCODE generated CEU data |
| rs16902094 | rs13281615 (Breast cancer) | 0.61 | 0.063 | deCODE generated CEU data |
| rs16902094 | rs9642880 (Bladder cancer) | 0.06 | 5.1E−04 | deCODE generated CEU data |
| rs16902094 | rs13254738 (MEC-prostate cancer) | 0.43 | 0.070 | deCODE generated CEU data |
| rs16902094 | rs6983561 (MEC-prostate cancer) | 0.20 | 5.0E−03 | deCODE generated CEU data |
| rs16902094 | rs7000448 (MEC-prostate cancer) | 0.02 | 3.1E−05 | deCODE generated CEU data |
| rs16902094 | rs10090154 (MEC-prostate cancer) | 0.14 | 2.3E−04 | deCODE generated CEU data |
| rs445114 | rs1447295 (Region 1 prostate cancer) | 0.24 | 2.6E−03 | Public CEU-HapMap data |
| rs445114 | rs16901979 (Region 2 prostate cancer) | 0.27 | 2.8E−03 | Public CEU-HapMap data |
| rs445114 | rs6983267 (Region 3 prostate- and colon cancer) | 0.31 | 0.051 | Public CEU-HapMap data |
| rs445114 | rs13281615 (Breast cancer) | 0.76 | 0.44 | Public CEU-HapMap data |
| rs445114 | rs9642880 (Bladder cancer) | 0.11 | 6.3E−03 | Public CEU-HapMap data |
| rs445114 | rs10090154 (MEC-prostate cancer) | 0.11 | 5.3E−04 | Public CEU-HapMap data |
| rs445114 | rs13254738 (MEC-prostate cancer)¹ | 0.44 | 0.068 | Public CEU-HapMap data |
| rs445114 | rs6983561 (MEC-prostate cancer) | 0.27 | 2.8E−03 | Public CEU-HapMap data |
| rs445114 | rs7000448 (MEC-prostate cancer) | 0.60 | 0.13 | Public CEU-HapMap data |

Shown are the LD-characteristics of the two SNPs on 8q24 discussed in the main text and the various previously published cancer risk variants on 8q24 along with their original publication. No public CEU-HapMap results are available for rs16902094, hence, the data shown are based on in-house genotyping of the 90 CEPH Utah samples used in the HapMap project.

TABLE 4
Results for the Icelandic study population for the five prostate cancer
risk variants on 8q24.21 before and after being adjusted for each other.

| SNP | 8q24 | Control | Unadjusted | | Adjusted* | |
|---|---|---|---|---|---|---|
| [risk allele] | region | frequency | OR | P-value | OR | P-value |
| rs1447295[A] | Region-1 | 0.11 | 1.58 | 2.E−19 | 1.50 | 2.E−05 |
| rs16901979[A] | Region-2 | 0.04 | 1.80 | 2.E−14 | 1.63 | 2.E−10 |
| rs6983267[G] | Region-3 | 0.55 | 1.13 | 8.E−04 | 1.11 | 4.E−03 |
| rs445114[T] | Current | 0.67 | 1.20 | 6.E−06 | 1.17 | 1.E−04 |
| | finding | | | | | |
| rs16902094[G] | Current | 0.14 | 1.32 | 5.E−08 | 1.17 | 3.E−03 |
| | finding | | | | | |

The results shown are based on 1,793 cases and 35,465 controls from Iceland.
*The adjusted results for any one SNP is assessed jointly for the other four SNPs in the table.

TABLE 5
Model-free estimates of the genotype OR for markers on chr 3q21.3, 8q24 and 19q13.2.

| | 3q21.3 | | 8q24 | | 8q24 | | 19q13.2 | |
|---|---|---|---|---|---|---|---|---|
| | rs10934853[A] | | rs16902094[G] | | rs445114[T] | | rs8102476[C] | |
| | Genotypic OR | | Genotypic OR | | Genotypic OR | | Genotypic OR | |
| Study | Heterozygous | Homozygous | Heterozygous | Homozygous | Heterozygous | Homozygous | Heterozygous | Homozygous |
| population | carriers | carriers | carriers | carriers | carriers | carriers | carriers | carriers |
| Iceland | 1.18 | 1.25 | 1.24 | 1.80 | 1.29 | 1.49 | 1.07 | 1.20 |
| The | 1.09 | 1.22 | 1.23 | 1.29 | 1.31 | 1.38 | 1.17 | 1.37 |
| Netherlands | | | | | | | | |
| Spain | 1.02 | 1.04 | 1.26 | 1.22 | 1.13 | 1.33 | 1.14 | 1.39 |
| Chicago | 1.23 | 1.47 | 1.19 | 1.18 | NA | NA | 1.11 | 1.29 |
| Nashville | 1.13 | 1.04 | 1.32 | 2.09 | NA | NA | 1.15 | 1.13 |
| Finland | 1.05 | 1.11 | 1.14 | 1.34 | NA | NA | 1.22 | 1.44 |
| CGEMS^{a} | 1.17 | 1.29 | 1.18 | 1.37 | 1.08 | 1.26 | 1.12 | 1.21 |
| All | 1.11 | 1.21 | 1.22 | 1.47 | 1.20 | 1.37 | 1.14 | 1.29 |
| combined | | | | | | | | |

| | | | | |
|---|---|---|---|---|
| Full-versus | P-value = 0.67 | P-value = 0.81 | P-value = 0.25 | P-value = 0.91 |
| the multi- | | | | |
| plicative | | | | |
| model | | | | |

Shown are the genotypic ORs for heterozygous- and homozygous carriers of the risk alleles of the SNP discussed in the main text.
^{a}The results on 3q21.3-rs10934853 and 8q24-rs16902094 for the CGEMS groups are for the SNPs rs4857841[A] and rs16902104[T], respectively, which are highly correlated with rs10934853 and rs16902094 (D′ > 0.98 and r² > 0.96).
NA = not available.

TABLE 6
Association results in Iceland for variants reported to confer risk of prostate cancer.

| | | | | Frequency | | | |
|---|---|---|---|---|---|---|---|
| Marker, [risk allele] and (correlated marker(s))^{a} | Locus | Cases (N) | Controls (N) | Cases | Controls | OR (95% CI) | P-value |
| rs2710646 [A], (rs721048)⁵ | 2p15 | 1,882 | 35,145 | 0.224 | 0.203 | 1.14 (1.05, 1.24) | 2.5 × 10⁻³ |
| rs2660753 [T]³ | 3p12 | 1,725 | 35,362 | 0.110 | 0.100 | 1.11 (0.99, 1.25) | 0.075 |
| rs401681 [C]⁹ | 5p15 | 1,962 | 35,400 | 0.562 | 0.547 | 1.07 (1.00, 1.14) | 0.066 |
| rs9364554 [T]³ | 6q25 | 1,725 | 35,399 | 0.322 | 0.309 | 1.06 (0.99, 1.15) | 0.11 |
| rs10486567 [G]⁴ | 7p15 | 1,725 | 35,392 | 0.787 | 0.765 | 1.13 (1.04, 1.24) | 4.4 × 10⁻³ |
| rs6465657 [C]³ | 7q21 | 1,724 | 35,358 | 0.432 | 0.421 | 1.04 (0.97, 1.12) | 0.26 |
| rs1447295 [A]⁸ | 8q24 (1) | 1,821 | 35,470 | 0.165 | 0.111 | 1.58 (1.43, 1.74) | 2.2 × 10⁻¹⁹ |
| rs16901979 [A]¹ | 8q24 (2) | 1,726 | 35,403 | 0.073 | 0.042 | 1.80 (1.55, 2.09) | 2.5 × 10⁻¹⁴ |
| rs6983267 [G]⁶ | 8q24 (3) | 1,724 | 35,367 | 0.581 | 0.551 | 1.13 (1.05, 1.22) | 7.5 × 10⁻⁴ |
| rs1571801 [A]⁷ | 9q33^{b} | 1,721 | 35,303 | 0.261 | 0.276 | 0.93 (0.85, 1.01) | 0.068 |
| rs10993994 [T]^{3,4} | 10q11 | 1,727 | 35,397 | 0.410 | 0.384 | 1.11 (1.04, 1.20) | 3.7 × 10⁻³ |
| rs4962416 [C]⁴ | 10q26^{c} | 1,724 | 35,322 | 0.223 | 0.221 | 1.02 (0.94, 1.11) | 0.68 |
| rs10896450 [G], (rs10896449⁴, rs7931342³) | 11q13 | 1,951 | 35,394 | 0.501 | 0.469 | 1.13 (1.06, 1.21) | 2.5 × 10⁻⁴ |
| rs4430796 [A]² | 17q12 | 1,726 | 35,397 | 0.559 | 0.517 | 1.19 (1.10, 1.28) | 8.3 × 10⁻⁶ |
| rs11649743 [G]¹⁰ | 17q12 | 1,747 | 35,405 | 0.812 | 0.799 | 1.09 (0.99, 1.19) | 0.066 |
| rs1859962 [G]² | 17q24.3 | 1,746 | 35,124 | 0.493 | 0.455 | 1.16 (1.08, 1.25) | 3.7 × 10⁻⁵ |
| rs2735839 [G]³ | 19q13.33 | 1,726 | 35,376 | 0.879 | 0.865 | 1.14 (1.02, 1.27) | 0.021 |
| rs9623117 [C]¹¹ | 22q13^{b} | 1,724 | 35,389 | 0.208 | 0.208 | 1.00 (0.91, 1.10) | 0.99 |
| rs5945572 [A]⁵, (rs5945619³) | Xp11 | 1,899 | 35,384 | 0.416 | 0.369 | 1.22 (1.11, 1.34) | 6.1 × 10⁻⁵ |

^{a}Shown in the table are GWAS from Iceland for variants that have been identified through GWAS results (published up to February 2009) and the original publication(s). Highly correlated markers are shown in parenthesis as well as the study reporting them. All P values are two-sided. Shown are the corresponding numbers of cases and controls (N), allelic frequencies of variants in affected and control individuals, the allelic odds-ratio (OR) with 95% confidence interval (95% CI) and P value adjusted for relatedness.
^{b}The original results published for the loci on 9q33⁸ and 22q13¹⁹ were from a study on cases with aggressive prostate cancer. Results for these two loci in Icelandic cases (N = 693) with more aggressive prostate cancer (Gleason score >6 and/or T3 or higher and/or node positive and/or metastatic disease), using the same set of controls, were not significant (rs1571801; OR_{aggr} = 0.90 and P = 0.080, rs9623117; OR_{aggr} = 1.00 and P = 0.94).
^{c}The SNP marker, rs4962416, at the 10q26 locus is not on the Illumina Hap300 chip, results shown for it are based on a weighted combination of two marker haplotype generated from rs7077275 and rs893856 that are present on the chip and tag the SNP (rs4962416).
References:
¹Gudmundsson, J. et al. Genome-wide association study identifies a second prostate cancer susceptibility variant at 8q24. Nat Genet 39, 631-7 (2007).
²Gudmundsson, J. et al. Two variants on chromosome 17 confer prostate cancer risk, and the one in TCF2 protects against type 2 diabetes. Nat Genet 39, 977-83 (2007).
³Eeles, R. A. et al. Multiple newly identified loci associated with prostate cancer susceptibility. Nat Genet 40, 316-21 (2008).
⁴Thomas, G. et al. Multiple loci identified in a genome-wide association study of prostate cancer. Nat Genet 40, 310-5 (2008).
⁵Gudmundsson, J. et al. Common sequence variants on 2p15 and Xp11.22 confer susceptibility to prostate cancer. Nat Genet 40, 281-3 (2008).
⁶Yeager, M. et al. Genome-wide association study of prostate cancer identifies a second risk locus at 8q24. Nat Genet 39, 645-9 (2007).
⁷Duggan, D. et al. Two genome-wide association studies of aggressive prostate cancer implicate putative prostate tumor suppressor gene DAB2IP. J Natl Cancer Inst 99, 1836-44 (2007).
⁸Amundadottir, L. T. et al. A common variant associated with prostate cancer in European and African populations. Nat Genet 38, 652-8 (2006).
⁹Rafnar, T. et al. Sequence variants at the TERT-CLPTM1L locus associate with many cancer types. Nat Genet 41, 221-7 (2009).
¹⁰Sun, J. et al. Evidence for two independent prostate cancer risk-associated loci in the HNF1B gene at 17q12. Nat Genet 40, 1153-5 (2008).
¹¹Sun, J. et al. Sequence variants at 22q13 are associated with prostate cancer risk. Cancer Res 69, 10-5 (2009).

TABLE 7
Population distribution in Iceland of ORs for 22 prostate cancer
susceptibility variants.
Results from a multi-variant risk model analysis for prostate cancer in
Iceland based on susceptibility variants in tables 1 and 2. Results from
Iceland were used for all variants in table 1 and 2, except rs1571801 on
9q33 since its effect was in the opposite direction, and rs10896450 on
11q13 for which data for the refinement SNP in table 1 was used.
Odds ratios (OR) were calculated for all possible genotype combinations
based on 22 variants and expressed relative to the average general
population risk, assuming the multiplicative model between variants. The
combined OR estimates were then divided into OR-ranges and presented
along with the percentage of the population within each OR-range. The
general population risk was determined using a frequency-weighted
average risk for all possible genotypes.

| OR-range | Population percentage |
|---|---|
| <0.5 | 9.5% |
| 0.5-0.75 | 25.2% |
| 0.75-1 | 24.7% |
| 1-1.5 | 27.6% |
| 1.5-2 | 9.1% |
| 2-2.5 | 2.7% |
| >2.5 | 1.3% |

TABLE 8
Surrogate markers (based on HapMap CEU sample set; http://www.hapmap.org) on
Chromosome 3q21.3 with r² > 0.1 to rs10934853. Shown is; Surrogate marker name,
Anchor marker, the allele that is correlated with risk-allele of the anchor-marker, position of
surrogate marker in in NCBI Build 36, and D′, r2, and P-value of the correlation between
the markers. Allelic codes are A = 1, C = 2, G = 3, T = 4.

| | Anchor | | Pos in NCBI | | | | Seq |
|---|---|---|---|---|---|---|---|
| Marker | Marker | Allele | Build 36 | D′ | r2 | P-value | ID No: |
| rs4974416 | rs10934853 | 4 | 129060479 | 0.549015 | 0.104988 | 0.001394 | 5 |
| rs13095214 | rs10934853 | 2 | 129067204 | 0.340236 | 0.101148 | 0.002273 | 6 |
| rs11923862 | rs10934853 | 2 | 129067608 | 0.340236 | 0.101148 | 0.002273 | 7 |
| rs1543272 | rs10934853 | 4 | 129070913 | 0.340236 | 0.101148 | 0.002273 | 8 |
| rs6439086 | rs10934853 | 4 | 129072926 | 0.340236 | 0.101148 | 0.002273 | 9 |
| rs7644239 | rs10934853 | 4 | 129073582 | 0.340236 | 0.101148 | 0.002273 | 10 |
| rs7625264 | rs10934853 | 1 | 129074370 | 0.348986 | 0.109577 | 0.001701 | 11 |
| rs11921463 | rs10934853 | 3 | 129076449 | 0.340236 | 0.101148 | 0.002273 | 12 |
| rs13080277 | rs10934853 | 3 | 129076862 | 0.340236 | 0.101148 | 0.002273 | 13 |
| rs11926127 | rs10934853 | 3 | 129077438 | 0.340236 | 0.101148 | 0.002273 | 14 |
| rs7649674 | rs10934853 | 1 | 129078747 | 0.340236 | 0.101148 | 0.002273 | 15 |
| rs7616277 | rs10934853 | 1 | 129085350 | 0.340236 | 0.101148 | 0.002273 | 16 |
| rs6439094 | rs10934853 | 1 | 129111059 | 0.510683 | 0.103696 | 0.001284 | 17 |
| rs16838982 | rs10934853 | 2 | 129121762 | 0.430823 | 0.119625 | 0.000626 | 18 |
| rs2053016 | rs10934853 | 3 | 129127878 | 0.430823 | 0.119625 | 0.000626 | 19 |
| rs17203687 | rs10934853 | 4 | 129167438 | 0.629248 | 0.115111 | 0.000603 | 20 |
| rs16845806 | rs10934853 | 1 | 129193164 | 0.689141 | 0.241347 | 1.35E−06 | 21 |
| rs7630727 | rs10934853 | 2 | 129196138 | 0.773660 | 0.302088 | 6.54E−09 | 22 |
| rs1549876 | rs10934853 | 2 | 129197301 | 0.769894 | 0.289042 | 1.37E−08 | 23 |
| rs17282209 | rs10934853 | 2 | 129197886 | 1 | 0.318996 | 1.42E−09 | 24 |
| rs6439104 | rs10934853 | 2 | 129200392 | 0.912714 | 0.368255 | 9.89E−10 | 25 |
| rs1469659 | rs10934853 | 1 | 129203430 | 0.913656 | 0.368706 | 8.64E−10 | 26 |
| rs7611430 | rs10934853 | 3 | 129205905 | 0.760316 | 0.299946 | 2.38E−08 | 27 |
| rs6770337 | rs10934853 | 3 | 129207423 | 0.773660 | 0.302088 | 6.54E−09 | 28 |
| rs6777095 | rs10934853 | 1 | 129209327 | 1 | 0.213196 | 5.65E−06 | 29 |
| rs4602341 | rs10934853 | 1 | 129215781 | 1 | 0.409061 | 1.48E−11 | 30 |
| rs4857833 | rs10934853 | 3 | 129228387 | 0.836054 | 0.378151 | 9.54E−11 | 31 |
| rs6439108 | rs10934853 | 3 | 129228455 | 0.837551 | 0.387358 | 6.61E−11 | 32 |
| rs6764517 | rs10934853 | 1 | 129230531 | 1 | 0.302558 | 2.89E−08 | 33 |
| rs981447 | rs10934853 | 2 | 129236378 | 0.836054 | 0.378151 | 9.54E−11 | 34 |
| rs981446 | rs10934853 | 2 | 129236420 | 0.836054 | 0.378151 | 9.54E−11 | 35 |
| rs1469658 | rs10934853 | 1 | 129241904 | 1 | 0.441687 | 7.93E−13 | 36 |
| rs2335772 | rs10934853 | 2 | 129255226 | 0.840541 | 0.215326 | 2.03E−06 | 37 |
| rs1030656 | rs10934853 | 2 | 129256317 | 0.836054 | 0.378151 | 9.54E−11 | 38 |
| rs1030655 | rs10934853 | 1 | 129256366 | 0.830046 | 0.371256 | 2.89E−10 | 39 |
| rs2335771 | rs10934853 | 4 | 129262634 | 0.834997 | 0.374233 | 2.05E−10 | 40 |
| rs759945 | rs10934853 | 2 | 129262772 | 0.834088 | 0.385687 | 6.07E−09 | 41 |
| rs2075402 | rs10934853 | 3 | 129266952 | 0.836054 | 0.378151 | 9.54E−11 | 42 |
| rs1554534 | rs10934853 | 2 | 129282359 | 1 | 0.441687 | 7.93E−13 | 43 |
| rs3732402 | rs10934853 | 3 | 129288908 | 0.835070 | 0.387916 | 9.74E−11 | 44 |
| rs13091198 | rs10934853 | 4 | 129294727 | 0.778665 | 0.222860 | 0.000015 | 45 |
| rs11714052 | rs10934853 | 1 | 129297147 | 1 | 0.318996 | 1.42E−09 | 46 |
| rs6439113 | rs10934853 | 1 | 129299262 | 1 | 0.441687 | 7.93E−13 | 47 |
| rs6787614 | rs10934853 | 1 | 129300168 | 0.893981 | 0.282978 | 2.99E−07 | 48 |
| rs11720239 | rs10934853 | 3 | 129300645 | 1 | 0.318996 | 1.42E−09 | 49 |
| rs11715661 | rs10934853 | 4 | 129303536 | 1 | 0.318996 | 1.42E−09 | 50 |
| rs7641133 | rs10934853 | 4 | 129305010 | 1 | 0.873773 | 6.98E−26 | 51 |
| rs11924142 | rs10934853 | 2 | 129309308 | 1 | 0.441687 | 7.93E−13 | 52 |
| rs7650365 | rs10934853 | 3 | 129316693 | 0.851607 | 0.228552 | 2.97E−07 | 53 |
| rs6788879 | rs10934853 | 1 | 129318319 | 1 | 0.441687 | 7.93E−13 | 54 |
| rs6439115 | rs10934853 | 2 | 129318493 | 1 | 0.458763 | 4.98E−13 | 55 |
| rs4857836 | rs10934853 | 2 | 129318977 | 1 | 0.869707 | 4.67E−25 | 56 |
| rs4857837 | rs10934853 | 1 | 129319009 | 1 | 0.873773 | 6.98E−26 | 57 |
| rs11707462 | rs10934853 | 2 | 129321320 | 1 | 0.318996 | 1.42E−09 | 58 |
| rs9821568 | rs10934853 | 3 | 129321689 | 1 | 0.441687 | 7.93E−13 | 59 |
| rs6784159 | rs10934853 | 2 | 129326068 | 1 | 0.318996 | 1.42E−09 | 60 |
| rs2811475 | rs10934853 | 2 | 129328391 | 1 | 0.441687 | 7.93E−13 | 61 |
| rs13095660 | rs10934853 | 4 | 129330846 | 1 | 0.318996 | 1.42E−09 | 62 |
| rs6439116 | rs10934853 | 2 | 129331757 | 1 | 0.318996 | 1.42E−09 | 63 |
| rs6414310 | rs10934853 | 4 | 129339263 | 1 | 0.873773 | 6.98E−26 | 64 |
| rs2955102 | rs10934853 | 3 | 129348200 | 1 | 0.873773 | 6.98E−26 | 65 |
| rs11920225 | rs10934853 | 3 | 129354865 | 1 | 0.441687 | 7.93E−13 | 66 |
| rs11709066 | rs10934853 | 1 | 129357602 | 1 | 0.318996 | 1.42E−09 | 67 |
| rs11716941 | rs10934853 | 3 | 129358064 | 1 | 0.318996 | 1.42E−09 | 68 |
| rs2811472 | rs10934853 | 3 | 129361561 | 1 | 0.441687 | 7.93E−13 | 69 |
| rs13077913 | rs10934853 | 1 | 129365294 | 1 | 0.160178 | 0.000056 | 70 |
| rs13077790 | rs10934853 | 4 | 129365348 | 1 | 0.318996 | 1.42E−09 | 71 |
| rs2811473 | rs10934853 | 2 | 129367627 | 1 | 0.441687 | 7.93E−13 | 72 |
| rs2687728 | rs10934853 | 4 | 129367698 | 1 | 0.441687 | 7.93E−13 | 73 |
| rs10934850 | rs10934853 | 1 | 129369647 | 1 | 0.318996 | 1.42E−09 | 74 |
| rs872267 | rs10934853 | 4 | 129370757 | 1 | 0.873773 | 6.98E−26 | 75 |
| rs2687731 | rs10934853 | 4 | 129371384 | 1 | 0.442786 | 2.01E−12 | 76 |
| rs3122174 | rs10934853 | 3 | 129372065 | 1 | 0.458763 | 4.28E−13 | 77 |
| rs2999051 | rs10934853 | 2 | 129372094 | 0.943681 | 0.496406 | 5.84E−14 | 78 |
| rs13067650 | rs10934853 | 1 | 129372232 | 1 | 0.318996 | 1.58E−09 | 79 |
| rs2248668 | rs10934853 | 2 | 129373439 | 0.916396 | 0.384339 | 4.99E−10 | 80 |
| rs2955121 | rs10934853 | 3 | 129374086 | 1 | 0.441687 | 7.93E−13 | 81 |
| rs11706455 | rs10934853 | 1 | 129374681 | 1 | 0.311800 | 5.35E−09 | 82 |
| rs2999052 | rs10934853 | 2 | 129374727 | 1 | 0.873773 | 6.98E−26 | 83 |
| rs11715394 | rs10934853 | 4 | 129376254 | 1 | 0.318996 | 1.42E−09 | 84 |
| rs2687729 | rs10934853 | 2 | 129377916 | 1 | 0.873773 | 6.98E−26 | 85 |
| rs2811478 | rs10934853 | 4 | 129382314 | 1 | 0.162113 | 0.000023 | 86 |
| rs2999060 | rs10934853 | 2 | 129383184 | 1 | 0.441687 | 7.93E−13 | 87 |
| rs2999056 | rs10934853 | 1 | 129386212 | 1 | 0.407825 | 3.02E−11 | 88 |
| rs2955123 | rs10934853 | 2 | 129386368 | 1 | 0.477212 | 2.20E−13 | 89 |
| rs2811517 | rs10934853 | 3 | 129386880 | 1 | 0.425837 | 8.26E−12 | 90 |
| rs2811516 | rs10934853 | 2 | 129388141 | 0.895338 | 0.702716 | 2.16E−16 | 91 |
| rs2811515 | rs10934853 | 4 | 129388208 | 1 | 0.873773 | 6.98E−26 | 92 |
| rs2811514 | rs10934853 | 2 | 129390619 | 1 | 0.869707 | 6.10E−25 | 93 |
| rs2811512 | rs10934853 | 4 | 129394510 | 1 | 0.873773 | 6.98E−26 | 94 |
| rs2811511 | rs10934853 | 4 | 129395678 | 1 | 0.300399 | 5.76E−09 | 95 |
| rs883238 | rs10934853 | 3 | 129395953 | 1 | 0.873773 | 6.98E−26 | 96 |
| rs940061 | rs10934853 | 3 | 129396404 | 1 | 0.441687 | 7.93E−13 | 97 |
| rs2811510 | rs10934853 | 2 | 129397202 | 1 | 0.441687 | 7.93E−13 | 98 |
| rs2811483 | rs10934853 | 2 | 129397363 | 1 | 0.441687 | 7.93E−13 | 99 |
| rs2811484 | rs10934853 | 1 | 129397543 | 1 | 0.441687 | 7.93E−13 | 100 |
| rs2687730 | rs10934853 | 4 | 129398147 | 1 | 0.318996 | 1.58E−09 | 101 |
| rs2811509 | rs10934853 | 4 | 129399241 | 1 | 0.873773 | 9.12E−26 | 102 |
| rs2492285 | rs10934853 | 1 | 129400690 | 1 | 0.873773 | 6.98E−26 | 103 |
| rs2687720 | rs10934853 | 1 | 129401645 | 1 | 0.873773 | 9.12E−26 | 104 |
| rs2811508 | rs10934853 | 3 | 129402713 | 1 | 0.873773 | 6.98E−26 | 105 |
| rs2811486 | rs10934853 | 3 | 129402765 | 1 | 0.318996 | 1.42E−09 | 106 |
| rs6439119 | rs10934853 | 4 | 129405877 | 1 | 0.865370 | 1.08E−24 | 107 |
| rs2955125 | rs10934853 | 3 | 129407882 | 1 | 0.441687 | 7.93E−13 | 108 |
| rs2955126 | rs10934853 | 1 | 129408944 | 1 | 0.300399 | 6.38E−09 | 109 |
| rs2955127 | rs10934853 | 3 | 129409206 | 1 | 0.441687 | 7.93E−13 | 110 |
| rs4293718 | rs10934853 | 3 | 129411810 | 0.878205 | 0.119753 | 0.000084 | 111 |
| rs2955129 | rs10934853 | 4 | 129411897 | 1 | 0.318996 | 1.42E−09 | 112 |
| rs7374072 | rs10934853 | 2 | 129413556 | 1 | 0.910646 | 2.53E−26 | 113 |
| rs2999090 | rs10934853 | 2 | 129414030 | 1 | 0.318996 | 1.42E−09 | 114 |
| rs7372439 | rs10934853 | 1 | 129414092 | 1 | 0.873773 | 6.98E−26 | 115 |
| rs4857871 | rs10934853 | 2 | 129416476 | 1 | 0.873773 | 6.98E−26 | 116 |
| rs4857872 | rs10934853 | 4 | 129416512 | 1 | 0.826087 | 1.31E−19 | 117 |
| rs4857873 | rs10934853 | 1 | 129416908 | 1 | 0.873773 | 6.98E−26 | 118 |
| rs6770140 | rs10934853 | 4 | 129417019 | 1 | 0.873773 | 6.98E−26 | 119 |
| rs4384971 | rs10934853 | 2 | 129417464 | 1 | 0.873773 | 6.98E−26 | 120 |
| rs2999089 | rs10934853 | 3 | 129417849 | 1 | 0.300399 | 5.76E−09 | 121 |
| rs6439121 | rs10934853 | 3 | 129418151 | 0.951760 | 0.830101 | 1.99E−22 | 122 |
| rs2254379 | rs10934853 | 2 | 129420016 | 1 | 0.441687 | 7.93E−13 | 123 |
| rs2955130 | rs10934853 | 3 | 129420504 | 1 | 0.318996 | 1.42E−09 | 124 |
| rs9814834 | rs10934853 | 3 | 129420827 | 1 | 0.873773 | 6.98E−26 | 125 |
| rs2955132 | rs10934853 | 2 | 129422916 | 1 | 0.441687 | 7.93E−13 | 126 |
| rs9845651 | rs10934853 | 4 | 129423043 | 1 | 0.873773 | 6.98E−26 | 127 |
| rs6439122 | rs10934853 | 1 | 129423224 | 1 | 0.873773 | 6.98E−26 | 128 |
| rs9873786 | rs10934853 | 1 | 129425069 | 1 | 0.873773 | 6.98E−26 | 129 |
| rs4857838 | rs10934853 | 1 | 129426018 | 1 | 0.873773 | 6.98E−26 | 130 |
| rs6775988 | rs10934853 | 2 | 129427167 | 1 | 0.873773 | 6.98E−26 | 131 |
| rs9830294 | rs10934853 | 3 | 129429478 | 1 | 0.873773 | 6.98E−26 | 132 |
| rs4857877 | rs10934853 | 1 | 129430750 | 1 | 0.865370 | 1.08E−24 | 133 |
| rs2999086 | rs10934853 | 3 | 129433938 | 1 | 0.318996 | 1.42E−09 | 134 |
| rs2999085 | rs10934853 | 2 | 129434249 | 1 | 0.280757 | 2.62E−08 | 135 |
| rs2999084 | rs10934853 | 4 | 129435178 | 1 | 0.300399 | 5.76E−09 | 136 |
| rs2999083 | rs10934853 | 3 | 129437124 | 1 | 0.318996 | 1.42E−09 | 137 |
| rs2999081 | rs10934853 | 4 | 129438962 | 1 | 0.270517 | 3.37E−08 | 138 |
| rs2999079 | rs10934853 | 1 | 129439922 | 1 | 0.270517 | 7.10E−08 | 139 |
| rs4074440 | rs10934853 | 2 | 129440703 | 1 | 0.873773 | 9.12E−26 | 140 |
| rs2955077 | rs10934853 | 1 | 129441559 | 1 | 0.135053 | 0.000277 | 141 |
| rs9843281 | rs10934853 | 1 | 129444086 | 1 | 0.873773 | 9.12E−26 | 142 |
| rs2999073 | rs10934853 | 2 | 129445019 | 1 | 0.300399 | 6.38E−09 | 143 |
| rs2955085 | rs10934853 | 3 | 129445447 | 1 | 0.318996 | 1.42E−09 | 144 |
| rs2999072 | rs10934853 | 2 | 129445566 | 1 | 0.318996 | 1.42E−09 | 145 |
| rs13434079 | rs10934853 | 1 | 129446138 | 1 | 0.873773 | 6.98E−26 | 146 |
| rs2955088 | rs10934853 | 2 | 129446400 | 1 | 0.318996 | 1.42E−09 | 147 |
| rs2999070 | rs10934853 | 2 | 129447341 | 1 | 0.318996 | 1.42E−09 | 148 |
| rs17343355 | rs10934853 | 2 | 129449481 | 0.840753 | 0.208333 | 1.84E−06 | 149 |
| rs2955090 | rs10934853 | 4 | 129452061 | 1 | 0.318996 | 1.42E−09 | 150 |
| rs2955091 | rs10934853 | 3 | 129454051 | 1 | 0.318996 | 1.42E−09 | 151 |
| rs2999069 | rs10934853 | 3 | 129454299 | 1 | 0.318996 | 1.42E−09 | 152 |
| rs2955092 | rs10934853 | 2 | 129455218 | 1 | 0.318996 | 1.58E−09 | 153 |
| rs2955094 | rs10934853 | 1 | 129459613 | 1 | 0.318996 | 1.42E−09 | 154 |
| rs2955095 | rs10934853 | 1 | 129460355 | 1 | 0.318996 | 1.42E−09 | 155 |
| rs2955096 | rs10934853 | 1 | 129460556 | 1 | 0.318996 | 1.42E−09 | 156 |
| rs2999068 | rs10934853 | 1 | 129460668 | 1 | 0.255935 | 1.34E−07 | 157 |
| rs2999067 | rs10934853 | 3 | 129461221 | 1 | 0.255935 | 1.22E−07 | 158 |
| rs2955099 | rs10934853 | 3 | 129462344 | 1 | 0.318996 | 1.42E−09 | 159 |
| rs2999066 | rs10934853 | 1 | 129462976 | 1 | 0.318996 | 1.42E−09 | 160 |
| rs2999065 | rs10934853 | 4 | 129464128 | 1 | 0.300399 | 5.76E−09 | 161 |
| rs2811545 | rs10934853 | 3 | 129465838 | 1 | 0.291803 | 2.21E−08 | 162 |
| rs2999035 | rs10934853 | 4 | 129465899 | 1 | 0.407825 | 4.54E−11 | 163 |
| rs2811544 | rs10934853 | 1 | 129466185 | 1 | 0.318996 | 1.42E−09 | 164 |
| rs2811543 | rs10934853 | 1 | 129466486 | 1 | 0.300399 | 6.38E−09 | 165 |
| rs2811541 | rs10934853 | 3 | 129467484 | 1 | 0.300399 | 9.60E−09 | 166 |
| rs2811540 | rs10934853 | 1 | 129468259 | 1 | 0.246449 | 1.68E−07 | 167 |
| rs2811539 | rs10934853 | 3 | 129469163 | 1 | 0.289731 | 8.29E−09 | 168 |
| rs2811538 | rs10934853 | 4 | 129469321 | 1 | 0.318996 | 1.42E−09 | 169 |
| rs2811396 | rs10934853 | 1 | 129470046 | 1 | 0.300399 | 7.83E−09 | 170 |
| rs2811400 | rs10934853 | 3 | 129470557 | 1 | 0.317266 | 4.24E−09 | 171 |
| rs2811537 | rs10934853 | 4 | 129470614 | 1 | 0.318996 | 1.42E−09 | 172 |
| rs2999064 | rs10934853 | 2 | 129470930 | 0.838076 | 0.387783 | 4.45E−09 | 173 |
| rs2811536 | rs10934853 | 4 | 129471609 | 1 | 0.280757 | 3.18E−08 | 174 |
| rs2811534 | rs10934853 | 4 | 129473798 | 1 | 0.289731 | 9.15E−09 | 175 |
| rs2811413 | rs10934853 | 2 | 129473839 | 0.867809 | 0.721362 | 9.63E−20 | 176 |
| rs2811415 | rs10934853 | 1 | 129474217 | 0.709631 | 0.208846 | 0.000010 | 177 |
| rs2811533 | rs10934853 | 2 | 129474419 | 1 | 0.318996 | 1.42E−09 | 178 |
| rs2811416 | rs10934853 | 4 | 129474628 | 1 | 0.336138 | 7.43E−10 | 179 |
| rs2811532 | rs10934853 | 2 | 129474860 | 1 | 0.300399 | 7.83E−09 | 180 |
| rs2811531 | rs10934853 | 2 | 129475185 | 1 | 0.280757 | 3.18E−08 | 181 |
| rs2955100 | rs10934853 | 3 | 129476015 | 1 | 0.318996 | 2.72E−09 | 182 |
| rs2999061 | rs10934853 | 4 | 129476313 | 1 | 0.318996 | 1.42E−09 | 183 |
| rs2811529 | rs10934853 | 2 | 129476850 | 1 | 0.873773 | 6.98E−26 | 184 |
| rs2811527 | rs10934853 | 4 | 129477294 | 1 | 0.318996 | 1.42E−09 | 185 |
| rs2811373 | rs10934853 | 4 | 129480119 | 1 | 0.318996 | 1.42E−09 | 186 |
| rs2811525 | rs10934853 | 1 | 129482120 | 1 | 0.318996 | 1.42E−09 | 187 |
| rs7374952 | rs10934853 | 4 | 129484057 | 1 | 0.300399 | 6.38E−09 | 188 |
| rs7374227 | rs10934853 | 3 | 129484205 | 1 | 0.318996 | 1.42E−09 | 189 |
| rs4593050 | rs10934853 | 4 | 129487221 | 1 | 0.318996 | 1.42E−09 | 190 |
| rs6439124 | rs10934853 | 1 | 129490156 | 1 | 0.318996 | 1.42E−09 | 191 |
| rs7373998 | rs10934853 | 1 | 129490913 | 1 | 0.318996 | 1.42E−09 | 192 |
| rs2955101 | rs10934853 | 1 | 129492302 | 1 | 0.318996 | 1.42E−09 | 193 |
| rs2811519 | rs10934853 | 1 | 129495566 | 1 | 0.300399 | 5.76E−09 | 194 |
| rs2811518 | rs10934853 | 2 | 129496335 | 1 | 0.318996 | 1.42E−09 | 195 |
| rs2955103 | rs10934853 | 2 | 129497926 | 0.891861 | 0.445697 | 7.84E−13 | 196 |
| rs2811388 | rs10934853 | 1 | 129501137 | 1 | 0.318996 | 1.42E−09 | 197 |
| rs2999036 | rs10934853 | 3 | 129503391 | 1 | 0.318996 | 1.42E−09 | 198 |
| rs2811390 | rs10934853 | 4 | 129504171 | 1 | 0.318996 | 1.42E−09 | 199 |
| rs2811391 | rs10934853 | 1 | 129505058 | 1 | 0.318996 | 1.58E−09 | 200 |
| rs2811393 | rs10934853 | 4 | 129506689 | 1 | 0.311883 | 4.81E−09 | 201 |
| rs2037965 | rs10934853 | 3 | 129507734 | 1 | 0.318996 | 1.42E−09 | 202 |
| rs2811397 | rs10934853 | 2 | 129509927 | 1 | 0.361902 | 2.73E−10 | 203 |
| rs6805582 | rs10934853 | 1 | 129511698 | 1 | 0.318996 | 1.42E−09 | 204 |
| rs6805621 | rs10934853 | 4 | 129511894 | 1 | 0.318996 | 1.76E−09 | 205 |
| rs6794591 | rs10934853 | 4 | 129513909 | 1 | 0.318996 | 1.76E−09 | 206 |
| rs16843876 | rs10934853 | 3 | 129515230 | 1 | 0.318996 | 1.42E−09 | 207 |
| rs11706852 | rs10934853 | 1 | 129515577 | 1 | 0.318996 | 1.42E−09 | 208 |
| rs11706826 | rs10934853 | 4 | 129515681 | 1 | 0.318996 | 1.42E−09 | 209 |
| rs11706908 | rs10934853 | 1 | 129515738 | 1 | 0.318996 | 1.42E−09 | 210 |
| rs6771646 | rs10934853 | 3 | 129517225 | 1 | 0.318996 | 1.42E−09 | 211 |
| rs13095166 | rs10934853 | 4 | 129519476 | 1 | 0.540083 | 1.62E−15 | 212 |
| rs10934853 | rs10934853 | 1 | 129521063 | 1 | 1 | 0 | 213 |
| rs12486127 | rs10934853 | 1 | 129521379 | 1 | 0.571734 | 3.59E−15 | 214 |
| rs12486156 | rs10934853 | 4 | 129521524 | 1 | 0.526627 | 6.46E−15 | 215 |
| rs11708733 | rs10934853 | 1 | 129522585 | 1 | 0.129173 | 0.000144 | 216 |
| rs6772407 | rs10934853 | 4 | 129527062 | 1 | 0.318996 | 1.42E−09 | 217 |
| rs4857841 | rs10934853 | 1 | 129529333 | 1 | 1 | 1.14E−31 | 218 |
| rs11710704 | rs10934853 | 1 | 129529926 | 1 | 0.318996 | 1.42E−09 | 219 |
| rs16844002 | rs10934853 | 4 | 129536177 | 1 | 0.318996 | 1.42E−09 | 220 |
| rs6798749 | rs10934853 | 1 | 129539587 | 1 | 0.318996 | 1.42E−09 | 221 |
| rs1735558 | rs10934853 | 4 | 129542300 | 1 | 0.506641 | 1.36E−14 | 222 |
| rs4857879 | rs10934853 | 2 | 129546808 | 1 | 0.540083 | 1.62E−15 | 223 |
| rs11721213 | rs10934853 | 4 | 129550131 | 1 | 0.318996 | 1.42E−09 | 224 |
| rs1735549 | rs10934853 | 1 | 129554499 | 1 | 0.743448 | 7.86E−21 | 225 |
| rs1735546 | rs10934853 | 2 | 129558088 | 1 | 0.780702 | 8.12E−22 | 226 |
| rs12632366 | rs10934853 | 3 | 129560248 | 0.910338 | 0.213353 | 6.32E−07 | 227 |
| rs1735545 | rs10934853 | 3 | 129563950 | 1 | 0.755518 | 7.24E−22 | 228 |
| rs1702122 | rs10934853 | 2 | 129566022 | 0.945706 | 0.675705 | 1.17E−17 | 229 |
| rs1108313 | rs10934853 | 2 | 129567780 | 0.912756 | 0.221910 | 3.58E−07 | 230 |
| rs1735538 | rs10934853 | 2 | 129574792 | 0.899287 | 0.674071 | 2.83E−17 | 231 |
| rs1702119 | rs10934853 | 3 | 129577183 | 0.947283 | 0.712980 | 1.03E−18 | 232 |
| rs1702118 | rs10934853 | 2 | 129577968 | 1 | 0.379184 | 4.19E−11 | 233 |
| rs3021461 | rs10934853 | 3 | 129578342 | 1 | 0.717742 | 1.10E−20 | 234 |
| rs2977565 | rs10934853 | 4 | 129578457 | 1 | 0.717742 | 1.10E−20 | 235 |
| rs2293947 | rs10934853 | 3 | 129580186 | 1 | 0.260997 | 4.66E−08 | 236 |
| rs741925 | rs10934853 | 4 | 129592606 | 1 | 0.379184 | 3.69E−11 | 237 |
| rs729847 | rs10934853 | 4 | 129593460 | 0.759508 | 0.517548 | 5.79E−13 | 238 |
| rs1702134 | rs10934853 | 4 | 129593891 | 0.910626 | 0.381317 | 1.03E−08 | 239 |
| rs1620440 | rs10934853 | 2 | 129594997 | 1 | 0.379184 | 3.69E−11 | 240 |
| rs7632169 | rs10934853 | 2 | 129597277 | 0.734951 | 0.533499 | 1.93E−13 | 241 |
| rs1735527 | rs10934853 | 2 | 129598071 | 1 | 0.361902 | 1.89E−10 | 242 |
| rs760383 | rs10934853 | 2 | 129602255 | 0.757203 | 0.500983 | 4.52E−13 | 243 |
| rs11705709 | rs10934853 | 3 | 129602564 | 0.623665 | 0.161261 | 0.000073 | 244 |
| rs11705891 | rs10934853 | 3 | 129603045 | 0.633150 | 0.164416 | 0.000056 | 245 |
| rs2999031 | rs10934853 | 4 | 129604192 | 0.901429 | 0.179119 | 3.69E−06 | 246 |
| rs6780368 | rs10934853 | 4 | 129604729 | 1 | 0.330855 | 1.06E−09 | 247 |
| rs2659685 | rs10934853 | 1 | 129605086 | 0.773981 | 0.573281 | 3.63E−15 | 248 |
| rs11715947 | rs10934853 | 4 | 129605270 | 0.633150 | 0.164416 | 0.000056 | 249 |
| rs1735537 | rs10934853 | 3 | 129605510 | 0.728411 | 0.507762 | 2.20E−13 | 250 |
| rs11717030 | rs10934853 | 1 | 129605978 | 0.633150 | 0.164416 | 0.000056 | 251 |
| rs2977564 | rs10934853 | 2 | 129606476 | 1 | 0.201656 | 4.61E−09 | 252 |
| rs2939820 | rs10934853 | 3 | 129610333 | 0.811599 | 0.128124 | 0.000053 | 253 |
| rs3828417 | rs10934853 | 2 | 129610944 | 0.798189 | 0.113308 | 0.001103 | 254 |
| rs4527399 | rs10934853 | 2 | 129620819 | 1 | 0.106079 | 0.000632 | 255 |
| rs4521245 | rs10934853 | 3 | 129620888 | 1 | 0.124825 | 0.000166 | 256 |
| rs1806462 | rs10934853 | 3 | 129689308 | 0.638929 | 0.104036 | 0.000908 | 257 |
| rs2860228 | rs10934853 | 3 | 129692357 | 0.819443 | 0.165843 | 0.000019 | 258 |
| rs9851497 | rs10934853 | 4 | 129695216 | 0.410144 | 0.108815 | 0.001271 | 259 |
| rs6789646 | rs10934853 | 4 | 129698465 | 0.658655 | 0.107934 | 0.000395 | 260 |
| rs7629791 | rs10934853 | 3 | 129701100 | 0.590491 | 0.129826 | 0.000185 | 261 |
| rs2713576 | rs10934853 | 2 | 129705990 | 0.658655 | 0.107934 | 0.000395 | 262 |
| rs2659698 | rs10934853 | 2 | 129709054 | 0.410144 | 0.108815 | 0.001271 | 263 |

TABLE 9
Surrogate markers (based on HapMap CEU sample set; http://www.hapmap.org) on
Chromosome 8q24.21 with r² > 0.1 to rs16902094. Shown is; Surrogate marker
name, Anchor marker, the allele that is correlated with risk-allele of the anchor-marker,
position of the surrogate marker in NCBI Build 36, D′, r², and P-value of the
correlation between the markers. Allelic codes are A = 1, C = 2, G = 3, T = 4.

| | Anchor | | Pos in NCBI | | | | Seq |
|---|---|---|---|---|---|---|---|
| Marker | Marker | Allele | Build 36 | D′ | r2 | P-valuE | ID No: |
| rs1840709 | rs16902094 | 2 | 128168637 | 0.663762 | 0.152714 | 0.000102 | 264 |
| rs3857883 | rs16902094 | 2 | 128169788 | 0.721346 | 0.144433 | 0.000112 | 265 |
| rs1456316 | rs16902094 | 4 | 128170030 | 0.722980 | 0.146763 | 0.000097 | 266 |
| rs1456315 | rs16902094 | 1 | 128173119 | 0.620969 | 0.124235 | 0.000718 | 267 |
| rs7006409 | rs16902094 | 3 | 128180611 | 0.529036 | 0.106428 | 0.004138 | 268 |
| rs4871775 | rs16902094 | 2 | 128340277 | 0.359594 | 0.102769 | 0.003347 | 269 |
| rs4871779 | rs16902094 | 3 | 128348189 | 0.437239 | 0.110395 | 0.001125 | 270 |
| rs13251915 | rs16902094 | 4 | 128377137 | 0.646468 | 0.221140 | 3.49E−06 | 271 |
| rs283720 | rs16902094 | 1 | 128379147 | 0.706547 | 0.233649 | 1.18E−06 | 272 |
| rs283704 | rs16902094 | 2 | 128384764 | 1.000.000 | 0.117647 | 5.15E−06 | 273 |
| rs283705 | rs16902094 | 4 | 128386632 | 0.861315 | 0.100497 | 0.000306 | 274 |
| rs16902094 | rs16902094 | 3 | 128389528 | 1 | 1 | | 275 |
| rs453875 | rs16902094 | 2 | 128390593 | 0.883915 | 0.132375 | 0.000022 | 276 |
| 5G0851738 | rs16902094 | 2 | 128390595 | 1 | 0.942761 | 6.70E−35 | 277 |
| rs11785664 | rs16902094 | 4 | 128399606 | 1.000.000 | 0.109375 | 9.75E−06 | 278 |
| rs622556 | rs16902094 | 3 | 128402379 | 1.000.000 | 0.150442 | 4.71E−07 | 279 |
| rs452529 | rs16902094 | 3 | 128402441 | 1.000.000 | 0.150442 | 4.71E−07 | 280 |
| rs400818 | rs16902094 | 3 | 128405728 | 1.000.000 | 0.155689 | 3.27E−07 | 281 |
| rs386883 | rs16902094 | 4 | 128406053 | 1.000.000 | 0.176517 | 1.27E−07 | 282 |
| rs377649 | rs16902094 | 1 | 128406423 | 1.000.000 | 0.155689 | 3.27E−07 | 283 |
| rs432470 | rs16902094 | 1 | 128408226 | 1.000.000 | 0.121951 | 3.71E−06 | 284 |
| rs424281 | rs16902094 | 4 | 128408608 | 1.000.000 | 0.121951 | 3.71E−06 | 285 |
| rs16902103 | rs16902094 | 2 | 128409556 | 0.938176 | 0.784640 | 1.19E−18 | 286 |
| rs16902104 | rs16902094 | 4 | 128410090 | 0.938176 | 0.784640 | 1.19E−18 | 287 |
| rs1668875 | rs16902094 | 2 | 128410285 | 0.884726 | 0.123979 | 0.000022 | 288 |
| rs7002712 | rs16902094 | 1 | 128410794 | 1.000.000 | 0.113456 | 7.10E−06 | 289 |
| rs587948 | rs16902094 | 1 | 128410862 | 0.869693 | 0.115901 | 0.000122 | 290 |
| rs623401 | rs16902094 | 3 | 128410909 | 0.882468 | 0.119243 | 0.000031 | 291 |
| rs16902118 | rs16902094 | 3 | 128417799 | 0.755579 | 0.539596 | 1.61E−12 | 292 |
| rs10095860 | rs16902094 | 2 | 128423967 | 0.755926 | 0.210795 | 3.49E−06 | 293 |
| rs16902121 | rs16902094 | 1 | 128424100 | 0.692044 | 0.452952 | 2.01E−10 | 294 |
| rs13256275 | rs16902094 | 1 | 128425408 | 0.856377 | 0.100507 | 0.000668 | 295 |
| rs11785277 | rs16902094 | 2 | 128434265 | 0.692044 | 0.452952 | 2.01E−10 | 296 |
| rs11774827 | rs16902094 | 2 | 128434523 | 0.696446 | 0.485008 | 3.83E−11 | 297 |
| rs11782693 | rs16902094 | 3 | 128435626 | 0.692044 | 0.452952 | 2.01E−10 | 298 |
| rs11782700 | rs16902094 | 4 | 128435678 | 0.676666 | 0.440592 | 1.15E−08 | 299 |
| rs11782735 | rs16902094 | 4 | 128435786 | 0.692044 | 0.452952 | 2.01E−10 | 300 |
| rs11783559 | rs16902094 | 4 | 128436107 | 0.692044 | 0.452952 | 2.01E−10 | 301 |
| rs11783615 | rs16902094 | 1 | 128436189 | 0.692044 | 0.452952 | 2.01E−10 | 302 |
| rs11784125 | rs16902094 | 3 | 128449102 | 0.692044 | 0.452952 | 2.01E−10 | 303 |
| rs11776260 | rs16902094 | 3 | 128451670 | 0.679839 | 0.452622 | 1.19E−09 | 304 |
| rs11774907 | rs16902094 | 2 | 128453272 | 0.635583 | 0.403965 | 1.53E−09 | 305 |
| rs16902127 | rs16902094 | 1 | 128453599 | 0.649091 | 0.410019 | 5.11E−09 | 306 |
| rs7015780 | rs16902094 | 2 | 128458689 | 1.000.000 | 0.105398 | 0.000013 | 307 |
| rs731900 | rs16902094 | 4 | 128459842 | 0.590039 | 0.230440 | 4.35E−06 | 308 |

TABLE 10
Surrogate markers (based on HapMap CEU sample set; http://www.hapmap.org) on
Chromosome 8q24.21 with r² > 0.1 to rs445114. Shown is; Surrogate marker name,
Anchor marker, the allele that is correlated with risk-allele of the anchor-marker, position
of the surrogate marker in NCBI Build 36, D′, r², and P-value of the correlation
between the markers. Allelic codes are A = 1, C = 2, G = 3, T = 4.

| | | | | | | | Seq |
|---|---|---|---|---|---|---|---|
| | Anchor | | Pos in NCBI | | | | ID |
| Marker | Marker | Allele | Build 36 | D′ | r2 | P-value | No: |
| rs13280181 | rs445114 | 1 | 128355698 | 0.756983 | 0.213711 | 4.00E−06 | 309 |
| rs12707923 | rs445114 | 2 | 128370181 | 0.841519 | 0.246916 | 2.52E−07 | 310 |
| rs6984900 | rs445114 | 4 | 128373451 | 0.841519 | 0.246916 | 2.52E−07 | 311 |
| rs17450865 | rs445114 | 4 | 128376979 | 0.917124 | 0.277119 | 1.15E−08 | 312 |
| rs7822551 | rs445114 | 3 | 128378370 | 1 | 0.307744 | 5.80E−11 | 313 |
| rs12549518 | rs445114 | 1 | 128378773 | 0.709214 | 0.208499 | 7.64E−07 | 314 |
| rs6996866 | rs445114 | 4 | 128379337 | 0.709214 | 0.208499 | 7.64E−07 | 315 |
| rs2007197 | rs445114 | 1 | 128380741 | 0.917124 | 0.277119 | 1.15E−08 | 316 |
| rs283727 | rs445114 | 3 | 128382542 | 0.942783 | 0.368446 | 2.13E−12 | 317 |
| rs283728 | rs445114 | 4 | 128382682 | 0.942783 | 0.368446 | 2.13E−12 | 318 |
| rs283704 | rs445114 | 4 | 128384764 | 1 | 0.372747 | 1.15E−14 | 319 |
| rs283705 | rs445114 | 2 | 128386632 | 0.833374 | 0.298101 | 1.36E−09 | 320 |
| rs10107982 | rs445114 | 4 | 128387937 | 1 | 0.639549 | 1.13E−21 | 321 |
| rs453875 | rs445114 | 2 | 128390593 | 1 | 0.760897 | 7.65E−27 | 322 |
| rs445114 | rs445114 | 4 | 128392363 | 1 | 1 | | 323 |
| rs11785664 | rs445114 | 2 | 128399606 | 1 | 0.346681 | 7.82E−14 | 324 |
| rs622556 | rs445114 | 1 | 128402379 | 1 | 0.475878 | 6.74E−18 | 325 |
| rs452529 | rs445114 | 2 | 128402441 | 1 | 0.475878 | 6.74E−18 | 326 |
| rs13256367 | rs445114 | 1 | 128404082 | 1 | 0.931187 | 1.81E−32 | 327 |
| rs10956356 | rs445114 | 3 | 128404148 | 1 | 0.470039 | 1.93E−17 | 328 |
| *rs10956358 | rs445114 | 1 | 128404428 | 0.837883 | 0.39021 | 2.25E−10 | 329 |
| *rs7008928 | rs445114 | 3 | 128404855 | 1 | 1 | 3.41E−36 | 330 |
| *rs7009077 | rs445114 | 3 | 128404978 | 1 | 1 | 3.51E−34 | 331 |
| rs400818 | rs445114 | 1 | 128405728 | 0.949942 | 0.444287 | 2.26E−14 | 332 |
| rs386883 | rs445114 | 2 | 128406053 | 0.944877 | 0.425146 | 6.48E−13 | 333 |
| rs377649 | rs445114 | 2 | 128406423 | 0.949942 | 0.444287 | 2.26E−14 | 334 |
| rs432470 | rs445114 | 3 | 128408226 | 0.940717 | 0.341857 | 1.49E−11 | 335 |
| rs424281 | rs445114 | 2 | 128408608 | 0.940717 | 0.341857 | 1.49E−11 | 336 |
| rs1668875 | rs445114 | 2 | 128410285 | 0.961538 | 0.7525 | 5.56E−24 | 337 |
| rs7002712 | rs445114 | 4 | 128410794 | 0.937625 | 0.31609 | 9.47E−11 | 338 |
| rs587948 | rs445114 | 1 | 128410862 | 0.922066 | 0.714523 | 3.25E−21 | 339 |
| rs623401 | rs445114 | 3 | 128410909 | 0.924009 | 0.718842 | 4.55E−22 | 340 |
| rs10956359 | rs445114 | 4 | 128411336 | 1 | 0.665653 | 2.16E−22 | 341 |
| rs17464492 | rs445114 | 1 | 128412048 | 1 | 0.639549 | 1.13E−21 | 342 |
| rs420101 | rs445114 | 2 | 128413061 | 0.768011 | 0.480074 | 2.36E−14 | 343 |
| rs7838714 | rs445114 | 2 | 128413130 | 0.805045 | 0.236747 | 1.21E−07 | 344 |
| rs389143 | rs445114 | 3 | 128413562 | 0.762967 | 0.458076 | 9.04E−14 | 345 |
| rs688201 | rs445114 | 2 | 128413584 | 0.762967 | 0.458076 | 9.04E−14 | 346 |
| rs687324 | rs445114 | 1 | 128413773 | 0.762967 | 0.458076 | 9.04E−14 | 347 |
| rs687279 | rs445114 | 3 | 128413806 | 0.554393 | 0.247752 | 1.12E−07 | 348 |
| rs436238 | rs445114 | 3 | 128414210 | 0.762967 | 0.458076 | 9.04E−14 | 349 |
| rs581761 | rs445114 | 2 | 128414413 | 0.758847 | 0.438162 | 3.35E−13 | 350 |
| rs673745 | rs445114 | 3 | 128414451 | 0.762967 | 0.458076 | 9.04E−14 | 351 |
| rs688937 | rs445114 | 1 | 128414563 | 0.762967 | 0.458076 | 9.04E−14 | 352 |
| rs672888 | rs445114 | 4 | 128414645 | 0.762967 | 0.458076 | 9.04E−14 | 353 |
| rs7826557 | rs445114 | 1 | 128414913 | 0.862746 | 0.249308 | 4.36E−08 | 354 |
| rs418269 | rs445114 | 2 | 128415540 | 0.762967 | 0.458076 | 9.04E−14 | 355 |
| rs385278 | rs445114 | 1 | 128416199 | 0.746378 | 0.43909 | 1.49E−12 | 356 |
| rs391640 | rs445114 | 4 | 128416306 | 0.86624 | 0.322364 | 5.88E−09 | 357 |
| rs670725 | rs445114 | 4 | 128416339 | 0.762967 | 0.458076 | 9.04E−14 | 358 |
| rs382824 | rs445114 | 1 | 128416906 | 0.762967 | 0.458076 | 9.04E−14 | 359 |
| rs383205 | rs445114 | 2 | 128417159 | 0.762967 | 0.458076 | 9.04E−14 | 360 |
| rs373616 | rs445114 | 1 | 128417244 | 0.762967 | 0.458076 | 9.04E−14 | 361 |
| rs13275275 | rs445114 | 1 | 128418909 | 0.749036 | 0.431322 | 2.57E−12 | 362 |
| rs13248140 | rs445114 | 3 | 128419070 | 0.762967 | 0.458076 | 9.04E−14 | 363 |
| rs10956361 | rs445114 | 3 | 128419288 | 0.794672 | 0.477399 | 2.50E−14 | 364 |
| rs10956362 | rs445114 | 1 | 128419568 | 0.762967 | 0.458076 | 9.04E−14 | 365 |
| rs13249993 | rs445114 | 3 | 128419697 | 0.814763 | 0.256442 | 3.32E−08 | 366 |
| rs11777532 | rs445114 | 2 | 128419790 | 1 | 0.309995 | 1.20E−12 | 367 |
| rs10956363 | rs445114 | 3 | 128420955 | 0.762967 | 0.458076 | 9.04E−14 | 368 |
| rs4871782 | rs445114 | 3 | 128421416 | 0.762967 | 0.458076 | 9.04E−14 | 369 |
| rs10087810 | rs445114 | 4 | 128421912 | 0.754583 | 0.418968 | 1.18E−12 | 370 |
| rs12541832 | rs445114 | 2 | 128422353 | 0.575952 | 0.287723 | 8.16E−09 | 371 |
| rs13262406 | rs445114 | 1 | 128422921 | 0.545729 | 0.262963 | 1.21E−07 | 372 |
| rs10098985 | rs445114 | 4 | 128424201 | 0.86648 | 0.260284 | 1.74E−08 | 373 |
| rs13281615 | rs445114 | 1 | 128424800 | 0.758847 | 0.438162 | 3.35E−13 | 374 |
| rs13256275 | rs445114 | 3 | 128425408 | 0.772686 | 0.269142 | 6.84E−08 | 375 |
| rs13267780 | rs445114 | 3 | 128426999 | 0.834516 | 0.376761 | 9.26E−11 | 376 |
| rs10447995 | rs445114 | 3 | 128427106 | 0.873822 | 0.284616 | 3.22E−09 | 377 |
| rs7014657 | rs445114 | 3 | 128430423 | 0.850316 | 0.25245 | 2.29E−07 | 378 |
| rs7002826 | rs445114 | 2 | 128433453 | 0.804201 | 0.232532 | 1.66E−07 | 379 |
| rs7007568 | rs445114 | 2 | 128434088 | 0.804201 | 0.232532 | 1.66E−07 | 380 |
| rs7842494 | rs445114 | 1 | 128435752 | 0.931014 | 0.279034 | 1.28E−09 | 381 |
| rs5022926 | rs445114 | 2 | 128436011 | 0.804201 | 0.232532 | 1.66E−07 | 382 |
| rs9693995 | rs445114 | 4 | 128437695 | 0.750165 | 0.400456 | 3.92E−12 | 383 |
| rs2121629 | rs445114 | 4 | 128442209 | 0.750165 | 0.400456 | 3.92E−12 | 384 |
| rs978683 | rs445114 | 2 | 128443299 | 0.70985 | 0.470079 | 8.52E−14 | 385 |
| rs9283954 | rs445114 | 4 | 128444552 | 1 | 0.309995 | 1.20E−12 | 386 |
| rs7831303 | rs445114 | 1 | 128445914 | 0.86648 | 0.260284 | 1.74E−08 | 387 |
| rs7815100 | rs445114 | 2 | 128445983 | 0.804201 | 0.232532 | 1.66E−07 | 388 |
| rs4143118 | rs445114 | 3 | 128446650 | 0.765791 | 0.429784 | 2.13E−11 | 389 |
| rs6988647 | rs445114 | 2 | 128446838 | 0.804201 | 0.232532 | 1.66E−07 | 390 |
| rs9693143 | rs445114 | 4 | 128447207 | 0.804201 | 0.232532 | 1.66E−07 | 391 |
| rs2060775 | rs445114 | 3 | 128447808 | 0.695208 | 0.239227 | 1.23E−06 | 392 |
| rs10956364 | rs445114 | 4 | 128448065 | 0.750165 | 0.400456 | 3.92E−12 | 393 |
| rs11776330 | rs445114 | 4 | 128448145 | 0.765884 | 0.416998 | 2.40E−11 | 394 |
| rs7845452 | rs445114 | 2 | 128448591 | 0.86648 | 0.260284 | 1.74E−08 | 395 |
| rs7815245 | rs445114 | 4 | 128452779 | 0.86648 | 0.260284 | 1.74E−08 | 396 |
| rs2121631 | rs445114 | 2 | 128455738 | 0.69495 | 0.29656 | 1.67E−08 | 397 |
| rs1562430 | rs445114 | 3 | 128457034 | 0.804201 | 0.232532 | 1.66E−07 | 398 |
| rs2392780 | rs445114 | 3 | 128457207 | 0.804201 | 0.232532 | 1.66E−07 | 399 |
| rs7015780 | rs445114 | 4 | 128458689 | 0.862481 | 0.24856 | 3.93E−08 | 400 |

*rs10956358 has alias: rs437980, rs7008928 has alias: rs620861, rs7009077 has alias: rs443053

TABLE 11
Surrogate markers (based on HapMap CEU sample set; http://www.hapmap.org) on
Chromosome 19q13.2 with r² > 0.1 to rs8102476. Shown is; Surrogate
marker name, Anchor marker, the allele that is correlated with risk-allele of the
anchor-marker, position of surrogate marker in NCBI Build 36, and D′, r2, and
P-value of the correlation between the markers. Allelic codes are A = 1, C =
2, G = 3, T = 4.

| | Anchor | | Pos in NCBI | | | | Seq |
|---|---|---|---|---|---|---|---|
| Marker | Marker | Allele | Build 36 | D′ | r2 | P-value | ID No: |
| rs8110367 | rs8102476 | 4 | 43170305 | 0.520759 | 0.130037 | 0.000500 | 401 |
| rs10500278 | rs8102476 | 3 | 43186344 | 0.520759 | 0.130037 | 0.000500 | 402 |
| rs705503 | rs8102476 | 3 | 43206158 | 0.446453 | 0.116540 | 0.000385 | 403 |
| rs1654338 | rs8102476 | 3 | 43228193 | 0.407931 | 0.121790 | 0.001349 | 404 |
| rs4803899 | rs8102476 | 1 | 43419480 | 0.954212 | 0.544559 | 2.20E−15 | 405 |
| rs1036233 | rs8102476 | 1 | 43420054 | 0.495700 | 0.214375 | 0.000014 | 406 |
| rs7246060 | rs8102476 | 3 | 43423502 | 0.550709 | 0.245840 | 2.43E−06 | 407 |
| rs8102476 | rs8102476 | 2 | 43426978 | 1 | 1 | 0 | 408 |
| rs12976534 | rs8102476 | 1 | 43435802 | 1.000.000 | 0.816572 | 1.25E−28 | 409 |
| rs4803934 | rs8102476 | 2 | 43438407 | 1.000.000 | 0.763477 | 4.61E−27 | 410 |
| rs11668070 | rs8102476 | 3 | 43440753 | 1.000.000 | 0.789581 | 1.14E−27 | 411 |
| rs7250689 | rs8102476 | 4 | 43445465 | 1.000.000 | 0.791045 | 2.98E−28 | 412 |
| rs7253245 | rs8102476 | 3 | 43445626 | 0.588838 | 0.139684 | 0.000170 | 413 |
| rs3786870 | rs8102476 | 2 | 43447704 | 0.588838 | 0.139684 | 0.000170 | 414 |
| rs3786872 | rs8102476 | 2 | 43447929 | 1.000.000 | 0.323671 | 5.74E−12 | 415 |
| rs3786877 | rs8102476 | 4 | 43451020 | 0.738167 | 0.471885 | 4.25E−13 | 416 |
| rs12610791 | rs8102476 | 4 | 43453003 | 1.000.000 | 0.153588 | 1.41E−06 | 417 |
| rs8101725 | rs8102476 | 2 | 43456912 | 0.733410 | 0.453316 | 1.17E−12 | 418 |
| rs870218 | rs8102476 | 3 | 43463015 | 0.852096 | 0.121227 | 0.000288 | 419 |
| rs12611009 | rs8102476 | 4 | 43464321 | 0.710742 | 0.426034 | 2.07E−10 | 420 |
| rs3826896 | rs8102476 | 4 | 43465362 | 0.733410 | 0.453316 | 1.17E−12 | 421 |
| rs8104823 | rs8102476 | 1 | 43470457 | 0.848201 | 0.123995 | 0.000466 | 422 |
| rs1821284 | rs8102476 | 2 | 43475421 | 0.574850 | 0.195351 | 0.000041 | 423 |
| rs4802327 | rs8102476 | 3 | 43485159 | 0.882262 | 0.162407 | 0.000032 | 424 |
| rs11672219 | rs8102476 | 3 | 43485436 | 0.882262 | 0.162407 | 0.000032 | 425 |
| rs3816044 | rs8102476 | 3 | 43486590 | 0.882262 | 0.162407 | 0.000032 | 426 |
| rs2304177 | rs8102476 | 3 | 43486999 | 0.877516 | 0.160665 | 0.000064 | 427 |
| rs4312417 | rs8102476 | 1 | 43489029 | 0.603523 | 0.161208 | 0.000170 | 428 |
| rs3178327 | rs8102476 | 1 | 43489926 | 0.602646 | 0.164369 | 0.000151 | 429 |
| rs3900981 | rs8102476 | 3 | 43492005 | 0.914878 | 0.250889 | 1.10E−07 | 430 |
| rs3843754 | rs8102476 | 3 | 43499024 | 0.610834 | 0.164292 | 0.000102 | 431 |
| rs2302182 | rs8102476 | 4 | 43519800 | 0.682463 | 0.110762 | 0.001143 | 432 |
| rs1052375 | rs8102476 | 1 | 43553173 | 0.550831 | 0.265280 | 2.09E−07 | 433 |
| rs12609246 | rs8102476 | 1 | 43601479 | 0.467823 | 0.118355 | 0.001120 | 434 |
| rs3745843 | rs8102476 | 3 | 43624960 | 1.000.000 | 0.153588 | 1.41E−06 | 435 |
| rs3745844 | rs8102476 | 3 | 43634088 | 1.000.000 | 0.180593 | 1.77E−07 | 436 |
| rs2304150 | rs8102476 | 3 | 43647423 | 0.324253 | 0.100289 | 0.002002 | 437 |

### Example 2

Marker rs620861, which is an alias for marker rs7008928, is a surrogate of marker rs445114 (r²=1; Table 10). Investigation of the association of this marker to prostate cancer reveals the following result:

TABLE 12
Association results for rs620861 [G] on 8q24.

| Study | Cases | Controls | Frequency | | | |
|---|---|---|---|---|---|---|
| population | (N) | (N) | Cases | Controls | OR | P-value |
| Iceland^{a} | 1,849 | 35,327 | 0.710 | 0.670 | 1.20 | 6.7E−07 |

In a similar manner, marker rs16902104 is an excellent surrogate for rs16902094 (OR=1.36; P-value 2.32E-10).

The association of surrogate markers was further investigated by imputing markers in the HapMap collection into the Icelandic population. This was done using the IMPUTE software (Marchini, J. et al. Nat Genet 39:906-13 (2007)) and the HapMap (NCBI Build 36 (db126b)) CEU data as reference (Frazer, K. A., et al. Nature 449:851-61 (2007)).

Results of this analysis is shown in the Tables 13-16 below. The association signal for the different surrogate markers is different. This is due to the different degree of linkage disequilibrium between the markers and the anchor marker. Further, since the data shown in Tables 13-16 is based on Icelandic data only (1776 cases and 35675 controls), the association signal is not as strong as it would be for a larger dataset. This leads to a reduced power to detect the association signal associated with each locus.

TABLE 13
Association of surrogate markers of rs10934853 on Chromosome 3q21.3 with Prostate
Cancer. Results are shown for imputed Icelandic data set. Shown is the marker name and position in
NCBI Build 36, the risk allele and its population frequency, number of cases and controls, the Odds
ratio, and P values. Allelic codes are A = 1, C = 2, G = 3 and T = 4.

| | Pos NCBI | Risk | | No of | No of | | | Seq Id |
|---|---|---|---|---|---|---|---|---|
| Marker | B36 | Allele | Freq. | cases | controls | OR | P-Value | NO |
| rs16845806 | 129193164 | A | 0.190533 | 1776 | 35675 | 1.11236 | 0.0265212 | 21 |
| rs7630727 | 129196138 | C | 0.458237 | 1776 | 35675 | 1.10577 | 0.00568714 | 22 |
| rs1549876 | 129197301 | G | 0.468457 | 1776 | 35675 | 1.10543 | 0.00613608 | 23 |
| rs17282209 | 129197886 | C | 0.094161 | 1776 | 35675 | 1.08949 | 0.147235 | 24 |
| rs6439104 | 129200392 | C | 0.187097 | 1776 | 35675 | 1.10633 | 0.0240607 | 25 |
| rs1469659 | 129203430 | T | 0.1871 | 1776 | 35675 | 1.10614 | 0.0242733 | 26 |
| rs7611430 | 129205905 | G | 0.45749 | 1776 | 35675 | 1.10462 | 0.00591018 | 27 |
| rs6770337 | 129207423 | G | 0.457343 | 1776 | 35675 | 1.10449 | 0.00591256 | 28 |
| rs6777095 | 129209327 | A | 0.165982 | 1776 | 35675 | 1.12338 | 0.0114255 | 29 |
| rs4602341 | 129215781 | A | 0.180587 | 1776 | 35675 | 1.10778 | 0.0196696 | 30 |
| rs4857833 | 129228387 | G | 0.420264 | 1776 | 35675 | 1.0996 | 0.00607082 | 31 |
| rs6439108 | 129228455 | G | 0.420264 | 1776 | 35675 | 1.0996 | 0.00607082 | 32 |
| rs6764517 | 129230531 | A | 0.180953 | 1776 | 35675 | 1.10749 | 0.0192157 | 33 |
| rs981447 | 129236378 | G | 0.420264 | 1776 | 35675 | 1.0996 | 0.00607082 | 34 |
| rs981446 | 129236420 | G | 0.420052 | 1772 | 35471 | 1.09835 | 0.00665709 | 35 |
| rs1469658 | 129241904 | T | 0.180953 | 1776 | 35675 | 1.1075 | 0.0192157 | 36 |
| rs2335772 | 129255226 | G | 0.537872 | 1776 | 35675 | 1.08136 | 0.0244486 | 37 |
| rs1030656 | 129256317 | G | 0.420264 | 1776 | 35675 | 1.09961 | 0.00607052 | 38 |
| rs1030655 | 129256366 | T | 0.420264 | 1776 | 35675 | 1.09961 | 0.00607052 | 39 |
| rs2335771 | 129262634 | A | 0.420264 | 1776 | 35675 | 1.09961 | 0.00607054 | 40 |
| rs759945 | 129262772 | G | 0.201428 | 1776 | 35675 | 1.10093 | 0.0257225 | 41 |
| rs2075402 | 129266952 | C | 0.420264 | 1776 | 35675 | 1.09961 | 0.00607046 | 42 |
| rs1554534 | 129282359 | G | 0.180952 | 1776 | 35675 | 1.1075 | 0.0192103 | 43 |
| rs3732402 | 129288908 | G | 0.42132 | 1776 | 35675 | 1.09705 | 0.00801588 | 44 |
| rs13091198 | 129294727 | T | 0.114425 | 1776 | 35675 | 1.07487 | 0.200426 | 45 |
| rs11714052 | 129297147 | A | 0.093033 | 1776 | 35675 | 1.06895 | 0.251576 | 46 |
| rs6439113 | 129299262 | A | 0.179852 | 1776 | 35675 | 1.11915 | 0.0102817 | 47 |
| rs6787614 | 129300168 | A | 0.105028 | 1776 | 35675 | 1.05445 | 0.349432 | 48 |
| rs11720239 | 129300645 | G | 0.092843 | 1776 | 35669 | 1.06769 | 0.259935 | 49 |
| rs11715661 | 129303536 | T | 0.092807 | 1776 | 35675 | 1.06771 | 0.260031 | 50 |
| rs7641133 | 129305010 | T | 0.272084 | 1776 | 35675 | 1.12241 | 0.00240999 | 51 |
| rs11924142 | 129309308 | C | 0.179295 | 1776 | 35675 | 1.12332 | 0.00795035 | 52 |
| rs7650365 | 129316693 | G | 0.543571 | 1776 | 35669 | 1.07651 | 0.0322924 | 53 |
| rs6788879 | 129318319 | A | 0.179295 | 1776 | 35675 | 1.1233 | 0.00796165 | 54 |
| rs6439115 | 129318493 | C | 0.179295 | 1776 | 35675 | 1.1233 | 0.00796223 | 55 |
| rs4857836 | 129318977 | C | 0.272082 | 1776 | 35675 | 1.1224 | 0.00241319 | 56 |
| rs4857837 | 129319009 | A | 0.272082 | 1776 | 35675 | 1.1224 | 0.00241279 | 57 |
| rs11707462 | 129321320 | C | 0.092773 | 1776 | 35675 | 1.06772 | 0.260089 | 58 |
| rs9821568 | 129321689 | G | 0.179296 | 1776 | 35675 | 1.12332 | 0.00795594 | 59 |
| rs6784159 | 129326068 | C | 0.092757 | 1776 | 35675 | 1.06775 | 0.260055 | 60 |
| rs2811475 | 129328391 | C | 0.179299 | 1776 | 35675 | 1.12333 | 0.00794779 | 61 |
| rs13095660 | 129330846 | T | 0.092736 | 1776 | 35675 | 1.06789 | 0.259105 | 62 |
| rs6439116 | 129331757 | C | 0.092735 | 1776 | 35675 | 1.06791 | 0.259033 | 63 |
| rs6414310 | 129339263 | T | 0.272041 | 1776 | 35675 | 1.12247 | 0.00240394 | 64 |
| rs2955102 | 129348200 | G | 0.272018 | 1776 | 35675 | 1.12254 | 0.00239438 | 65 |
| rs11920225 | 129354865 | G | 0.179325 | 1776 | 35675 | 1.12335 | 0.0079397 | 66 |
| rs11709066 | 129357602 | A | 0.092687 | 1776 | 35675 | 1.06791 | 0.259254 | 67 |
| rs11716941 | 129358064 | G | 0.092686 | 1776 | 35675 | 1.06792 | 0.259182 | 68 |
| rs2811472 | 129361561 | G | 0.179328 | 1776 | 35675 | 1.12333 | 0.00794914 | 69 |
| rs13077790 | 129365348 | T | 0.092676 | 1776 | 35675 | 1.06795 | 0.258994 | 71 |
| rs2811473 | 129367627 | C | 0.17933 | 1776 | 35675 | 1.12335 | 0.00793777 | 72 |
| rs2687728 | 129367698 | A | 0.17933 | 1776 | 35675 | 1.12335 | 0.00793829 | 73 |
| rs10934850 | 129369647 | A | 0.092662 | 1776 | 35675 | 1.06786 | 0.259716 | 74 |
| rs872267 | 129370757 | A | 0.272001 | 1776 | 35675 | 1.12253 | 0.00239846 | 75 |
| rs2687731 | 129371384 | T | 0.179331 | 1776 | 35675 | 1.12339 | 0.00792095 | 76 |
| rs3122174 | 129372065 | G | 0.179331 | 1776 | 35675 | 1.12338 | 0.00792176 | 77 |
| rs2999051 | 129372094 | C | 0.392831 | 1776 | 35675 | 1.09533 | 0.00969242 | 78 |
| rs13067650 | 129372232 | A | 0.092649 | 1776 | 35675 | 1.06794 | 0.259221 | 79 |
| rs2248668 | 129373439 | G | 0.185243 | 1776 | 35675 | 1.10942 | 0.0177349 | 80 |
| rs2955121 | 129374086 | G | 0.179333 | 1776 | 35675 | 1.1234 | 0.00791357 | 81 |
| rs11706455 | 129374681 | A | 0.092643 | 1776 | 35675 | 1.06796 | 0.259105 | 82 |
| rs2999052 | 129374727 | C | 0.271983 | 1776 | 35675 | 1.12258 | 0.00239178 | 83 |
| rs11715394 | 129376254 | T | 0.092642 | 1776 | 35675 | 1.06797 | 0.259081 | 84 |
| rs2687729 | 129377916 | G | 0.271979 | 1776 | 35675 | 1.12259 | 0.00238993 | 85 |
| rs2999060 | 129383184 | G | 0.179337 | 1776 | 35675 | 1.12332 | 0.00795103 | 87 |
| rs2999056 | 129386212 | A | 0.179337 | 1776 | 35675 | 1.12333 | 0.00795023 | 88 |
| rs2955123 | 129386368 | C | 0.18846 | 1776 | 35675 | 1.13241 | 0.00465959 | 89 |
| rs2811517 | 129386880 | C | 0.179337 | 1776 | 35675 | 1.12333 | 0.00795015 | 90 |
| rs2811516 | 129388141 | G | 0.285558 | 1776 | 35675 | 1.11547 | 0.00679751 | 91 |
| rs2811515 | 129388208 | A | 0.268018 | 1776 | 35675 | 1.12272 | 0.0023819 | 92 |
| rs2811514 | 129390619 | G | 0.267965 | 1776 | 35675 | 1.1227 | 0.00238181 | 93 |
| rs2811512 | 129394510 | A | 0.267936 | 1776 | 35675 | 1.12263 | 0.00239317 | 94 |
| rs2811511 | 129395678 | A | 0.08737 | 1776 | 35675 | 1.06843 | 0.267016 | 95 |
| rs883238 | 129395953 | G | 0.267937 | 1776 | 35675 | 1.12262 | 0.00239446 | 96 |
| rs940061 | 129396404 | G | 0.180558 | 1776 | 35675 | 1.12308 | 0.00762893 | 97 |
| rs2811510 | 129397202 | G | 0.180558 | 1776 | 35675 | 1.12308 | 0.00762893 | 98 |
| rs2811483 | 129397363 | C | 0.180558 | 1776 | 35675 | 1.12309 | 0.00762893 | 99 |
| rs2811484 | 129397543 | A | 0.180558 | 1776 | 35675 | 1.12309 | 0.00762893 | 100 |
| rs2687730 | 129398147 | T | 0.08737 | 1776 | 35675 | 1.06843 | 0.267016 | 101 |
| rs2811509 | 129399241 | A | 0.267937 | 1776 | 35675 | 1.12262 | 0.00239447 | 102 |
| rs2492285 | 129400690 | A | 0.267936 | 1776 | 35675 | 1.12262 | 0.00239433 | 103 |
| rs2687720 | 129401645 | T | 0.267936 | 1776 | 35675 | 1.12262 | 0.00239433 | 104 |
| rs2811508 | 129402713 | C | 0.267937 | 1776 | 35675 | 1.12262 | 0.00239465 | 105 |
| rs2811486 | 129402765 | G | 0.08737 | 1776 | 35675 | 1.06843 | 0.267016 | 106 |
| rs6439119 | 129405877 | T | 0.267937 | 1776 | 35675 | 1.12262 | 0.00239465 | 107 |
| rs2955125 | 129407882 | G | 0.180558 | 1776 | 35675 | 1.12308 | 0.00763054 | 108 |
| rs2955126 | 129408944 | A | 0.08932 | 1776 | 35675 | 1.08166 | 0.189446 | 109 |
| rs2955127 | 129409206 | G | 0.180558 | 1776 | 35675 | 1.12308 | 0.00763055 | 110 |
| rs2955129 | 129411897 | T | 0.08737 | 1776 | 35675 | 1.06843 | 0.267016 | 112 |
| rs7374072 | 129413556 | C | 0.267937 | 1776 | 35675 | 1.12261 | 0.0023954 | 113 |
| rs2999090 | 129414030 | G | 0.08737 | 1776 | 35675 | 1.06843 | 0.267016 | 114 |
| rs7372439 | 129414092 | A | 0.267937 | 1776 | 35675 | 1.12261 | 0.0023954 | 115 |
| rs4857871 | 129416476 | C | 0.267937 | 1776 | 35675 | 1.12261 | 0.0023954 | 116 |
| rs4857872 | 129416512 | T | 0.267937 | 1776 | 35675 | 1.12261 | 0.0023954 | 117 |
| rs4857873 | 129416908 | A | 0.267937 | 1776 | 35675 | 1.12261 | 0.0023954 | 118 |
| rs6770140 | 129417019 | T | 0.267937 | 1776 | 35675 | 1.12261 | 0.0023954 | 119 |
| rs4384971 | 129417464 | C | 0.267937 | 1776 | 35675 | 1.12261 | 0.0023954 | 120 |
| rs2999089 | 129417849 | C | 0.08737 | 1776 | 35675 | 1.06843 | 0.267016 | 121 |
| rs6439121 | 129418151 | G | 0.268537 | 1776 | 35675 | 1.12113 | 0.00270169 | 122 |
| rs2254379 | 129420016 | C | 0.180443 | 1773 | 35657 | 1.12021 | 0.00918407 | 123 |
| rs2955130 | 129420504 | G | 0.08737 | 1776 | 35675 | 1.06843 | 0.267003 | 124 |
| rs9814834 | 129420827 | G | 0.267938 | 1776 | 35675 | 1.12261 | 0.00239555 | 125 |
| rs2955132 | 129422916 | C | 0.180559 | 1776 | 35675 | 1.12307 | 0.00763298 | 126 |
| rs9845651 | 129423043 | T | 0.267938 | 1776 | 35675 | 1.12261 | 0.00239583 | 127 |
| rs6439122 | 129423224 | A | 0.267938 | 1776 | 35675 | 1.12261 | 0.00239583 | 128 |
| rs9873786 | 129425069 | A | 0.267938 | 1776 | 35675 | 1.12261 | 0.00239597 | 129 |
| rs4857838 | 129426018 | A | 0.267938 | 1776 | 35675 | 1.12261 | 0.00239597 | 130 |
| rs6775988 | 129427167 | C | 0.267938 | 1776 | 35675 | 1.12261 | 0.00239598 | 131 |
| rs9830294 | 129429478 | G | 0.267938 | 1776 | 35675 | 1.12261 | 0.00239598 | 132 |
| rs4857877 | 129430750 | A | 0.267938 | 1776 | 35675 | 1.12261 | 0.00239598 | 133 |
| rs2999086 | 129433938 | C | 0.087366 | 1776 | 35675 | 1.06842 | 0.267076 | 134 |
| rs2999085 | 129434249 | G | 0.087366 | 1776 | 35675 | 1.06842 | 0.267063 | 135 |
| rs2999084 | 129435178 | A | 0.087365 | 1776 | 35675 | 1.06843 | 0.267039 | 136 |
| rs2999083 | 129437124 | C | 0.087365 | 1776 | 35675 | 1.06843 | 0.267023 | 137 |
| rs2999081 | 129438962 | A | 0.077497 | 1776 | 35675 | 1.07185 | 0.295545 | 138 |
| rs2999079 | 129439922 | T | 0.087363 | 1776 | 35675 | 1.06846 | 0.266852 | 139 |
| rs4074440 | 129440703 | G | 0.267938 | 1776 | 35675 | 1.12261 | 0.00239596 | 140 |
| rs9843281 | 129444086 | A | 0.267938 | 1776 | 35675 | 1.12261 | 0.00239611 | 142 |
| rs2999073 | 129445019 | G | 0.087359 | 1776 | 35675 | 1.06852 | 0.266459 | 143 |
| rs2955085 | 129445447 | G | 0.087359 | 1776 | 35675 | 1.06852 | 0.266433 | 144 |
| rs2999072 | 129445566 | G | 0.087359 | 1776 | 35675 | 1.06852 | 0.266433 | 145 |
| rs13434079 | 129446138 | A | 0.267938 | 1776 | 35675 | 1.12261 | 0.00239625 | 146 |
| rs2955088 | 129446400 | C | 0.087358 | 1776 | 35675 | 1.06853 | 0.266368 | 147 |
| rs2999070 | 129447341 | G | 0.087358 | 1776 | 35675 | 1.06854 | 0.266317 | 148 |
| rs17343355 | 129449481 | C | 0.541248 | 1776 | 35675 | 1.07647 | 0.0322715 | 149 |
| rs2955090 | 129452061 | T | 0.087357 | 1776 | 35675 | 1.06852 | 0.266496 | 150 |
| rs2955091 | 129454051 | G | 0.087357 | 1776 | 35675 | 1.06852 | 0.266485 | 151 |
| rs2999069 | 129454299 | C | 0.087357 | 1776 | 35675 | 1.06852 | 0.266485 | 152 |
| rs2955092 | 129455218 | C | 0.087357 | 1776 | 35675 | 1.06852 | 0.266473 | 153 |
| rs2955094 | 129459613 | A | 0.087356 | 1776 | 35675 | 1.06853 | 0.266409 | 154 |
| rs2955095 | 129460355 | A | 0.087356 | 1776 | 35675 | 1.06853 | 0.266413 | 155 |
| rs2955096 | 129460556 | A | 0.087356 | 1776 | 35675 | 1.06853 | 0.266401 | 156 |
| rs2999068 | 129460668 | T | 0.087356 | 1776 | 35675 | 1.06853 | 0.266401 | 157 |
| rs2999067 | 129461221 | C | 0.087356 | 1776 | 35675 | 1.06853 | 0.266387 | 158 |
| rs2955099 | 129462344 | G | 0.087356 | 1776 | 35675 | 1.06853 | 0.266374 | 159 |
| rs2999066 | 129462976 | T | 0.087356 | 1776 | 35675 | 1.06853 | 0.266373 | 160 |
| rs2999065 | 129464128 | A | 0.087356 | 1776 | 35675 | 1.06854 | 0.266373 | 161 |
| rs2811545 | 129465838 | C | 0.087356 | 1776 | 35675 | 1.06854 | 0.266372 | 162 |
| rs2999035 | 129465899 | T | 0.163903 | 1776 | 35675 | 1.13152 | 0.00788934 | 163 |
| rs2811544 | 129466185 | T | 0.087356 | 1776 | 35675 | 1.06853 | 0.266372 | 164 |
| rs2811543 | 129466486 | T | 0.087356 | 1776 | 35675 | 1.06853 | 0.266371 | 165 |
| rs2811541 | 129467484 | C | 0.087356 | 1776 | 35675 | 1.06853 | 0.266358 | 166 |
| rs2811540 | 129468259 | T | 0.08477 | 1776 | 35675 | 1.07382 | 0.245707 | 167 |
| rs2811539 | 129469163 | C | 0.08477 | 1776 | 35675 | 1.07383 | 0.245672 | 168 |
| rs2811538 | 129469321 | A | 0.087355 | 1776 | 35675 | 1.06854 | 0.266325 | 169 |
| rs2811396 | 129470046 | A | 0.087355 | 1776 | 35675 | 1.06854 | 0.266313 | 170 |
| rs2811400 | 129470557 | G | 0.087354 | 1776 | 35675 | 1.06851 | 0.266514 | 171 |
| rs2811537 | 129470614 | A | 0.087354 | 1776 | 35675 | 1.06851 | 0.266514 | 172 |
| rs2999064 | 129470930 | G | 0.188851 | 1776 | 35675 | 1.11749 | 0.0130514 | 173 |
| rs2811536 | 129471609 | A | 0.087354 | 1776 | 35675 | 1.06852 | 0.266466 | 174 |
| rs2811534 | 129473798 | A | 0.084769 | 1776 | 35675 | 1.07381 | 0.245772 | 175 |
| rs2811413 | 129473839 | C | 0.321814 | 1776 | 35675 | 1.1015 | 0.00959693 | 176 |
| rs2811415 | 129474217 | A | 0.141246 | 1776 | 35675 | 1.02292 | 0.66147 | 177 |
| rs2811533 | 129474419 | G | 0.087354 | 1776 | 35675 | 1.06853 | 0.266438 | 178 |
| rs2811416 | 129474628 | T | 0.087354 | 1776 | 35675 | 1.06853 | 0.266436 | 179 |
| rs2811532 | 129474860 | G | 0.087353 | 1776 | 35675 | 1.06853 | 0.266411 | 180 |
| rs2811531 | 129475185 | G | 0.087353 | 1776 | 35675 | 1.06853 | 0.266411 | 181 |
| rs2955100 | 129476015 | G | 0.087353 | 1776 | 35675 | 1.06853 | 0.266374 | 182 |
| rs2999061 | 129476313 | A | 0.087353 | 1776 | 35675 | 1.06853 | 0.266373 | 183 |
| rs2811529 | 129476850 | G | 0.267938 | 1776 | 35675 | 1.12261 | 0.00239619 | 184 |
| rs2811527 | 129477294 | A | 0.087353 | 1776 | 35675 | 1.06854 | 0.266361 | 185 |
| rs2811373 | 129480119 | T | 0.087352 | 1776 | 35675 | 1.06851 | 0.266548 | 186 |
| rs2811525 | 129482120 | T | 0.087352 | 1776 | 35675 | 1.06851 | 0.266535 | 187 |
| rs7374952 | 129484057 | T | 0.087352 | 1776 | 35675 | 1.06851 | 0.266511 | 188 |
| rs7374227 | 129484205 | G | 0.087352 | 1776 | 35675 | 1.06851 | 0.266511 | 189 |
| rs4593050 | 129487221 | T | 0.087351 | 1776 | 35675 | 1.06853 | 0.266462 | 190 |
| rs6439124 | 129490156 | A | 0.087351 | 1776 | 35675 | 1.06853 | 0.266396 | 191 |
| rs7373998 | 129490913 | A | 0.08735 | 1776 | 35675 | 1.06854 | 0.26637 | 192 |
| rs2955101 | 129492302 | A | 0.08735 | 1776 | 35675 | 1.06854 | 0.266357 | 193 |
| rs2811519 | 129495566 | T | 0.08735 | 1776 | 35675 | 1.06855 | 0.266318 | 194 |
| rs2811518 | 129496335 | G | 0.087191 | 1775 | 35620 | 1.06847 | 0.266718 | 195 |
| rs2955103 | 129497926 | C | 0.389385 | 1776 | 35675 | 1.09627 | 0.00862498 | 196 |
| rs2811388 | 129501137 | A | 0.083826 | 1771 | 35622 | 1.07352 | 0.242449 | 197 |
| rs2999036 | 129503391 | G | 0.086605 | 1776 | 35675 | 1.07312 | 0.239556 | 198 |
| rs2811390 | 129504171 | T | 0.086605 | 1776 | 35675 | 1.07312 | 0.239556 | 199 |
| rs2811391 | 129505058 | A | 0.086605 | 1776 | 35675 | 1.07312 | 0.239545 | 200 |
| rs2811393 | 129506689 | T | 0.086604 | 1776 | 35675 | 1.07312 | 0.239522 | 201 |
| rs2037965 | 129507734 | C | 0.086604 | 1776 | 35675 | 1.07312 | 0.239496 | 202 |
| rs2811397 | 129509927 | C | 0.087751 | 1776 | 35675 | 1.07387 | 0.233841 | 203 |
| rs6805582 | 129511698 | A | 0.086604 | 1776 | 35675 | 1.07313 | 0.239481 | 204 |
| rs6805621 | 129511894 | T | 0.086604 | 1776 | 35675 | 1.07313 | 0.239481 | 205 |
| rs6794591 | 129513909 | T | 0.086603 | 1776 | 35675 | 1.07313 | 0.239431 | 206 |
| rs16843876 | 129515230 | G | 0.086603 | 1776 | 35675 | 1.07314 | 0.239422 | 207 |
| rs11706852 | 129515577 | A | 0.086603 | 1776 | 35675 | 1.07314 | 0.239422 | 208 |
| rs11706826 | 129515681 | T | 0.086603 | 1776 | 35675 | 1.07314 | 0.239419 | 209 |
| rs11706908 | 129515738 | A | 0.086603 | 1776 | 35675 | 1.07314 | 0.239415 | 210 |
| rs6771646 | 129517225 | G | 0.086602 | 1776 | 35675 | 1.07315 | 0.239327 | 211 |
| rs13095166 | 129519476 | T | 0.184693 | 1776 | 35675 | 1.10785 | 0.0179989 | 212 |
| rs12486127 | 129521379 | A | 0.18472 | 1776 | 35675 | 1.10776 | 0.0180714 | 214 |
| rs12486156 | 129521524 | T | 0.18472 | 1776 | 35675 | 1.10776 | 0.0180749 | 215 |
| rs6772407 | 129527062 | T | 0.084347 | 1776 | 35675 | 1.08604 | 0.170941 | 217 |
| rs4857841 | 129529333 | A | 0.269072 | 1776 | 35675 | 1.11837 | 0.00330785 | 218 |
| rs11710704 | 129529926 | A | 0.084347 | 1776 | 35675 | 1.08603 | 0.170941 | 219 |
| rs16844002 | 129536177 | T | 0.084347 | 1776 | 35675 | 1.08604 | 0.170941 | 220 |
| rs6798749 | 129539587 | A | 0.084122 | 1774 | 35654 | 1.08273 | 0.18854 | 221 |
| rs1735558 | 129542300 | A | 0.184306 | 1775 | 35660 | 1.11133 | 0.0147986 | 222 |
| rs4857879 | 129546808 | C | 0.184722 | 1776 | 35675 | 1.10776 | 0.0180674 | 223 |
| rs11721213 | 129550131 | T | 0.08435 | 1776 | 35675 | 1.08598 | 0.171185 | 224 |
| rs1735549 | 129554499 | T | 0.241217 | 1776 | 35675 | 1.12366 | 0.00333846 | 225 |
| rs1735546 | 129558088 | G | 0.249115 | 1776 | 35675 | 1.12305 | 0.00316979 | 226 |
| rs12632366 | 129560248 | G | 0.557623 | 1776 | 35675 | 1.06225 | 0.0815991 | 227 |
| rs1735545 | 129563950 | C | 0.241369 | 1776 | 35675 | 1.12191 | 0.00367153 | 228 |
| rs1702122 | 129566022 | G | 0.249153 | 1776 | 35675 | 1.11859 | 0.00437024 | 229 |
| rs1108313 | 129567780 | G | 0.556413 | 1776 | 35671 | 1.05719 | 0.10755 | 230 |
| rs1735538 | 129574792 | G | 0.267842 | 1776 | 35675 | 1.10809 | 0.00899775 | 231 |
| rs1702119 | 129577183 | C | 0.24994 | 1776 | 35675 | 1.11533 | 0.00529074 | 232 |
| rs1702118 | 129577968 | G | 0.168305 | 1776 | 35675 | 1.10834 | 0.0223525 | 233 |
| rs3021461 | 129578342 | C | 0.240891 | 1774 | 35660 | 1.11479 | 0.00585462 | 234 |
| rs2977565 | 129578457 | A | 0.239959 | 1776 | 35675 | 1.11772 | 0.00491973 | 235 |
| rs2293947 | 129580186 | C | 0.071154 | 1771 | 35627 | 1.08951 | 0.188219 | 236 |
| rs741925 | 129592606 | T | 0.160768 | 1776 | 35675 | 1.11178 | 0.0206329 | 237 |
| rs729847 | 129593460 | A | 0.247283 | 1776 | 35675 | 1.12289 | 0.0033416 | 238 |
| rs1702134 | 129593891 | T | 0.169509 | 1776 | 35675 | 1.11456 | 0.0180199 | 239 |
| rs1620440 | 129594997 | C | 0.159376 | 1776 | 35667 | 1.11124 | 0.0210842 | 240 |
| rs7632169 | 129597277 | C | 0.257379 | 1776 | 35675 | 1.11997 | 0.00375256 | 241 |
| rs1735527 | 129598071 | G | 0.159639 | 1776 | 35675 | 1.11269 | 0.0202305 | 242 |
| rs760383 | 129602255 | G | 0.248549 | 1776 | 35675 | 1.12069 | 0.00393357 | 243 |
| rs6780368 | 129604729 | T | 0.159773 | 1776 | 35675 | 1.11388 | 0.0193951 | 247 |
| rs2659685 | 129605086 | A | 0.256975 | 1776 | 35675 | 1.11975 | 0.00382081 | 248 |
| rs1735537 | 129605510 | C | 0.256501 | 1770 | 35645 | 1.11369 | 0.00551919 | 250 |
| rs2977564 | 129606476 | G | 0.597592 | 1776 | 35675 | 1.07805 | 0.0401501 | 252 |

TABLE 14
Association of surrogate markers of rs445114 on Chromosome 8q24.21 with Prostate
Cancer. Results are shown for imputed Icelandic data set. Shown is the marker name and position in
NCBI Build 36, the risk allele and it's frequency in the population, number of cases and controls, the
Odds ratio, and P values. Allelic codes are A = 1, C = 2, G = 3 and T = 4.

| | Pos NCBI | Risk | | No of | No of | | | Seq Id |
|---|---|---|---|---|---|---|---|---|
| Marker | B36 | Allele | Freq. | cases | controls | OR | P-Value | NO |
| rs453875 | 128390593 | G | 0.604809 | 1776 | 35675 | 1.19943 | 6.12E−07 | 276 |
| rs10107982 | 128387937 | T | 0.744662 | 1776 | 35675 | 1.22938 | 9.25E−07 | 321 |
| rs13256367 | 128404082 | A | 0.696145 | 1776 | 35675 | 1.20773 | 1.20E−06 | 327 |
| rs1668875 | 128410285 | G | 0.635214 | 1776 | 35675 | 1.1798 | 7.14E−06 | 288 |
| rs587948 | 128410862 | T | 0.636052 | 1776 | 35675 | 1.17937 | 7.49E−06 | 290 |
| rs623401 | 128410909 | C | 0.636033 | 1776 | 35675 | 1.17934 | 7.50E−06 | 291 |
| rs10956359 | 128411336 | T | 0.735687 | 1776 | 35675 | 1.19668 | 8.95E−06 | 341 |
| rs17464492 | 128412048 | A | 0.735493 | 1774 | 35656 | 1.18924 | 1.51E−05 | 342 |
| rs7822551 | 128378370 | G | 0.862764 | 1776 | 35675 | 1.21895 | 0.0002054 | 313 |
| rs17450865 | 128376979 | T | 0.859502 | 1776 | 35675 | 1.21444 | 0.000228732 | 312 |
| rs2007197 | 128380741 | T | 0.860989 | 1776 | 35642 | 1.20942 | 0.000257467 | 316 |
| rs6984900 | 128373451 | T | 0.81559 | 1775 | 35563 | 1.17716 | 0.000381047 | 311 |
| rs12707923 | 128370181 | C | 0.815108 | 1776 | 35675 | 1.17284 | 0.000520043 | 310 |
| rs13280181 | 128355698 | A | 0.811443 | 1776 | 35675 | 1.15179 | 0.00214344 | 309 |
| rs13262081 | 128353948 | G | 0.811649 | 1776 | 35675 | 1.15123 | 0.00232655 | 883 |
| rs391640 | 128416306 | A | 0.814053 | 1776 | 35675 | 1.13303 | 0.00641126 | 357 |
| rs13267780 | 128426999 | G | 0.786019 | 1776 | 35675 | 1.12409 | 0.00701101 | 376 |
| rs581761 | 128414413 | G | 0.642578 | 1776 | 35675 | 1.05484 | 0.144439 | 350 |
| rs389143 | 128413562 | C | 0.649348 | 1776 | 35675 | 1.05037 | 0.176442 | 345 |
| rs688201 | 128413584 | G | 0.649348 | 1776 | 35675 | 1.05037 | 0.176442 | 346 |
| rs687324 | 128413773 | T | 0.649348 | 1776 | 35675 | 1.05038 | 0.176442 | 347 |
| rs436238 | 128414210 | C | 0.649349 | 1776 | 35675 | 1.05037 | 0.176488 | 349 |
| rs10956363 | 128420955 | G | 0.649349 | 1776 | 35675 | 1.05037 | 0.17649 | 368 |
| rs673745 | 128414451 | C | 0.649349 | 1776 | 35675 | 1.05037 | 0.176494 | 351 |
| rs4871782 | 128421416 | G | 0.649349 | 1776 | 35675 | 1.05037 | 0.176495 | 369 |
| rs418269 | 128415540 | G | 0.649349 | 1776 | 35675 | 1.05037 | 0.176496 | 355 |
| rs383205 | 128417159 | G | 0.649349 | 1776 | 35675 | 1.05037 | 0.176496 | 360 |
| rs373616 | 128417244 | T | 0.649349 | 1776 | 35675 | 1.05037 | 0.176496 | 361 |
| rs688937 | 128414563 | T | 0.64935 | 1776 | 35675 | 1.05037 | 0.176501 | 352 |
| rs385278 | 128416199 | T | 0.64935 | 1776 | 35675 | 1.05037 | 0.176501 | 356 |
| rs670725 | 128416339 | A | 0.64935 | 1776 | 35675 | 1.05037 | 0.176501 | 358 |
| rs382824 | 128416906 | T | 0.64935 | 1776 | 35675 | 1.05037 | 0.176501 | 359 |
| rs13275275 | 128418909 | A | 0.64935 | 1776 | 35675 | 1.05037 | 0.176501 | 362 |
| rs13248140 | 128419070 | G | 0.64935 | 1776 | 35675 | 1.05037 | 0.176501 | 363 |
| rs10956361 | 128419288 | G | 0.64935 | 1776 | 35675 | 1.05037 | 0.176501 | 364 |
| rs10956362 | 128419568 | A | 0.64935 | 1776 | 35675 | 1.05037 | 0.176501 | 365 |
| rs12549518 | 128378773 | G | 0.539877 | 1776 | 35675 | 1.04843 | 0.176512 | 314 |
| rs6996866 | 128379337 | C | 0.539792 | 1776 | 35675 | 1.04834 | 0.177413 | 315 |
| rs672888 | 128414645 | A | 0.649119 | 1772 | 35631 | 1.04803 | 0.197236 | 353 |
| rs420101 | 128413061 | G | 0.647409 | 1776 | 35675 | 1.04656 | 0.217992 | 343 |
| rs13281615 | 128424800 | A | 0.638485 | 1776 | 35675 | 1.0419 | 0.258421 | 374 |
| rs11785664 | 128399606 | T | 0.561559 | 1776 | 35675 | 1.03871 | 0.281956 | 278 |
| rs10447995 | 128427106 | G | 0.497839 | 1776 | 35675 | 1.0365 | 0.301301 | 377 |
| rs687279 | 128413806 | C | 0.720176 | 1772 | 35455 | 1.04014 | 0.304734 | 348 |
| rs13262406 | 128422921 | A | 0.713993 | 1776 | 35675 | 1.03936 | 0.310541 | 372 |
| rs12541832 | 128422353 | C | 0.713987 | 1776 | 35675 | 1.03955 | 0.310906 | 371 |
| rs7002712 | 128410794 | A | 0.543458 | 1776 | 35675 | 1.03598 | 0.311933 | 289 |
| rs13249993 | 128419697 | G | 0.502218 | 1776 | 35675 | 1.03298 | 0.35099 | 366 |
| rs11777532 | 128419790 | C | 0.411987 | 1776 | 35675 | 1.02219 | 0.536003 | 367 |
| rs283705 | 128386632 | T | 0.484786 | 1776 | 35675 | 1.01694 | 0.637045 | 274 |
| rs10087810 | 128421912 | T | 0.599327 | 1776 | 35675 | 1.01627 | 0.64928 | 370 |
| rs7842494 | 128435752 | A | 0.410173 | 1776 | 35675 | 1.016 | 0.650896 | 381 |
| rs9283954 | 128444552 | T | 0.409832 | 1776 | 35675 | 1.01596 | 0.652013 | 386 |
| rs13256275 | 128425408 | G | 0.519748 | 1776 | 35675 | 1.01292 | 0.724173 | 295 |
| rs7838714 | 128413130 | T | 0.550952 | 1776 | 35675 | 1.01142 | 0.751477 | 344 |
| rs978683 | 128443299 | A | 0.361727 | 1776 | 35675 | 1.01086 | 0.764541 | 385 |
| rs432470 | 128408226 | T | 0.496537 | 1776 | 35675 | 1.00706 | 0.837803 | 284 |
| rs7015780 | 128458689 | C | 0.558571 | 1776 | 35675 | 1.00677 | 0.846466 | 307 |
| rs424281 | 128408608 | A | 0.496483 | 1774 | 35333 | 1.00645 | 0.851103 | 285 |
| rs10098985 | 128424201 | T | 0.446876 | 1776 | 35675 | 1.00653 | 0.852075 | 373 |
| rs377649 | 128406423 | T | 0.439458 | 1776 | 35675 | 1.00642 | 0.854944 | 283 |
| rs386883 | 128406053 | A | 0.439405 | 1776 | 35675 | 1.00614 | 0.861254 | 282 |
| rs400818 | 128405728 | C | 0.439358 | 1776 | 35675 | 1.00589 | 0.866806 | 281 |
| rs7826557 | 128414913 | A | 0.448814 | 1776 | 35675 | 1.00523 | 0.881329 | 354 |
| rs283727 | 128382542 | A | 0.505028 | 1776 | 35675 | 1.00475 | 0.89419 | 317 |
| rs283728 | 128382682 | A | 0.505064 | 1776 | 35675 | 1.00469 | 0.895472 | 318 |
| rs2121629 | 128442209 | C | 0.413795 | 1776 | 35675 | 1.00438 | 0.899214 | 384 |
| rs283704 | 128384764 | A | 0.47441 | 1776 | 35675 | 1.00449 | 0.899924 | 273 |
| rs4143118 | 128446650 | A | 0.413125 | 1776 | 35675 | 1.00432 | 0.90219 | 389 |
| rs11776330 | 128448145 | G | 0.413158 | 1776 | 35675 | 1.00417 | 0.905673 | 394 |
| rs10956364 | 128448065 | C | 0.413159 | 1776 | 35675 | 1.00413 | 0.906654 | 393 |
| rs2392780 | 128457207 | G | 0.447304 | 1776 | 35675 | 1.00358 | 0.917411 | 399 |
| rs7815245 | 128452779 | T | 0.447022 | 1776 | 35675 | 1.00332 | 0.923576 | 396 |
| rs1562430 | 128457034 | C | 0.447396 | 1774 | 35666 | 1.00315 | 0.927443 | 398 |
| rs7845452 | 128448591 | C | 0.44702 | 1776 | 35675 | 1.00312 | 0.928049 | 395 |
| rs7002826 | 128433453 | C | 0.446808 | 1776 | 35675 | 1.00309 | 0.928786 | 379 |
| rs9693143 | 128447207 | T | 0.447111 | 1776 | 35675 | 1.00289 | 0.933363 | 391 |
| rs7007568 | 128434088 | C | 0.446781 | 1776 | 35675 | 1.00287 | 0.933834 | 380 |
| rs6988647 | 128446838 | C | 0.44702 | 1776 | 35675 | 1.00283 | 0.934878 | 390 |
| rs7815100 | 128445983 | C | 0.446941 | 1776 | 35675 | 1.00278 | 0.936001 | 388 |
| rs7831303 | 128445914 | A | 0.446501 | 1776 | 35675 | 1.00272 | 0.937251 | 387 |
| rs5022926 | 128436011 | C | 0.446758 | 1776 | 35675 | 1.00267 | 0.93852 | 382 |
| rs7014657 | 128430423 | G | 0.448956 | 1776 | 35675 | 1.00235 | 0.946569 | 378 |
| rs9693995 | 128437695 | T | 0.581987 | 1765 | 35363 | 1.00195 | 0.95563 | 383 |
| rs622556 | 128402379 | C | 0.45814 | 1776 | 35675 | 1.00141 | 0.967971 | 279 |
| rs452529 | 128402441 | C | 0.458148 | 1776 | 35675 | 1.00137 | 0.968687 | 280 |

TABLE 15
Association of surrogate markers of rs16902094 on Chromosome 8q24.21 with Prostate
Cancer. Results are shown for imputed Icelandic data set. Shown is the marker name and position
in NCBI Build 36, the risk allele and it's frequency in teh population, number of cases and
controls, the Odds ratio, and P values. Allelic codes are A = 1, C = 2, G = 3 and T = 4.

| | Pos NCBI | Risk | | No of | No of | | | Seq Id |
|---|---|---|---|---|---|---|---|---|
| Marker | B36 | Allele | Freq. | cases | controls | OR | P-Value | NO |
| rs16902103 | 128409556 | C | 0.131813 | 1776 | 35675 | 1.36387 | 1.76E−10 | 286 |
| rs13251915 | 128377137 | T | 0.251144 | 1776 | 35675 | 1.27506 | 3.32E−10 | 271 |
| rs453875 | 128390593 | G | 0.604809 | 1776 | 35675 | 1.19943 | 6.12E−07 | 276 |
| rs283720 | 128379147 | A | 0.274893 | 1775 | 35645 | 1.20581 | 6.28E−07 | 272 |
| rs1668875 | 128410285 | G | 0.635214 | 1776 | 35675 | 1.1798 | 7.14E−06 | 288 |
| rs587948 | 128410862 | T | 0.636052 | 1776 | 35675 | 1.17937 | 7.49E−06 | 290 |
| rs623401 | 128410909 | C | 0.636033 | 1776 | 35675 | 1.17934 | 7.50E−06 | 291 |
| rs11785664 | 128399606 | T | 0.561559 | 1776 | 35675 | 1.03871 | 0.281956 | 278 |
| rs7002712 | 128410794 | A | 0.543458 | 1776 | 35675 | 1.03598 | 0.311933 | 289 |
| rs16902118 | 128417799 | G | 0.147101 | 1776 | 35675 | 1.02578 | 0.607668 | 292 |
| rs283705 | 128386632 | T | 0.484786 | 1776 | 35675 | 1.01694 | 0.637045 | 274 |
| rs11774907 | 128453272 | T | 0.867662 | 1776 | 35675 | 1.02214 | 0.672607 | 305 |
| rs13256275 | 128425408 | G | 0.519748 | 1776 | 35675 | 1.01292 | 0.724173 | 295 |
| rs11782735 | 128435786 | C | 0.859779 | 1776 | 35672 | 1.01758 | 0.725966 | 300 |
| rs10095860 | 128423967 | C | 0.319759 | 1776 | 35675 | 1.01232 | 0.741499 | 293 |
| rs11776260 | 128451670 | A | 0.857308 | 1776 | 35675 | 1.0162 | 0.746102 | 304 |
| rs11784125 | 128449102 | A | 0.860193 | 1776 | 35675 | 1.01509 | 0.764269 | 303 |
| rs11782693 | 128435626 | C | 0.860078 | 1776 | 35675 | 1.01499 | 0.764883 | 298 |
| rs11782700 | 128435678 | C | 0.860078 | 1776 | 35675 | 1.01499 | 0.764883 | 299 |
| rs11774827 | 128434523 | A | 0.860079 | 1776 | 35675 | 1.01499 | 0.764926 | 297 |
| rs11785277 | 128434265 | T | 0.860079 | 1776 | 35675 | 1.01486 | 0.764956 | 296 |
| rs11783559 | 128436107 | C | 0.86009 | 1776 | 35675 | 1.01488 | 0.766454 | 301 |
| rs11783615 | 128436189 | G | 0.860094 | 1776 | 35675 | 1.01475 | 0.766568 | 302 |
| rs16902127 | 128453599 | T | 0.862712 | 1776 | 35675 | 1.01511 | 0.768103 | 306 |
| rs731900 | 128459842 | A | 0.178725 | 1776 | 35667 | 1.0105 | 0.817111 | 308 |
| rs432470 | 128408226 | T | 0.496537 | 1776 | 35675 | 1.00706 | 0.837803 | 284 |
| rs7015780 | 128458689 | C | 0.558571 | 1776 | 35675 | 1.00677 | 0.846466 | 307 |
| rs424281 | 128408608 | A | 0.496483 | 1774 | 35333 | 1.00645 | 0.851103 | 285 |
| rs377649 | 128406423 | T | 0.439458 | 1776 | 35675 | 1.00642 | 0.854944 | 283 |
| rs386883 | 128406053 | A | 0.439405 | 1776 | 35675 | 1.00614 | 0.861254 | 282 |
| rs400818 | 128405728 | C | 0.439358 | 1776 | 35675 | 1.00589 | 0.866806 | 281 |
| rs283704 | 128384764 | A | 0.47441 | 1776 | 35675 | 1.00449 | 0.899924 | 273 |
| rs16902121 | 128424100 | A | 0.140578 | 1776 | 35675 | 1.00353 | 0.944809 | 294 |
| rs622556 | 128402379 | C | 0.45814 | 1776 | 35675 | 1.00141 | 0.967971 | 279 |
| rs452529 | 128402441 | C | 0.458148 | 1776 | 35675 | 1.00137 | 0.968687 | 280 |

TABLE 16
Association of surrogate markers of rs8102476 on Chromosome 19q13.2 with Prostate
Cancer. Results are shown for imputed Icelandic data set. Shown is the marker name and
position in NCBI Build 36, the risk allele and it's frequency, number of cases and controls,
the Odds ratio, and P values. Allelic codes are A = 1, C = 2, G = 3 and T = 4.

| | Pos NCBI | Risk | | No of | No of | | | Seq Id |
|---|---|---|---|---|---|---|---|---|
| Marker | B36 | Allele | Freq. | cases | controls | OR | P-Value | NO |
| rs4803899 | 43419480 | A | 0.386877 | 1776 | 35675 | 1.08816 | 0.0369657 | 405 |
| rs1036233 | 43420054 | A | 0.567307 | 1776 | 35675 | 1.00809 | 0.840867 | 406 |
| rs7246060 | 43423502 | G | 0.577117 | 1776 | 35675 | 1.00789 | 0.843555 | 407 |
| rs12976534 | 43435802 | A | 0.473545 | 1776 | 35675 | 1.07781 | 0.0345114 | 409 |
| rs4803934 | 43438407 | C | 0.464962 | 1776 | 35675 | 1.0791 | 0.0354542 | 410 |
| rs11668070 | 43440753 | G | 0.469429 | 1776 | 35675 | 1.075 | 0.0443733 | 411 |
| rs7250689 | 43445465 | T | 0.470823 | 1776 | 35675 | 1.07073 | 0.0578493 | 412 |
| rs3786872 | 43447929 | G | 0.216697 | 1776 | 35675 | 1.01521 | 0.724665 | 415 |
| rs3786877 | 43451020 | T | 0.572758 | 1776 | 35675 | 1.0161 | 0.650939 | 416 |
| rs8101725 | 43456912 | T | 0.4074 | 1774 | 35629 | 1.00679 | 0.84644 | 418 |
| rs12611009 | 43464321 | C | 0.40738 | 1776 | 35675 | 1.00611 | 0.861486 | 420 |
| rs3826896 | 43465362 | C | 0.407243 | 1763 | 35625 | 1.00376 | 0.914642 | 421 |
| rs3900981 | 43492005 | T | 0.184993 | 1771 | 35571 | 1.05075 | 0.257728 | 430 |
| rs1052375 | 43553173 | G | 0.465617 | 1776 | 35675 | 1.01862 | 0.618262 | 433 |

### Example 3

Further surrogate markers of the anchor markers rs16902094, rs8102476, rs10934853 and rs445114 were identified using results from the 1000 genome project. This project has the goal of finding most genetic variants that have frequencies of at least 1% in the populations studied through sequencing. Details of the project are available on its website http://www.1000genomes.org.

Using data about samples of European origin, SNPs in LD with the anchor markers were identified. These SNPs as tabulated in the Tables 17-20 below represent further surrogates for the anchor markers rs16902094, rs8102476, rs10934853 and rs445114.

TABLE 17
Surrogate markers based on 1000 genome project
(http://www.1000genomes.org) to anchor marker rs10934853 on
Chromosome 3q21.3, with r² > 0.2 in Caucasians. Shown is;
Surrogate marker name, position of surrogate marker in NCBI Build 36,
the allele that is correlated with risk-allele of the anchor marker, and
D′, r², and P-values of the correlation between the markers.
Allelic codes are A = 1, C = 2, G = 3, T = 4.

| | Pos in | Risk | | | | Seq ID |
|---|---|---|---|---|---|---|
| SNP | NCBI B36 | Allele | D′ | r² | p-value | NO: |
| rs16845806 | 129193164 | A | 0.56 | 0.22 | 0.00059 | 21 |
| rs9839080 | 129194687 | T | 0.8 | 0.34 | 4.80E−07 | 500 |
| s.129194713 | 129194713 | T | 1 | 0.26 | 1.30E−07 | 501 |
| s.129195196 | 129195196 | T | 1 | 0.43 | 1.10E−11 | 502 |
| rs7630727 | 129196138 | C | 0.8 | 0.34 | 4.80E−07 | 22 |
| s.129196633 | 129196633 | G | 0.8 | 0.34 | 4.80E−07 | 503 |
| rs1549876 | 129197301 | G | 0.8 | 0.34 | 4.80E−07 | 23 |
| rs6803110 | 129197708 | G | 0.8 | 0.34 | 4.80E−07 | 504 |
| rs1549875 | 129197739 | C | 0.8 | 0.34 | 4.80E−07 | 505 |
| rs1549874 | 129197823 | G | 0.9 | 0.38 | 9.20E−07 | 506 |
| rs17282209 | 129197886 | C | 1 | 0.26 | 1.30E−07 | 24 |
| s.129198601 | 129198601 | A | 0.9 | 0.38 | 9.20E−07 | 507 |
| rs4857832 | 129198952 | A | 0.9 | 0.38 | 9.20E−07 | 508 |
| rs9870753 | 129199275 | G | 0.9 | 0.38 | 9.20E−07 | 509 |
| rs6439104 | 129200392 | C | 0.9 | 0.38 | 9.20E−07 | 25 |
| rs1469660 | 129203417 | C | 0.9 | 0.38 | 9.20E−07 | 510 |
| rs1469659 | 129203430 | T | 0.9 | 0.38 | 9.20E−07 | 26 |
| rs6781473 | 129203731 | T | 0.9 | 0.38 | 9.20E−07 | 511 |
| rs9859280 | 129203932 | T | 0.9 | 0.38 | 9.20E−07 | 512 |
| rs58170120 | 129204023 | A | 0.9 | 0.38 | 9.20E−07 | 513 |
| rs28520291 | 129204065 | C | 1 | 0.36 | 5.50E−10 | 514 |
| rs11924838 | 129204198 | A | 0.9 | 0.38 | 9.20E−07 | 515 |
| rs11917022 | 129204233 | T | 1 | 0.4 | 8.10E−11 | 516 |
| rs11924866 | 129204293 | C | 0.9 | 0.38 | 9.20E−07 | 517 |
| rs10433341 | 129204549 | A | 0.9 | 0.38 | 9.20E−07 | 518 |
| rs7633480 | 129205698 | A | 0.9 | 0.38 | 9.20E−07 | 519 |
| rs7645109 | 129205707 | C | 0.9 | 0.38 | 9.20E−07 | 520 |
| rs7611426 | 129205887 | G | 0.9 | 0.38 | 9.20E−07 | 521 |
| rs7611430 | 129205905 | G | 0.8 | 0.34 | 4.80E−07 | 27 |
| rs67464627 | 129205923 | T | 0.9 | 0.38 | 9.20E−07 | 522 |
| rs6767360 | 129206835 | G | 0.9 | 0.38 | 9.20E−07 | 523 |
| rs6770337 | 129207423 | G | 0.8 | 0.34 | 4.80E−07 | 28 |
| rs6794938 | 129207441 | G | 0.8 | 0.34 | 4.80E−07 | 524 |
| rs6777095 | 129209327 | A | 1 | 0.47 | 1.50E−12 | 29 |
| rs6777197 | 129209427 | A | 1 | 0.47 | 1.50E−12 | 525 |
| rs6777484 | 129209686 | A | 1 | 0.47 | 1.50E−12 | 526 |
| rs6766665 | 129210140 | T | 1 | 0.43 | 1.10E−11 | 527 |
| rs11717102 | 129210442 | C | 0.88 | 0.48 | 1.00E−09 | 528 |
| rs10934838 | 129213009 | C | 0.88 | 0.51 | 3.40E−10 | 529 |
| rs9824657 | 129213456 | T | 1 | 0.47 | 1.50E−12 | 530 |
| rs9809866 | 129214363 | C | 1 | 0.47 | 1.50E−12 | 531 |
| rs4602341 | 129215781 | A | 1 | 0.43 | 1.10E−11 | 30 |
| rs35546672 | 129223143 | C | 0.88 | 0.51 | 3.40E−10 | 532 |
| rs4857833 | 129228387 | G | 0.88 | 0.51 | 3.40E−10 | 31 |
| rs6439108 | 129228455 | G | 0.88 | 0.51 | 3.40E−10 | 32 |
| rs6764517 | 129230531 | A | 1 | 0.47 | 1.50E−12 | 33 |
| rs981447 | 129236378 | G | 0.88 | 0.51 | 3.40E−10 | 34 |
| rs981446 | 129236420 | G | 0.88 | 0.51 | 3.40E−10 | 35 |
| s.129236731 | 129236731 | T | 1 | 0.27 | 1.00E−10 | 533 |
| rs1469658 | 129241904 | T | 1 | 0.47 | 1.50E−12 | 36 |
| rs1469657 | 129242358 | C | 1 | 0.47 | 1.50E−12 | 534 |
| s.129243618 | 129243618 | G | 0.88 | 0.51 | 3.40E−10 | 535 |
| rs4857834 | 129245944 | T | 1 | 0.47 | 1.50E−12 | 536 |
| rs1473246 | 129252098 | G | 0.88 | 0.51 | 3.40E−10 | 537 |
| rs2335772 | 129255226 | G | 0.91 | 0.3 | 1.50E−06 | 37 |
| s.129256165 | 129256165 | A | 0.88 | 0.51 | 3.40E−10 | 538 |
| s.129256166 | 129256166 | G | 1 | 0.26 | 1.90E−10 | 539 |
| rs1030656 | 129256317 | G | 0.88 | 0.51 | 3.40E−10 | 38 |
| rs1030655 | 129256366 | T | 0.88 | 0.51 | 3.40E−10 | 39 |
| rs2335771 | 129262634 | A | 0.88 | 0.51 | 3.40E−10 | 40 |
| rs759945 | 129262772 | G | 1 | 0.47 | 1.50E−12 | 41 |
| s.129264066 | 129264066 | A | 1 | 0.23 | 7.70E−07 | 540 |
| rs4857864 | 129264427 | G | 1 | 0.43 | 1.10E−11 | 541 |
| rs9864797 | 129265117 | G | 1 | 0.47 | 1.50E−12 | 542 |
| rs59766347 | 129265926 | C | 0.88 | 0.51 | 3.40E−10 | 543 |
| rs2075402 | 129266952 | C | 0.88 | 0.51 | 3.40E−10 | 42 |
| rs6439111 | 129267302 | C | 0.88 | 0.51 | 3.40E−10 | 544 |
| rs10934848 | 129273234 | G | 1 | 0.4 | 8.10E−11 | 545 |
| rs2241688 | 129273296 | G | 1 | 0.47 | 1.50E−12 | 546 |
| rs1554534 | 129282359 | G | 1 | 0.43 | 1.10E−11 | 43 |
| rs3732402 | 129288908 | G | 0.88 | 0.51 | 3.40E−10 | 44 |
| rs6439112 | 129289804 | G | 1 | 0.47 | 1.50E−12 | 547 |
| rs7355887 | 129291192 | C | 1 | 0.86 | 9.90E−24 | 548 |
| rs9855015 | 129291505 | A | 1 | 0.47 | 1.50E−12 | 549 |
| rs35724792 | 129292306 | G | 1 | 0.3 | 2.20E−08 | 550 |
| rs34403909 | 129293135 | G | 1 | 0.3 | 2.20E−08 | 551 |
| s.129293428 | 129293428 | G | 1 | 0.3 | 2.20E−08 | 552 |
| rs13091198 | 129294727 | T | 1 | 0.26 | 1.30E−07 | 45 |
| rs55684215 | 129295482 | A | 1 | 0.26 | 1.30E−07 | 553 |
| s.129296501 | 129296501 | T | 1 | 0.3 | 2.20E−08 | 554 |
| rs36069551 | 129296767 | A | 1 | 0.26 | 1.30E−07 | 555 |
| rs11714052 | 129297147 | A | 1 | 0.26 | 1.30E−07 | 46 |
| rs4058156 | 129298165 | T | 1 | 0.47 | 1.50E−12 | 556 |
| rs36059338 | 129298617 | G | 1 | 0.3 | 2.20E−08 | 557 |
| rs34228187 | 129299123 | C | 1 | 0.3 | 2.20E−08 | 558 |
| rs6439113 | 129299262 | A | 1 | 0.47 | 1.50E−12 | 47 |
| s.129299712 | 129299712 | T | 1 | 0.3 | 2.20E−08 | 559 |
| rs6787614 | 129300168 | A | 1 | 0.3 | 2.20E−08 | 48 |
| rs7632756 | 129300198 | C | 1 | 0.86 | 9.90E−24 | 560 |
| rs11720239 | 129300645 | G | 1 | 0.26 | 1.30E−07 | 49 |
| rs34709844 | 129301559 | A | 1 | 0.3 | 2.20E−08 | 561 |
| rs4857866 | 129301752 | G | 1 | 0.86 | 9.90E−24 | 562 |
| rs4857867 | 129301841 | T | 1 | 0.47 | 1.50E−12 | 563 |
| s.129304711 | 129304711 | C | 1 | 0.3 | 2.20E−08 | 564 |
| rs7641133 | 129305010 | T | 1 | 0.86 | 9.90E−24 | 51 |
| s.129306336 | 129306336 | T | 1 | 0.33 | 3.60E−09 | 565 |
| rs9835383 | 129306707 | A | 1 | 0.43 | 1.10E−11 | 566 |
| s.129307349 | 129307349 | G | 1 | 0.3 | 2.20E−08 | 567 |
| s.129307468 | 129307468 | T | 1 | 0.3 | 2.20E−08 | 568 |
| s.129307768 | 129307768 | G | 1 | 0.3 | 2.20E−08 | 569 |
| rs35437840 | 129307996 | T | 1 | 0.3 | 2.20E−08 | 570 |
| s.129309269 | 129309269 | G | 1 | 0.3 | 2.20E−08 | 571 |
| rs11924142 | 129309308 | C | 1 | 0.47 | 1.50E−12 | 52 |
| rs13082038 | 129311146 | G | 1 | 0.26 | 1.30E−07 | 572 |
| rs60387553 | 129311658 | A | 1 | 0.3 | 2.20E−08 | 573 |
| s.129313728 | 129313728 | G | 1 | 0.3 | 2.20E−08 | 574 |
| rs35947214 | 129315921 | G | 1 | 0.3 | 2.20E−08 | 575 |
| rs67940364 | 129316043 | C | 1 | 0.26 | 1.30E−07 | 576 |
| rs7650365 | 129316693 | G | 0.91 | 0.31 | 7.30E−07 | 53 |
| rs6785535 | 129317385 | G | 1 | 0.26 | 1.30E−07 | 577 |
| rs6788879 | 129318319 | A | 1 | 0.47 | 1.50E−12 | 54 |
| rs6439115 | 129318493 | C | 1 | 0.43 | 1.10E−11 | 55 |
| rs4857836 | 129318977 | C | 1 | 0.86 | 9.90E−24 | 56 |
| rs4857837 | 129319009 | A | 1 | 0.86 | 9.90E−24 | 57 |
| s.129320198 | 129320198 | T | 1 | 0.26 | 1.30E−07 | 578 |
| rs11707462 | 129321320 | C | 1 | 0.3 | 2.20E−08 | 58 |
| rs9821568 | 129321689 | G | 1 | 0.47 | 1.50E−12 | 59 |
| rs67577411 | 129322715 | G | 1 | 0.26 | 1.30E−07 | 579 |
| s.129323137 | 129323137 | G | 1 | 0.3 | 2.20E−08 | 580 |
| s.129323200 | 129323200 | A | 1 | 0.3 | 2.20E−08 | 581 |
| s.129323256 | 129323256 | C | 1 | 0.26 | 1.30E−07 | 582 |
| s.129323750 | 129323750 | T | 1 | 0.3 | 2.20E−08 | 583 |
| s.129323788 | 129323788 | A | 1 | 0.26 | 1.30E−07 | 584 |
| s.129324434 | 129324434 | A | 1 | 0.3 | 2.20E−08 | 585 |
| rs6784159 | 129326068 | C | 1 | 0.3 | 2.20E−08 | 60 |
| rs2811475 | 129328391 | C | 1 | 0.47 | 1.50E−12 | 61 |
| s.129329988 | 129329988 | A | 1 | 0.26 | 1.30E−07 | 586 |
| s.129330108 | 129330108 | G | 1 | 0.3 | 2.20E−08 | 587 |
| rs55762931 | 129330365 | T | 1 | 0.3 | 2.20E−08 | 588 |
| rs13095660 | 129330846 | T | 1 | 0.3 | 2.20E−08 | 62 |
| rs6788497 | 129331078 | T | 1 | 0.3 | 2.20E−08 | 589 |
| rs6439116 | 129331757 | C | 1 | 0.3 | 2.20E−08 | 63 |
| rs11714619 | 129332766 | A | 1 | 0.3 | 2.20E−08 | 590 |
| s.129334251 | 129334251 | A | 1 | 0.3 | 2.20E−08 | 591 |
| rs4857868 | 129336163 | T | 1 | 0.47 | 1.50E−12 | 592 |
| rs35347185 | 129337217 | T | 1 | 0.26 | 1.30E−07 | 593 |
| rs2811471 | 129337374 | G | 1 | 0.47 | 1.50E−12 | 594 |
| s.129338521 | 129338521 | G | 1 | 0.23 | 7.70E−07 | 595 |
| s.129338524 | 129338524 | C | 1 | 0.26 | 1.30E−07 | 596 |
| rs13069054 | 129338672 | A | 1 | 0.3 | 2.20E−08 | 597 |
| rs6810006 | 129338748 | A | 1 | 0.3 | 2.20E−08 | 598 |
| s.129339090 | 129339090 | T | 1 | 0.3 | 2.20E−08 | 599 |
| rs6414310 | 129339263 | T | 1 | 0.86 | 9.90E−24 | 64 |
| s.129340623 | 129340623 | C | 1 | 0.3 | 2.20E−08 | 600 |
| rs12496052 | 129341765 | T | 0.87 | 0.42 | 4.10E−08 | 601 |
| rs56175090 | 129342770 | A | 1 | 0.26 | 1.30E−07 | 602 |
| s.129343169 | 129343169 | G | 1 | 0.3 | 2.20E−08 | 603 |
| rs34372443 | 129343184 | G | 1 | 0.3 | 2.20E−08 | 604 |
| s.129343748 | 129343748 | A | 1 | 0.26 | 1.30E−07 | 605 |
| s.129344512 | 129344512 | A | 1 | 0.47 | 1.50E−12 | 606 |
| s.129348249 | 129348249 | G | 1 | 0.3 | 2.20E−08 | 607 |
| rs67451924 | 129350087 | A | 1 | 0.86 | 9.90E−24 | 608 |
| s.129350282 | 129350282 | G | 1 | 0.47 | 1.50E−12 | 609 |
| s.129350965 | 129350965 | A | 1 | 0.3 | 2.20E−08 | 610 |
| s.129351011 | 129351011 | T | 1 | 0.3 | 2.20E−08 | 611 |
| s.129351855 | 129351855 | A | 1 | 0.47 | 1.50E−12 | 612 |
| s.129352517 | 129352517 | C | 0.89 | 0.22 | 2.90E−05 | 613 |
| s.129352750 | 129352750 | A | 1 | 0.86 | 9.90E−24 | 614 |
| s.129352959 | 129352959 | T | 1 | 0.3 | 2.20E−08 | 615 |
| s.129353619 | 129353619 | G | 1 | 0.47 | 1.50E−12 | 616 |
| rs11920225 | 129354865 | G | 1 | 0.47 | 1.50E−12 | 66 |
| rs11711710 | 129355196 | T | 1 | 0.3 | 2.20E−08 | 617 |
| rs2999046 | 129355486 | T | 1 | 0.47 | 1.50E−12 | 618 |
| rs11709066 | 129357602 | A | 1 | 0.3 | 2.20E−08 | 67 |
| rs11716941 | 129358064 | G | 1 | 0.3 | 2.20E−08 | 68 |
| s.129360075 | 129360075 | C | 1 | 0.43 | 1.10E−11 | 619 |
| rs2955114 | 129360446 | C | 1 | 0.47 | 1.50E−12 | 620 |
| s.129360754 | 129360754 | T | 1 | 0.47 | 1.50E−12 | 621 |
| s.129361020 | 129361020 | G | 1 | 0.43 | 1.10E−11 | 622 |
| rs2999048 | 129361106 | A | 1 | 0.86 | 9.90E−24 | 623 |
| s.129361507 | 129361507 | C | 1 | 0.86 | 9.90E−24 | 624 |
| rs2811472 | 129361561 | G | 1 | 0.43 | 1.10E−11 | 69 |
| s.129362916 | 129362916 | C | 1 | 0.26 | 1.30E−07 | 625 |
| s.129364258 | 129364258 | T | 1 | 0.3 | 2.20E−08 | 626 |
| s.129364303 | 129364303 | A | 0.9 | 0.7 | 7.00E−14 | 627 |
| rs2955118 | 129364754 | A | 1 | 0.86 | 9.90E−24 | 628 |
| rs13077913 | 129365294 | A | 1 | 0.3 | 2.20E−08 | 70 |
| rs13077790 | 129365348 | T | 1 | 0.3 | 2.20E−08 | 71 |
| s.129365482 | 129365482 | C | 1 | 0.26 | 1.30E−07 | 629 |
| rs2811473 | 129367627 | C | 1 | 0.47 | 1.50E−12 | 72 |
| rs2687728 | 129367698 | A | 1 | 0.47 | 1.50E−12 | 73 |
| rs10934850 | 129369647 | A | 1 | 0.23 | 7.70E−07 | 74 |
| rs872267 | 129370757 | A | 1 | 0.86 | 9.90E−24 | 75 |
| s.129370865 | 129370865 | G | 1 | 0.26 | 1.30E−07 | 630 |
| rs2955120 | 129371037 | G | 1 | 0.86 | 9.90E−24 | 631 |
| rs2687731 | 129371384 | T | 1 | 0.43 | 1.10E−11 | 76 |
| s.129371668 | 129371668 | T | 1 | 0.47 | 1.50E−12 | 632 |
| rs3122173 | 129371977 | G | 1 | 0.86 | 9.90E−24 | 633 |
| rs3122174 | 129372065 | G | 1 | 0.47 | 1.50E−12 | 77 |
| rs2999051 | 129372094 | C | 0.87 | 0.42 | 4.10E−08 | 78 |
| rs13067650 | 129372232 | A | 1 | 0.3 | 2.20E−08 | 79 |
| rs2248668 | 129373439 | G | 1 | 0.47 | 1.50E−12 | 80 |
| rs2955121 | 129374086 | G | 1 | 0.43 | 1.10E−11 | 81 |
| rs11706455 | 129374681 | A | 1 | 0.3 | 2.20E−08 | 82 |
| rs2999052 | 129374727 | C | 1 | 0.86 | 9.90E−24 | 83 |
| s.129375541 | 129375541 | C | 1 | 0.47 | 1.50E−12 | 634 |
| rs11715394 | 129376254 | T | 1 | 0.3 | 2.20E−08 | 84 |
| s.129376984 | 129376984 | C | 1 | 0.3 | 2.20E−08 | 635 |
| rs2687729 | 129377916 | G | 1 | 0.91 | 2.70E−25 | 85 |
| rs1554535 | 129378676 | A | 1 | 0.86 | 9.90E−24 | 636 |
| s.129380076 | 129380076 | G | 1 | 0.33 | 3.60E−09 | 637 |
| rs2811476 | 129381191 | C | 1 | 0.86 | 9.90E−24 | 638 |
| rs2999053 | 129381223 | A | 1 | 0.47 | 1.50E−12 | 639 |
| rs2811477 | 129381473 | T | 1 | 0.3 | 2.20E−08 | 640 |
| s.129381633 | 129381633 | T | 1 | 0.36 | 5.50E−10 | 641 |
| rs2811478 | 129382314 | T | 1 | 0.3 | 2.20E−08 | 86 |
| s.129382676 | 129382676 | A | 1 | 0.47 | 1.50E−12 | 642 |
| rs2999060 | 129383184 | G | 1 | 0.47 | 1.50E−12 | 87 |
| rs2999055 | 129383212 | T | 1 | 0.47 | 1.50E−12 | 643 |
| s.129383929 | 129383929 | C | 1 | 0.2 | 4.30E−06 | 644 |
| s.129384476 | 129384476 | A | 1 | 0.86 | 9.90E−24 | 645 |
| rs2811480 | 129384781 | T | 1 | 0.86 | 9.90E−24 | 646 |
| rs2999056 | 129386212 | A | 1 | 0.43 | 1.10E−11 | 88 |
| rs2955123 | 129386368 | C | 1 | 0.47 | 1.50E−12 | 89 |
| s.129386658 | 129386658 | G | 1 | 0.2 | 4.30E−06 | 647 |
| rs2811517 | 129386880 | C | 1 | 0.47 | 1.50E−12 | 90 |
| s.129387186 | 129387186 | C | 1 | 0.86 | 9.90E−24 | 648 |
| rs2811482 | 129387633 | T | 1 | 0.86 | 9.90E−24 | 649 |
| rs2811516 | 129388141 | G | 1 | 0.86 | 9.90E−24 | 91 |
| rs2811515 | 129388208 | A | 1 | 0.86 | 9.90E−24 | 92 |
| s.129388338 | 129388338 | T | 1 | 0.47 | 1.50E−12 | 650 |
| rs2811514 | 129390619 | G | 1 | 0.86 | 9.90E−24 | 93 |
| rs2811513 | 129392254 | A | 1 | 0.86 | 9.90E−24 | 651 |
| s.129392448 | 129392448 | T | 1 | 0.43 | 1.10E−11 | 652 |
| rs2811512 | 129394510 | A | 1 | 0.86 | 9.90E−24 | 94 |
| s.129395583 | 129395583 | A | 1 | 0.26 | 1.30E−07 | 653 |
| rs2811511 | 129395678 | A | 1 | 0.3 | 2.20E−08 | 95 |
| rs883238 | 129395953 | G | 1 | 0.86 | 9.90E−24 | 96 |
| rs940061 | 129396404 | G | 1 | 0.4 | 8.10E−11 | 97 |
| s.129396518 | 129396518 | A | 1 | 0.47 | 1.50E−12 | 654 |
| rs2811510 | 129397202 | G | 1 | 0.47 | 1.50E−12 | 98 |
| rs2811483 | 129397363 | C | 1 | 0.47 | 1.50E−12 | 99 |
| rs2811484 | 129397543 | A | 1 | 0.47 | 1.50E−12 | 100 |
| rs2687730 | 129398147 | T | 1 | 0.3 | 2.20E−08 | 101 |
| rs2955124 | 129399180 | T | 1 | 0.47 | 1.50E−12 | 655 |
| rs2811509 | 129399241 | A | 1 | 0.86 | 9.90E−24 | 102 |
| rs2492285 | 129400690 | A | 1 | 0.86 | 9.90E−24 | 103 |
| rs2687720 | 129401645 | T | 1 | 0.86 | 9.90E−24 | 104 |
| s.129401735 | 129401735 | A | 1 | 0.86 | 9.90E−24 | 656 |
| s.129401736 | 129401736 | T | 1 | 0.86 | 9.90E−24 | 657 |
| s.129402107 | 129402107 | T | 1 | 0.47 | 1.50E−12 | 658 |
| rs2811508 | 129402713 | C | 1 | 0.86 | 9.90E−24 | 105 |
| rs2811486 | 129402765 | G | 1 | 0.3 | 2.20E−08 | 106 |
| rs6439119 | 129405877 | T | 1 | 0.86 | 9.90E−24 | 107 |
| s.129406500 | 129406500 | A | 1 | 0.47 | 1.50E−12 | 659 |
| rs2955125 | 129407882 | G | 1 | 0.47 | 1.50E−12 | 108 |
| s.129408735 | 129408735 | C | 0.87 | 0.42 | 4.10E−08 | 660 |
| rs2955126 | 129408944 | A | 1 | 0.3 | 2.20E−08 | 109 |
| rs2955127 | 129409206 | G | 1 | 0.47 | 1.50E−12 | 110 |
| s.129410967 | 129410967 | T | 1 | 0.47 | 1.50E−12 | 661 |
| rs2955129 | 129411897 | T | 1 | 0.3 | 2.20E−08 | 112 |
| rs9838120 | 129413149 | A | 1 | 0.86 | 9.90E−24 | 662 |
| rs7374072 | 129413556 | C | 1 | 0.86 | 9.90E−24 | 113 |
| rs2999090 | 129414030 | G | 1 | 0.3 | 2.20E−08 | 114 |
| rs7372439 | 129414092 | A | 1 | 0.86 | 9.90E−24 | 115 |
| s.129415893 | 129415893 | G | 1 | 0.86 | 9.90E−24 | 663 |
| rs4857871 | 129416476 | C | 1 | 0.86 | 9.90E−24 | 116 |
| rs4857872 | 129416512 | T | 1 | 0.86 | 9.90E−24 | 117 |
| rs4857873 | 129416908 | A | 1 | 0.86 | 9.90E−24 | 118 |
| rs4857874 | 129416959 | A | 1 | 0.86 | 9.90E−24 | 664 |
| rs4857875 | 129416966 | C | 1 | 0.86 | 9.90E−24 | 665 |
| rs6770140 | 129417019 | T | 1 | 0.86 | 9.90E−24 | 119 |
| rs4384971 | 129417464 | C | 1 | 0.86 | 9.90E−24 | 120 |
| rs2999089 | 129417849 | C | 1 | 0.3 | 2.20E−08 | 121 |
| rs6439121 | 129418151 | G | 1 | 0.86 | 9.90E−24 | 122 |
| rs9879865 | 129419217 | C | 1 | 0.86 | 9.90E−24 | 666 |
| rs9879866 | 129419222 | C | 1 | 0.82 | 2.60E−22 | 667 |
| rs2254379 | 129420016 | C | 1 | 0.47 | 1.50E−12 | 123 |
| rs9847576 | 129420335 | A | 1 | 0.86 | 9.90E−24 | 668 |
| rs2955130 | 129420504 | G | 1 | 0.3 | 2.20E−08 | 124 |
| rs9814834 | 129420827 | G | 1 | 0.86 | 9.90E−24 | 125 |
| rs2811549 | 129421398 | T | 1 | 0.3 | 2.20E−08 | 669 |
| rs9857235 | 129421724 | A | 1 | 0.86 | 9.90E−24 | 670 |
| s.129422134 | 129422134 | A | 1 | 0.47 | 1.50E−12 | 671 |
| rs2955132 | 129422916 | C | 1 | 0.47 | 1.50E−12 | 126 |
| rs9845651 | 129423043 | T | 1 | 0.86 | 9.90E−24 | 127 |
| rs7372902 | 129423130 | G | 1 | 0.86 | 9.90E−24 | 672 |
| rs6439122 | 129423224 | A | 1 | 0.86 | 9.90E−24 | 128 |
| s.129423563 | 129423563 | T | 0.9 | 0.28 | 5.50E−06 | 673 |
| s.129423762 | 129423762 | T | 1 | 0.47 | 1.50E−12 | 674 |
| rs6439123 | 129424129 | T | 1 | 0.86 | 9.90E−24 | 675 |
| s.129424637 | 129424637 | G | 1 | 0.47 | 1.50E−12 | 676 |
| rs9873786 | 129425069 | A | 1 | 0.86 | 9.90E−24 | 129 |
| rs4857838 | 129426018 | A | 1 | 0.86 | 9.90E−24 | 130 |
| rs6775988 | 129427167 | C | 1 | 0.86 | 9.90E−24 | 131 |
| rs2955133 | 129428012 | T | 1 | 0.26 | 1.30E−07 | 677 |
| rs6777054 | 129428039 | G | 1 | 0.86 | 9.90E−24 | 678 |
| rs9830294 | 129429478 | G | 1 | 0.86 | 9.90E−24 | 132 |
| s.129429722 | 129429722 | C | 1 | 0.43 | 1.10E−11 | 679 |
| rs4857877 | 129430750 | A | 1 | 0.86 | 9.90E−24 | 133 |
| rs4857878 | 129430839 | T | 1 | 0.86 | 9.90E−24 | 680 |
| s.129431649 | 129431649 | C | 1 | 0.86 | 9.90E−24 | 681 |
| rs2999086 | 129433938 | C | 1 | 0.3 | 2.20E−08 | 134 |
| rs2999085 | 129434249 | G | 1 | 0.3 | 2.20E−08 | 135 |
| rs2999084 | 129435178 | A | 1 | 0.3 | 2.20E−08 | 136 |
| rs6809833 | 129435609 | A | 1 | 0.26 | 1.30E−07 | 682 |
| s.129436659 | 129436659 | G | 0.91 | 0.29 | 2.90E−06 | 683 |
| rs2999083 | 129437124 | C | 1 | 0.3 | 2.20E−08 | 137 |
| rs2999082 | 129438878 | A | 1 | 0.3 | 2.20E−08 | 684 |
| rs2999081 | 129438962 | A | 1 | 0.3 | 2.20E−08 | 138 |
| rs2999080 | 129439398 | G | 1 | 0.3 | 2.20E−08 | 685 |
| rs2999079 | 129439922 | T | 1 | 0.3 | 2.20E−08 | 139 |
| rs4074440 | 129440703 | G | 1 | 0.86 | 9.90E−24 | 140 |
| rs2999076 | 129441382 | T | 1 | 0.3 | 2.20E−08 | 686 |
| rs2955077 | 129441559 | A | 1 | 0.3 | 2.20E−08 | 141 |
| rs2955078 | 129441876 | A | 1 | 0.3 | 2.20E−08 | 687 |
| rs2955079 | 129442514 | T | 1 | 0.3 | 2.20E−08 | 688 |
| rs2955080 | 129442555 | C | 1 | 0.26 | 1.30E−07 | 689 |
| s.129442734 | 129442734 | T | 1 | 0.43 | 1.10E−11 | 690 |
| rs2999074 | 129442899 | T | 1 | 0.3 | 2.20E−08 | 691 |
| rs2955081 | 129442989 | A | 1 | 0.3 | 2.20E−08 | 692 |
| rs2955082 | 129443024 | G | 1 | 0.3 | 2.20E−08 | 693 |
| rs2955083 | 129443868 | T | 1 | 0.3 | 2.20E−08 | 694 |
| rs2955084 | 129443995 | T | 1 | 0.3 | 2.20E−08 | 695 |
| rs9843281 | 129444086 | A | 1 | 0.86 | 9.90E−24 | 142 |
| rs2999073 | 129445019 | G | 1 | 0.3 | 2.20E−08 | 143 |
| rs2955085 | 129445447 | G | 1 | 0.3 | 2.20E−08 | 144 |
| rs2999072 | 129445566 | G | 1 | 0.3 | 2.20E−08 | 145 |
| s.129445881 | 129445881 | G | 1 | 0.3 | 2.20E−08 | 696 |
| s.129445882 | 129445882 | A | 1 | 0.3 | 2.20E−08 | 697 |
| rs13434079 | 129446138 | A | 1 | 0.86 | 9.90E−24 | 146 |
| rs2999071 | 129446305 | G | 1 | 0.3 | 2.20E−08 | 698 |
| rs2955088 | 129446400 | C | 1 | 0.3 | 2.20E−08 | 147 |
| rs2999070 | 129447341 | G | 1 | 0.3 | 2.20E−08 | 148 |
| rs17343355 | 129449481 | C | 0.91 | 0.29 | 2.90E−06 | 149 |
| rs2955089 | 129452020 | G | 1 | 0.3 | 2.20E−08 | 699 |
| rs2955090 | 129452061 | T | 1 | 0.3 | 2.20E−08 | 150 |
| rs2955091 | 129454051 | G | 1 | 0.26 | 1.30E−07 | 151 |
| rs2999069 | 129454299 | C | 1 | 0.3 | 2.20E−08 | 152 |
| rs2955092 | 129455218 | C | 1 | 0.3 | 2.20E−08 | 153 |
| rs2955094 | 129459613 | A | 1 | 0.3 | 2.20E−08 | 154 |
| rs2955095 | 129460355 | A | 1 | 0.3 | 2.20E−08 | 155 |
| rs2955096 | 129460556 | A | 1 | 0.3 | 2.20E−08 | 156 |
| rs2999068 | 129460668 | T | 1 | 0.3 | 2.20E−08 | 157 |
| rs2955097 | 129461195 | G | 1 | 0.3 | 2.20E−08 | 700 |
| rs2999067 | 129461221 | C | 1 | 0.3 | 2.20E−08 | 158 |
| s.129461485 | 129461485 | G | 1 | 0.86 | 9.90E−24 | 701 |
| rs2955098 | 129462191 | T | 1 | 0.3 | 2.20E−08 | 702 |
| rs2955099 | 129462344 | G | 1 | 0.26 | 1.30E−07 | 159 |
| s.129462511 | 129462511 | A | 1 | 0.2 | 9.80E−09 | 703 |
| rs2999066 | 129462976 | T | 1 | 0.3 | 2.20E−08 | 160 |
| rs2999065 | 129464128 | A | 1 | 0.3 | 2.20E−08 | 161 |
| rs2999035 | 129465899 | T | 1 | 0.47 | 1.50E−12 | 163 |
| rs2811544 | 129466185 | T | 1 | 0.3 | 2.20E−08 | 164 |
| rs2811543 | 129466486 | T | 1 | 0.3 | 2.20E−08 | 165 |
| rs2811542 | 129466490 | A | 1 | 0.3 | 2.20E−08 | 704 |
| s.129466683 | 129466683 | A | 1 | 0.3 | 2.20E−08 | 705 |
| s.129466684 | 129466684 | C | 1 | 0.3 | 2.20E−08 | 706 |
| s.129466686 | 129466686 | G | 1 | 0.36 | 5.50E−10 | 707 |
| rs2811541 | 129467484 | C | 1 | 0.26 | 1.30E−07 | 166 |
| rs2811540 | 129468259 | T | 1 | 0.3 | 2.20E−08 | 167 |
| rs2811539 | 129469163 | C | 1 | 0.3 | 2.20E−08 | 168 |
| rs2811538 | 129469321 | A | 1 | 0.26 | 1.30E−07 | 169 |
| rs2811396 | 129470046 | A | 1 | 0.3 | 2.20E−08 | 170 |
| rs2811400 | 129470557 | G | 1 | 0.26 | 1.30E−07 | 171 |
| rs2811537 | 129470614 | A | 1 | 0.3 | 2.20E−08 | 172 |
| s.129470765 | 129470765 | T | 0.91 | 0.29 | 2.90E−06 | 708 |
| rs2999064 | 129470930 | G | 1 | 0.47 | 1.50E−12 | 173 |
| rs2811536 | 129471609 | A | 1 | 0.3 | 2.20E−08 | 174 |
| rs2811534 | 129473798 | A | 1 | 0.3 | 2.20E−08 | 175 |
| rs2811413 | 129473839 | C | 0.9 | 0.7 | 7.00E−14 | 176 |
| rs2811414 | 129473867 | G | 1 | 0.26 | 1.30E−07 | 709 |
| rs2811533 | 129474419 | G | 1 | 0.26 | 1.30E−07 | 178 |
| rs2811416 | 129474628 | T | 1 | 0.3 | 2.20E−08 | 179 |
| rs2811532 | 129474860 | G | 1 | 0.26 | 1.30E−07 | 180 |
| rs2811531 | 129475185 | G | 1 | 0.3 | 2.20E−08 | 181 |
| rs2811530 | 129475856 | A | 1 | 0.3 | 2.20E−08 | 710 |
| rs2955100 | 129476015 | G | 1 | 0.3 | 2.20E−08 | 182 |
| s.129476296 | 129476296 | A | 1 | 0.3 | 2.20E−08 | 711 |
| rs2999061 | 129476313 | A | 1 | 0.3 | 2.20E−08 | 183 |
| rs2811529 | 129476850 | G | 1 | 0.86 | 9.90E−24 | 184 |
| rs2811528 | 129477126 | A | 1 | 0.3 | 2.20E−08 | 712 |
| rs2811527 | 129477294 | A | 1 | 0.3 | 2.20E−08 | 185 |
| rs2811526 | 129478408 | A | 1 | 0.3 | 2.20E−08 | 713 |
| rs2811373 | 129480119 | T | 1 | 0.3 | 2.20E−08 | 186 |
| rs2811381 | 129480602 | A | 1 | 0.3 | 2.20E−08 | 714 |
| rs2811525 | 129482120 | T | 1 | 0.3 | 2.20E−08 | 187 |
| rs2811524 | 129482645 | G | 1 | 0.3 | 2.20E−08 | 715 |
| rs2811386 | 129482705 | A | 1 | 0.3 | 2.20E−08 | 716 |
| rs7374952 | 129484057 | T | 1 | 0.3 | 2.20E−08 | 188 |
| rs7374227 | 129484205 | G | 1 | 0.3 | 2.20E−08 | 189 |
| rs6765233 | 129485540 | T | 1 | 0.82 | 2.60E−22 | 717 |
| rs4593050 | 129487221 | T | 1 | 0.3 | 2.20E−08 | 190 |
| rs6439124 | 129490156 | A | 1 | 0.3 | 2.20E−08 | 191 |
| rs7373998 | 129490913 | A | 1 | 0.3 | 2.20E−08 | 192 |
| rs2955101 | 129492302 | A | 1 | 0.3 | 2.20E−08 | 193 |
| rs2811523 | 129494087 | A | 1 | 0.26 | 1.30E−07 | 718 |
| rs2811522 | 129494113 | T | 1 | 0.3 | 2.20E−08 | 719 |
| rs2811520 | 129494967 | G | 1 | 0.3 | 2.20E−08 | 720 |
| rs11718884 | 129495205 | A | 1 | 0.2 | 9.80E−09 | 721 |
| rs10934852 | 129495212 | A | 0.91 | 0.29 | 2.90E−06 | 722 |
| rs2811519 | 129495566 | T | 1 | 0.3 | 2.20E−08 | 194 |
| rs2811518 | 129496335 | G | 1 | 0.3 | 2.20E−08 | 195 |
| rs2811387 | 129497868 | C | 1 | 0.3 | 2.20E−08 | 723 |
| rs2955103 | 129497926 | C | 0.87 | 0.42 | 4.10E−08 | 196 |
| rs2811388 | 129501137 | A | 1 | 0.3 | 2.20E−08 | 197 |
| rs2999036 | 129503391 | G | 1 | 0.3 | 2.20E−08 | 198 |
| s.129504052 | 129504052 | A | 1 | 0.26 | 1.30E−07 | 724 |
| rs2811390 | 129504171 | T | 1 | 0.3 | 2.20E−08 | 199 |
| rs2811391 | 129505058 | A | 1 | 0.3 | 2.20E−08 | 200 |
| rs2811392 | 129506666 | G | 1 | 0.3 | 2.20E−08 | 725 |
| rs2811393 | 129506689 | T | 1 | 0.3 | 2.20E−08 | 201 |
| rs2037965 | 129507734 | C | 1 | 0.26 | 1.30E−07 | 202 |
| rs2811397 | 129509927 | C | 1 | 0.26 | 1.30E−07 | 203 |
| rs6805582 | 129511698 | A | 1 | 0.3 | 2.20E−08 | 204 |
| rs6805621 | 129511894 | T | 1 | 0.3 | 2.20E−08 | 205 |
| s.129513033 | 129513033 | A | 1 | 0.3 | 2.20E−08 | 726 |
| s.129513164 | 129513164 | T | 1 | 0.3 | 2.20E−08 | 727 |
| rs6794591 | 129513909 | T | 1 | 0.3 | 2.20E−08 | 206 |
| rs16843876 | 129515230 | G | 1 | 0.26 | 1.30E−07 | 207 |
| rs11706826 | 129515681 | T | 1 | 0.3 | 2.20E−08 | 209 |
| rs11706908 | 129515738 | A | 1 | 0.26 | 1.30E−07 | 210 |
| rs6771646 | 129517225 | G | 1 | 0.3 | 2.20E−08 | 211 |
| rs13095166 | 129519476 | T | 1 | 0.58 | 2.10E−15 | 212 |
| rs10934853 | 129521063 | A | 1 | 1 | | 1 |
| rs12486127 | 129521379 | A | 1 | 0.58 | 2.10E−15 | 214 |
| rs12486156 | 129521524 | T | 1 | 0.43 | 1.10E−11 | 215 |
| s.129524520 | 129524520 | G | 1 | 0.3 | 2.20E−08 | 728 |
| rs55925538 | 129525567 | A | 1 | 0.3 | 2.20E−08 | 729 |
| rs55989768 | 129525694 | C | 1 | 0.3 | 2.20E−08 | 730 |
| rs55675294 | 129525740 | A | 1 | 0.3 | 2.20E−08 | 731 |
| s.129526610 | 129526610 | T | 1 | 0.26 | 1.30E−07 | 732 |
| rs6772407 | 129527062 | T | 1 | 0.3 | 2.20E−08 | 217 |
| s.129529162 | 129529162 | A | 1 | 0.2 | 4.30E−06 | 733 |
| rs4857841 | 129529333 | A | 1 | 1 | 1.80E−29 | 218 |
| s.129529699 | 129529699 | C | 1 | 0.3 | 2.20E−08 | 734 |
| rs11710704 | 129529926 | A | 1 | 0.3 | 2.20E−08 | 219 |
| s.129531514 | 129531514 | T | 1 | 0.2 | 4.30E−06 | 735 |
| rs11706304 | 129533304 | G | 1 | 0.3 | 2.20E−08 | 736 |
| rs16844002 | 129536177 | T | 1 | 0.3 | 2.20E−08 | 220 |
| rs6798749 | 129539587 | A | 1 | 0.3 | 2.20E−08 | 221 |
| s.129541307 | 129541307 | T | 1 | 1 | 1.80E−29 | 737 |
| s.129541983 | 129541983 | C | 1 | 0.3 | 2.20E−08 | 738 |
| rs1735558 | 129542300 | A | 1 | 0.54 | 2.00E−14 | 222 |
| rs58986862 | 129544379 | T | 1 | 0.3 | 2.20E−08 | 739 |
| rs56850662 | 129544612 | T | 1 | 0.3 | 2.20E−08 | 740 |
| rs60399786 | 129544760 | A | 1 | 0.3 | 2.20E−08 | 741 |
| rs4857879 | 129546808 | C | 1 | 0.58 | 2.10E−15 | 223 |
| s.129548003 | 129548003 | T | 1 | 0.3 | 2.20E−08 | 742 |
| s.129548156 | 129548156 | T | 1 | 0.3 | 2.20E−08 | 743 |
| s.129548505 | 129548505 | C | 1 | 0.3 | 2.20E−08 | 744 |
| s.129549804 | 129549804 | T | 1 | 0.33 | 3.60E−09 | 745 |
| rs11709611 | 129549965 | C | 1 | 0.3 | 2.20E−08 | 746 |
| rs11721213 | 129550131 | T | 1 | 0.3 | 2.20E−08 | 224 |
| s.129552202 | 129552202 | G | 1 | 0.2 | 4.30E−06 | 747 |
| rs1735549 | 129554499 | T | 1 | 0.86 | 9.90E−24 | 225 |
| rs11711096 | 129555192 | A | 1 | 0.43 | 1.10E−11 | 748 |
| rs1735546 | 129558088 | G | 1 | 0.86 | 9.90E−24 | 226 |
| rs12632366 | 129560248 | G | 1 | 0.26 | 1.90E−10 | 227 |
| rs6785384 | 129560292 | T | 1 | 0.86 | 9.90E−24 | 749 |
| rs1735545 | 129563950 | C | 1 | 0.82 | 2.60E−22 | 228 |
| rs1702122 | 129566022 | G | 1 | 0.78 | 5.40E−21 | 229 |
| rs1108313 | 129567780 | G | 1 | 0.29 | 3.00E−11 | 230 |
| rs1735538 | 129574792 | G | 0.89 | 0.73 | 1.70E−13 | 231 |
| rs1702119 | 129577183 | C | 1 | 0.78 | 5.40E−21 | 232 |
| rs1702118 | 129577968 | G | 1 | 0.47 | 1.50E−12 | 233 |
| rs3021461 | 129578342 | C | 1 | 0.78 | 5.40E−21 | 234 |
| rs2977565 | 129578457 | A | 1 | 0.73 | 9.40E−20 | 235 |
| rs2293947 | 129580186 | C | 1 | 0.23 | 7.70E−07 | 236 |
| rs2977562 | 129588957 | G | 0.8 | 0.64 | 1.30E−11 | 750 |
| rs7373685 | 129589710 | C | 0.8 | 0.64 | 1.30E−11 | 751 |
| rs1625296 | 129590672 | A | 0.84 | 0.64 | 1.60E−11 | 752 |
| rs741925 | 129592606 | T | 1 | 0.47 | 1.50E−12 | 237 |
| rs3887841 | 129592631 | C | 0.79 | 0.6 | 5.50E−11 | 753 |
| s.129593080 | 129593080 | C | 1 | 0.47 | 1.50E−12 | 754 |
| rs729847 | 129593460 | A | 0.8 | 0.61 | 4.00E−11 | 238 |
| rs4241495 | 129593864 | C | 0.8 | 0.61 | 4.00E−11 | 755 |
| rs1620440 | 129594997 | C | 1 | 0.47 | 1.50E−12 | 240 |
| s.129596519 | 129596519 | C | 1 | 0.23 | 1.90E−09 | 756 |
| rs7632169 | 129597277 | C | 0.8 | 0.61 | 4.00E−11 | 241 |
| rs1735527 | 129598071 | G | 1 | 0.43 | 1.10E−11 | 242 |
| s.129600263 | 129600263 | A | 1 | 0.4 | 8.10E−11 | 757 |
| rs760383 | 129602255 | G | 0.78 | 0.56 | 3.80E−10 | 243 |
| rs2999031 | 129604192 | T | 1 | 0.26 | 1.90E−10 | 246 |
| rs2659685 | 129605086 | A | 0.75 | 0.56 | 3.10E−10 | 248 |
| s.129605225 | 129605225 | C | 1 | 0.2 | 4.30E−06 | 758 |
| rs1735537 | 129605510 | C | 0.75 | 0.57 | 1.60E−10 | 250 |
| rs2977564 | 129606476 | G | 1 | 0.22 | 3.40E−09 | 252 |
| rs60672471 | 129608408 | C | 1 | 0.26 | 1.30E−07 | 759 |
| s.129617004 | 129617004 | T | 1 | 0.23 | 7.70E−07 | 760 |
| s.129631956 | 129631956 | G | 1 | 0.26 | 1.30E−07 | 761 |
| rs2969249 | 129643597 | A | 1 | 0.54 | 2.00E−14 | 762 |
| s.129647692 | 129647692 | T | 1 | 0.36 | 5.50E−10 | 763 |

TABLE 18
Surrogate markers based on 1000 genome project
(http://www.1000genomes.org) to anchor marker rs16902094 on
Chromosome 8q24.2, with r² > 0.2 in Caucasians.
Shown is; Surrogate marker name, position of surrogate marker in
NCBI Build 36, the allele that is correlated with risk-allele of the
anchor marker, and D′, r², and P-values of the correlation
between the markers. Allelic codes are A = 1, C = 2, G = 3, T = 4.

| | Pos in | Risk | | | | Seq ID |
|---|---|---|---|---|---|---|
| SNP | NCBI B36 | Allele | D′ | r² | p-value | NO: |
| rs283716 | 128374349 | A | 0.69 | 0.48 | 1.20E−07 | 438 |
| rs13251915 | 128377137 | T | 0.7 | 0.29 | 3.10E−05 | 271 |
| s.128389528 | 128389528 | G | 1 | 1 | 1.70E−25 | 439 |
| s.128390158 | 128390158 | G | 1 | 1 | 1.70E−25 | 440 |
| s.128390595 | 128390595 | C | 1 | 1 | 1.70E−25 | 441 |
| s.128390665 | 128390665 | C | 1 | 1 | 1.70E−25 | 442 |
| s.128390765 | 128390765 | C | 1 | 1 | 1.70E−25 | 443 |
| s.128390866 | 128390866 | T | 1 | 1 | 1.70E−25 | 444 |
| s.128392339 | 128392339 | A | 1 | 1 | 1.70E−25 | 445 |
| s.128392802 | 128392802 | T | 1 | 0.25 | 2.90E−06 | 446 |
| s.128392906 | 128392906 | T | 1 | 1 | 1.70E−25 | 447 |
| s.128392913 | 128392913 | G | 1 | 1 | 1.70E−25 | 448 |
| s.128393073 | 128393073 | C | 1 | 0.78 | 3.50E−18 | 449 |
| s.128399692 | 128399692 | G | 1 | 0.78 | 3.50E−18 | 450 |
| s.128399737 | 128399737 | C | 1 | 0.89 | 3.00E−21 | 451 |
| s.128401223 | 128401223 | A | 1 | 0.2 | 3.60E−08 | 452 |
| s.128401569 | 128401569 | G | 1 | 1 | 1.70E−25 | 453 |
| s.128402402 | 128402402 | C | 1 | 1 | 1.70E−25 | 454 |
| s.128402747 | 128402747 | A | 1 | 1 | 1.70E−25 | 455 |
| s.128403334 | 128403334 | C | 1 | 0.2 | 3.60E−08 | 456 |
| s.128404174 | 128404174 | A | 1 | 0.2 | 3.60E−08 | 457 |
| s.128404261 | 128404261 | G | 1 | 1 | 1.70E−25 | 458 |
| s.128404718 | 128404718 | A | 1 | 1 | 1.70E−25 | 459 |
| s.128405184 | 128405184 | G | 1 | 0.38 | 4.70E−09 | 460 |
| s.128405235 | 128405235 | G | 1 | 1 | 1.70E−25 | 461 |
| s.128405846 | 128405846 | C | 1 | 1 | 1.70E−25 | 462 |
| s.128406461 | 128406461 | A | 1 | 0.76 | 6.50E−20 | 463 |
| s.128407440 | 128407440 | A | 1 | 1 | 1.70E−25 | 464 |
| s.128409306 | 128409306 | G | 1 | 1 | 1.70E−25 | 465 |
| s.128409403 | 128409403 | A | 1 | 1 | 1.70E−25 | 466 |
| rs16902103 | 128409556 | C | 1 | 1 | 1.70E−25 | 286 |
| s.128409719 | 128409719 | T | 1 | 1 | 1.70E−25 | 467 |
| s.128409736 | 128409736 | T | 1 | 1 | 1.70E−25 | 468 |
| rs16902104 | 128410090 | T | 1 | 1 | | 287 |
| s.128410992 | 128410992 | C | 1 | 0.89 | 3.00E−21 | 469 |
| s.128411248 | 128411248 | A | 0.87 | 0.36 | 7.80E−06 | 470 |
| s.128411269 | 128411269 | C | 1 | 0.38 | 4.70E−09 | 471 |
| s.128411808 | 128411808 | T | 0.94 | 0.83 | 2.90E−15 | 472 |
| rs73336742 | 128417585 | T | 0.86 | 0.61 | 3.70E−10 | 473 |
| rs16902118 | 128417799 | G | 0.86 | 0.61 | 3.70E−10 | 292 |
| s.128423946 | 128423946 | T | 1 | 0.29 | 3.60E−07 | 474 |
| rs16902121 | 128424100 | A | 0.84 | 0.51 | 2.10E−08 | 294 |
| rs73336758 | 128424416 | T | 0.85 | 0.56 | 3.00E−09 | 475 |
| rs59561127 | 128424749 | A | 0.85 | 0.56 | 3.00E−09 | 476 |
| rs73336767 | 128428048 | G | 0.85 | 0.56 | 3.00E−09 | 477 |
| rs11785277 | 128434265 | C | 0.85 | 0.56 | 3.00E−09 | 296 |
| rs11774777 | 128434381 | C | 0.85 | 0.56 | 3.00E−09 | 478 |
| s.128434384 | 128434384 | A | 0.85 | 0.56 | 3.00E−09 | 479 |
| rs11774827 | 128434523 | C | 0.84 | 0.51 | 2.10E−08 | 297 |
| rs11781774 | 128434880 | T | 0.85 | 0.56 | 3.00E−09 | 480 |
| rs35850773 | 128435129 | C | 0.85 | 0.56 | 3.00E−09 | 481 |
| rs13267256 | 128435219 | T | 0.85 | 0.56 | 3.00E−09 | 482 |
| rs13266502 | 128435227 | G | 0.85 | 0.56 | 3.00E−09 | 483 |
| s.128435347 | 128435347 | G | 0.84 | 0.51 | 2.10E−08 | 484 |
| s.128435349 | 128435349 | A | 0.84 | 0.51 | 2.10E−08 | 485 |
| rs11782693 | 128435626 | G | 0.85 | 0.56 | 3.00E−09 | 298 |
| rs11782700 | 128435678 | T | 0.84 | 0.51 | 2.10E−08 | 299 |
| rs11782735 | 128435786 | T | 0.85 | 0.56 | 3.00E−09 | 300 |
| s.128435936 | 128435936 | G | 0.85 | 0.56 | 3.00E−09 | 486 |
| rs11783559 | 128436107 | T | 0.85 | 0.56 | 3.00E−09 | 301 |
| rs11783615 | 128436189 | A | 0.85 | 0.56 | 3.00E−09 | 302 |
| rs73336790 | 128436288 | G | 0.84 | 0.51 | 2.10E−08 | 487 |
| s.128436330 | 128436330 | C | 0.76 | 0.27 | 5.10E−05 | 488 |
| rs36072021 | 128436877 | A | 0.85 | 0.56 | 3.00E−09 | 489 |
| s.128438115 | 128438115 | A | 0.85 | 0.56 | 3.00E−09 | 490 |
| rs55885383 | 128438925 | A | 0.85 | 0.56 | 3.00E−09 | 491 |
| s.128439370 | 128439370 | T | 0.85 | 0.56 | 3.00E−09 | 492 |
| s.128439371 | 128439371 | T | 0.85 | 0.56 | 3.00E−09 | 493 |
| rs56001747 | 128447992 | G | 0.85 | 0.56 | 3.00E−09 | 494 |
| rs11784125 | 128449102 | G | 0.84 | 0.51 | 2.10E−08 | 303 |
| rs11776260 | 128451670 | G | 0.78 | 0.51 | 2.00E−08 | 304 |
| rs11774907 | 128453272 | C | 0.78 | 0.51 | 2.00E−08 | 305 |
| rs16902127 | 128453599 | A | 0.78 | 0.51 | 2.00E−08 | 306 |
| s.128454871 | 128454871 | A | 0.78 | 0.51 | 2.00E−08 | 495 |
| s.128459146 | 128459146 | T | 0.79 | 0.36 | 3.10E−06 | 496 |
| rs731900 | 128459842 | A | 0.6 | 0.31 | 2.80E−05 | 308 |
| rs6982138 | 128464850 | G | 0.52 | 0.21 | 0.00029 | 497 |
| s.128468260 | 128468260 | A | 1 | 0.25 | 2.90E−06 | 498 |
| s.128468265 | 128468265 | G | 0.69 | 0.27 | 0.00011 | 499 |

TABLE 19
Surrogate markers based on 1000 genome project
(http://www.1000genomes.org) to anchor marker rs445114 on
Chromosome 8q24.21, with r² > 0.2 in Caucasians.
Shown is; Surrogate marker name, position of surrogate marker
in NCBI Build 36, the allele that is correlated with risk-allele of the
anchor marker, and D′, r², and P-values of the correlation
between the markers. Allelic codes are A = 1, C = 2, G = 3, T = 4.

| | Pos in | Risk | | | | Seq ID |
|---|---|---|---|---|---|---|
| SNP | NCBI B36 | Allele | D′ | r² | p-value | NO: |
| s.128352178 | 128352178 | G | 1 | 0.3 | 3.60E−10 | 882 |
| rs13262081 | 128353948 | G | 0.73 | 0.2 | 9.30E−05 | 883 |
| rs13280181 | 128355698 | A | 0.73 | 0.2 | 9.30E−05 | 309 |
| s.128367393 | 128367393 | T | 0.74 | 0.24 | 5.60E−05 | 884 |
| s.128368774 | 128368774 | T | 1 | 0.3 | 3.60E−10 | 885 |
| rs12679832 | 128370118 | C | 0.89 | 0.24 | 1.30E−05 | 886 |
| rs12707923 | 128370181 | C | 0.88 | 0.21 | 4.70E−05 | 310 |
| rs17378569 | 128371821 | T | 0.62 | 0.2 | 0.00012 | 887 |
| s.128373281 | 128373281 | G | 0.88 | 0.23 | 2.60E−05 | 888 |
| rs6984900 | 128373451 | T | 0.89 | 0.26 | 7.20E−06 | 311 |
| rs17450865 | 128376979 | T | 1 | 0.26 | 5.40E−09 | 312 |
| rs12549518 | 128378773 | A | 0.79 | 0.28 | 4.50E−06 | 314 |
| rs6996866 | 128379337 | T | 0.79 | 0.28 | 4.50E−06 | 315 |
| rs2007197 | 128380741 | T | 1 | 0.26 | 5.40E−09 | 316 |
| rs283727 | 128382542 | G | 0.93 | 0.4 | 3.50E−09 | 317 |
| rs283728 | 128382682 | T | 0.94 | 0.41 | 1.30E−09 | 318 |
| rs4871015 | 128383698 | G | 1 | 0.35 | 2.00E−13 | 889 |
| s.128384095 | 128384095 | A | 0.94 | 0.5 | 3.60E−11 | 890 |
| rs283704 | 128384764 | A | 1 | 0.41 | 4.40E−15 | 273 |
| rs56983490 | 128385858 | G | 0.72 | 0.22 | 2.60E−05 | 891 |
| rs283705 | 128386632 | C | 0.72 | 0.22 | 2.60E−05 | 274 |
| rs7006593 | 128386767 | T | 0.73 | 0.24 | 1.10E−05 | 892 |
| rs10107982 | 128387937 | T | 1 | 0.58 | 1.40E−18 | 321 |
| s.128390384 | 128390384 | T | 1 | 0.93 | 1.80E−30 | 893 |
| rs453875 | 128390593 | G | 0.88 | 0.69 | 1.30E−15 | 276 |
| rs445114 | 128392363 | T | 1 | 1 | 0.00E+00 | 3 |
| s.128393001 | 128393001 | C | 1 | 1 | 1.70E−34 | 894 |
| s.128393855 | 128393855 | A | 1 | 0.32 | 2.80E−12 | 895 |
| s.128394077 | 128394077 | T | 1 | 1 | 1.70E−34 | 896 |
| s.128397010 | 128397010 | A | 1 | 0.47 | 6.40E−17 | 897 |
| s.128399360 | 128399360 | T | 1 | 0.34 | 4.90E−13 | 898 |
| rs11785664 | 128399606 | C | 1 | 0.37 | 7.90E−14 | 278 |
| s.128399930 | 128399930 | A | 1 | 0.53 | 5.70E−17 | 899 |
| s.128400818 | 128400818 | C | 1 | 0.82 | 2.10E−26 | 900 |
| s.128400979 | 128400979 | A | 1 | 0.37 | 7.90E−14 | 901 |
| s.128401223 | 128401223 | G | 0.88 | 0.39 | 1.80E−08 | 452 |
| s.128401262 | 128401262 | A | 1 | 0.29 | 1.50E−11 | 902 |
| s.128401627 | 128401627 | G | 1 | 0.47 | 6.40E−17 | 903 |
| s.128401800 | 128401800 | G | 1 | 0.58 | 1.40E−18 | 904 |
| s.128402213 | 128402213 | G | 0.94 | 0.45 | 2.90E−10 | 905 |
| s.128402226 | 128402226 | T | 0.94 | 0.45 | 2.90E−10 | 906 |
| s.128402298 | 128402298 | A | 1 | 0.47 | 6.40E−17 | 907 |
| s.128402362 | 128402362 | C | 1 | 1 | 1.70E−34 | 908 |
| s.128402363 | 128402363 | G | 1 | 0.93 | 1.80E−30 | 909 |
| rs622556 | 128402379 | T | 1 | 0.47 | 6.40E−17 | 279 |
| rs452529 | 128402441 | G | 1 | 0.47 | 6.40E−17 | 280 |
| s.128402695 | 128402695 | A | 1 | 0.47 | 6.40E−17 | 910 |
| s.128403334 | 128403334 | T | 0.88 | 0.39 | 1.80E−08 | 456 |
| rs7832709 | 128403425 | A | 1 | 0.37 | 7.90E−14 | 911 |
| s.128403427 | 128403427 | A | 1 | 1 | 1.70E−34 | 912 |
| s.128403667 | 128403667 | T | 1 | 0.47 | 6.40E−17 | 913 |
| s.128403681 | 128403681 | T | 1 | 0.47 | 6.40E−17 | 914 |
| s.128403844 | 128403844 | A | 1 | 0.47 | 6.40E−17 | 915 |
| rs13256367 | 128404082 | A | 1 | 1 | 1.70E−34 | 327 |
| s.128404093 | 128404093 | G | 1 | 0.47 | 6.40E−17 | 916 |
| rs594076 | 128404148 | G | 1 | 0.47 | 6.40E−17 | 917 |
| s.128404174 | 128404174 | C | 0.88 | 0.39 | 1.80E−08 | 457 |
| rs437980 | 128404428 | A | 1 | 0.47 | 6.40E−17 | 918 |
| s.128404708 | 128404708 | C | 1 | 0.64 | 2.80E−20 | 919 |
| rs620861 | 128404855 | G | 1 | 1 | 1.70E−34 | 920 |
| rs620808 | 128404896 | C | 0.94 | 0.45 | 2.90E−10 | 921 |
| s.128404897 | 128404897 | A | 1 | 0.47 | 6.40E−17 | 922 |
| rs443053 | 128404978 | G | 1 | 1 | 1.70E−34 | 923 |
| s.128405181 | 128405181 | T | 1 | 0.28 | 1.40E−09 | 924 |
| rs11775799 | 128405418 | G | 1 | 0.37 | 7.90E−14 | 925 |
| rs400818 | 128405728 | T | 1 | 0.47 | 6.40E−17 | 281 |
| s.128405922 | 128405922 | A | 1 | 0.47 | 6.40E−17 | 926 |
| s.128405923 | 128405923 | C | 1 | 0.47 | 6.40E−17 | 927 |
| s.128405926 | 128405926 | G | 1 | 0.47 | 6.40E−17 | 928 |
| rs386883 | 128406053 | G | 1 | 0.47 | 6.40E−17 | 282 |
| rs377649 | 128406423 | G | 1 | 0.47 | 6.40E−17 | 283 |
| s.128406460 | 128406460 | C | 1 | 0.96 | 3.50E−32 | 929 |
| s.128407109 | 128407109 | A | 1 | 0.47 | 6.40E−17 | 930 |
| s.128407776 | 128407776 | G | 1 | 0.47 | 6.40E−17 | 931 |
| s.128407875 | 128407875 | G | 1 | 0.47 | 6.40E−17 | 932 |
| s.128407884 | 128407884 | C | 1 | 0.93 | 1.80E−30 | 933 |
| s.128407942 | 128407942 | G | 1 | 0.47 | 6.40E−17 | 934 |
| s.128408029 | 128408029 | G | 1 | 0.47 | 6.40E−17 | 935 |
| rs432470 | 128408226 | C | 1 | 0.39 | 1.20E−14 | 284 |
| rs424281 | 128408608 | G | 1 | 0.39 | 1.20E−14 | 285 |
| rs1668875 | 128410285 | G | 1 | 0.83 | 1.60E−27 | 288 |
| rs7002712 | 128410794 | T | 1 | 0.38 | 3.10E−14 | 289 |
| rs587948 | 128410862 | T | 0.92 | 0.75 | 1.30E−17 | 290 |
| rs623401 | 128410909 | C | 0.92 | 0.75 | 1.30E−17 | 291 |
| s.128411296 | 128411296 | C | 1 | 0.93 | 1.80E−30 | 936 |
| rs10956359 | 128411336 | T | 1 | 0.58 | 1.40E−18 | 341 |
| rs17464492 | 128412048 | A | 1 | 0.58 | 1.40E−18 | 342 |
| s.128412523 | 128412523 | G | 0.79 | 0.21 | 0.00012 | 937 |
| rs420101 | 128413061 | G | 0.9 | 0.57 | 3.40E−13 | 343 |
| rs7838714 | 128413130 | C | 1 | 0.34 | 4.90E−13 | 344 |
| rs389143 | 128413562 | C | 0.9 | 0.57 | 3.40E−13 | 345 |
| rs688201 | 128413584 | G | 0.9 | 0.57 | 3.40E−13 | 346 |
| s.128413592 | 128413592 | G | 0.9 | 0.57 | 3.40E−13 | 938 |
| rs687324 | 128413773 | T | 0.9 | 0.57 | 3.40E−13 | 347 |
| s.128413783 | 128413783 | T | 0.9 | 0.57 | 3.40E−13 | 939 |
| s.128413784 | 128413784 | G | 0.9 | 0.57 | 3.40E−13 | 940 |
| rs687279 | 128413806 | C | 0.6 | 0.32 | 4.40E−07 | 348 |
| rs436238 | 128414210 | C | 0.9 | 0.57 | 3.40E−13 | 349 |
| rs581761 | 128414413 | G | 0.9 | 0.57 | 3.40E−13 | 350 |
| rs673745 | 128414451 | C | 0.9 | 0.57 | 3.40E−13 | 351 |
| rs688937 | 128414563 | T | 0.9 | 0.57 | 3.40E−13 | 352 |
| rs672888 | 128414645 | A | 0.9 | 0.57 | 3.40E−13 | 353 |
| rs7826557 | 128414913 | A | 1 | 0.34 | 4.90E−13 | 354 |
| s.128415441 | 128415441 | A | 0.9 | 0.57 | 3.40E−13 | 941 |
| rs418269 | 128415540 | G | 0.9 | 0.57 | 3.40E−13 | 355 |
| s.128415799 | 128415799 | A | 0.75 | 0.33 | 6.30E−07 | 942 |
| rs385278 | 128416199 | T | 0.9 | 0.57 | 3.40E−13 | 356 |
| rs391640 | 128416306 | A | 0.75 | 0.33 | 6.30E−07 | 357 |
| rs670725 | 128416339 | A | 0.9 | 0.57 | 3.40E−13 | 358 |
| rs382824 | 128416906 | T | 0.91 | 0.7 | 7.50E−16 | 359 |
| rs383205 | 128417159 | G | 0.9 | 0.57 | 3.40E−13 | 360 |
| rs373616 | 128417244 | T | 0.9 | 0.57 | 3.40E−13 | 361 |
| rs400772 | 128417480 | G | 0.9 | 0.57 | 3.40E−13 | 943 |
| s.128418616 | 128418616 | C | 1 | 0.24 | 2.00E−08 | 944 |
| rs13275275 | 128418909 | A | 0.9 | 0.57 | 3.40E−13 | 362 |
| rs13248140 | 128419070 | G | 0.91 | 0.59 | 7.70E−14 | 363 |
| s.128419152 | 128419152 | G | 0.9 | 0.57 | 3.40E−13 | 945 |
| rs10956361 | 128419288 | G | 0.9 | 0.57 | 3.40E−13 | 364 |
| rs10956362 | 128419568 | A | 0.9 | 0.57 | 3.40E−13 | 365 |
| rs13249993 | 128419697 | G | 1 | 0.37 | 7.90E−14 | 366 |
| rs11777532 | 128419790 | C | 1 | 0.3 | 6.50E−12 | 367 |
| s.128420230 | 128420230 | A | 0.9 | 0.57 | 3.40E−13 | 946 |
| s.128420624 | 128420624 | A | 0.9 | 0.52 | 2.60E−12 | 947 |
| rs10956363 | 128420955 | G | 0.9 | 0.57 | 3.40E−13 | 368 |
| s.128421136 | 128421136 | C | 0.6 | 0.32 | 4.40E−07 | 948 |
| s.128421387 | 128421387 | T | 0.73 | 0.37 | 6.80E−08 | 949 |
| rs4871782 | 128421416 | G | 0.9 | 0.57 | 3.40E−13 | 369 |
| s.128421545 | 128421545 | G | 0.6 | 0.32 | 4.40E−07 | 950 |
| rs10087810 | 128421912 | T | 0.9 | 0.52 | 2.60E−12 | 370 |
| rs12541832 | 128422353 | C | 0.6 | 0.32 | 4.40E−07 | 371 |
| rs13262406 | 128422921 | A | 0.6 | 0.32 | 4.40E−07 | 372 |
| rs17465052 | 128423262 | A | 1 | 0.3 | 6.50E−12 | 951 |
| s.128423268 | 128423268 | A | 0.6 | 0.32 | 4.40E−07 | 952 |
| rs10098985 | 128424201 | T | 1 | 0.34 | 4.90E−13 | 373 |
| rs13281615 | 128424800 | A | 0.83 | 0.59 | 7.90E−13 | 374 |
| rs17465283 | 128425006 | A | 1 | 0.34 | 4.90E−13 | 953 |
| s.128425122 | 128425122 | A | 0.9 | 0.5 | 9.70E−12 | 954 |
| rs13256275 | 128425408 | G | 0.87 | 0.37 | 5.00E−08 | 295 |
| rs17465317 | 128425852 | C | 1 | 0.37 | 7.90E−14 | 955 |
| rs55746746 | 128426042 | T | 1 | 0.34 | 4.90E−13 | 956 |
| rs13267780 | 128426999 | G | 0.81 | 0.38 | 8.80E−08 | 376 |
| rs10447995 | 128427106 | G | 1 | 0.37 | 7.90E−14 | 377 |
| rs6999578 | 128427977 | T | 1 | 0.33 | 1.20E−12 | 957 |
| rs7014657 | 128430423 | G | 1 | 0.34 | 4.90E−13 | 378 |
| rs56110209 | 128431110 | G | 1 | 0.34 | 4.90E−13 | 958 |
| s.128432549 | 128432549 | G | 0.92 | 0.3 | 5.80E−07 | 959 |
| rs10097200 | 128432834 | C | 0.92 | 0.3 | 5.80E−07 | 960 |
| rs7002826 | 128433453 | C | 0.92 | 0.3 | 5.80E−07 | 379 |
| rs7007568 | 128434088 | C | 0.92 | 0.3 | 5.80E−07 | 380 |
| rs7842494 | 128435752 | A | 0.92 | 0.3 | 5.80E−07 | 381 |
| rs5022926 | 128436011 | C | 0.92 | 0.3 | 5.80E−07 | 382 |
| rs10112674 | 128436636 | T | 0.93 | 0.4 | 7.00E−09 | 961 |
| rs9693995 | 128437695 | T | 1 | 0.33 | 1.20E−12 | 383 |
| s.128439155 | 128439155 | G | 0.86 | 0.58 | 1.30E−12 | 962 |
| s.128439365 | 128439365 | G | 0.72 | 0.4 | 6.40E−09 | 963 |
| s.128439453 | 128439453 | G | 0.86 | 0.58 | 1.30E−12 | 964 |
| s.128439937 | 128439937 | G | 0.72 | 0.4 | 6.40E−09 | 965 |
| s.128440131 | 128440131 | C | 0.85 | 0.47 | 1.60E−10 | 966 |
| rs10096351 | 128441354 | A | 1 | 0.34 | 4.90E−13 | 967 |
| rs2121629 | 128442209 | T | 0.9 | 0.5 | 9.70E−12 | 384 |
| rs12541305 | 128442938 | C | 1 | 0.3 | 6.50E−12 | 968 |
| rs978683 | 128443299 | G | 0.86 | 0.58 | 1.30E−12 | 385 |
| rs9297753 | 128444452 | T | 1 | 0.34 | 4.90E−13 | 969 |
| rs9283954 | 128444552 | T | 1 | 0.32 | 2.80E−12 | 386 |
| rs7831303 | 128445914 | A | 1 | 0.33 | 1.20E−12 | 387 |
| rs7815100 | 128445983 | C | 0.92 | 0.3 | 5.80E−07 | 388 |
| rs7835046 | 128446108 | T | 0.69 | 0.35 | 1.60E−07 | 970 |
| rs4143118 | 128446650 | G | 0.85 | 0.47 | 1.60E−10 | 389 |
| rs6988647 | 128446838 | C | 0.92 | 0.3 | 5.80E−07 | 390 |
| rs7006882 | 128446849 | T | 0.85 | 0.47 | 1.60E−10 | 971 |
| rs9692890 | 128446956 | A | 1 | 0.34 | 4.90E−13 | 972 |
| rs9693143 | 128447207 | T | 0.92 | 0.3 | 5.80E−07 | 391 |
| rs28524866 | 128447373 | C | 0.85 | 0.47 | 1.60E−10 | 973 |
| rs10956364 | 128448065 | T | 0.85 | 0.49 | 4.50E−11 | 393 |
| rs11776330 | 128448145 | T | 0.85 | 0.49 | 4.50E−11 | 394 |
| rs7845452 | 128448591 | C | 1 | 0.34 | 4.90E−13 | 395 |
| rs16902126 | 128451539 | G | 0.84 | 0.26 | 8.10E−06 | 974 |
| rs7815245 | 128452779 | T | 0.92 | 0.3 | 5.80E−07 | 396 |
| rs1562430 | 128457034 | C | 0.92 | 0.3 | 5.80E−07 | 398 |
| rs2392780 | 128457207 | G | 0.92 | 0.3 | 5.80E−07 | 399 |
| rs7015780 | 128458689 | T | 1 | 0.32 | 2.80E−12 | 307 |
| s.128460594 | 128460594 | T | 0.75 | 0.34 | 2.10E−06 | 975 |
| s.128462414 | 128462414 | C | 0.65 | 0.4 | 3.20E−08 | 976 |
| s.128463670 | 128463670 | A | 0.54 | 0.23 | 0.00032 | 977 |
| s.128464690 | 128464690 | T | 0.64 | 0.28 | 4.20E−05 | 978 |

TABLE 20
Surrogate markers based on 1000 genome project
(http://www.1000genomes.org) to anchor marker rs8102476 on
Chromosome 19q13.2, with r² > 0.2 in Caucasians. Shown
is; Surrogate marker name, position of surrogate marker in NCBI
Build 36, the allele that is correlated with risk-allele of the anchor
marker, and D′, r², and P-values of the correlation between
the markers. Allelic codes are A = 1, C = 2, G = 3, T = 4.

| | Pos in | Risk | | | | Seq ID |
|---|---|---|---|---|---|---|
| SNP | NCBI B36 | Allele | D′ | r² | p-value | NO: |
| rs8108765 | 43169695 | A | 0.67 | 0.23 | 4.10E−05 | 764 |
| rs8110367 | 43170305 | T | 0.68 | 0.25 | 2.70E−05 | 401 |
| s.43174267 | 43174267 | G | 0.68 | 0.25 | 2.70E−05 | 765 |
| rs59255647 | 43182371 | T | 0.68 | 0.25 | 2.70E−05 | 766 |
| s.43183445 | 43183445 | T | 0.57 | 0.21 | 5.40E−05 | 767 |
| s.43183844 | 43183844 | A | 0.68 | 0.25 | 2.70E−05 | 768 |
| rs8113568 | 43185050 | T | 0.68 | 0.25 | 2.70E−05 | 769 |
| rs10500278 | 43186344 | G | 0.68 | 0.25 | 2.70E−05 | 402 |
| rs12460657 | 43187837 | C | 0.68 | 0.25 | 2.70E−05 | 770 |
| rs56321312 | 43189293 | C | 0.68 | 0.25 | 2.70E−05 | 771 |
| s.43191209 | 43191209 | A | 0.68 | 0.25 | 2.70E−05 | 772 |
| s.43191727 | 43191727 | T | 0.68 | 0.25 | 2.70E−05 | 773 |
| s.43205052 | 43205052 | G | 0.79 | 0.31 | 3.30E−06 | 774 |
| rs705503 | 43206158 | C | 0.55 | 0.22 | 5.70E−05 | 403 |
| rs1725516 | 43225848 | A | 0.63 | 0.2 | 0.00075 | 775 |
| rs1725517 | 43225864 | T | 0.63 | 0.2 | 0.00075 | 776 |
| rs1725518 | 43225875 | C | 0.63 | 0.2 | 0.00075 | 777 |
| rs1623976 | 43226317 | A | 0.63 | 0.2 | 0.00075 | 778 |
| rs1628394 | 43226848 | G | 0.63 | 0.2 | 0.00075 | 779 |
| rs7256656 | 43227905 | T | 0.63 | 0.2 | 0.00075 | 780 |
| rs1654338 | 43228193 | G | 0.63 | 0.2 | 0.00075 | 404 |
| s.43228365 | 43228365 | G | 0.63 | 0.2 | 0.00075 | 781 |
| rs6508759 | 43229065 | A | 0.63 | 0.2 | 0.00075 | 782 |
| rs1654339 | 43230639 | A | 0.63 | 0.2 | 0.00075 | 783 |
| rs1654340 | 43231546 | G | 0.63 | 0.2 | 0.00075 | 784 |
| rs1725459 | 43231572 | C | 0.63 | 0.2 | 0.00075 | 785 |
| rs734204 | 43231828 | A | 0.63 | 0.2 | 0.00075 | 786 |
| rs1725460 | 43232252 | T | 0.65 | 0.22 | 0.00028 | 787 |
| rs1616705 | 43233433 | A | 0.63 | 0.2 | 0.00075 | 788 |
| rs1725463 | 43233530 | T | 0.65 | 0.22 | 0.00028 | 789 |
| rs1620082 | 43233843 | A | 0.65 | 0.22 | 0.00028 | 790 |
| rs1725464 | 43234107 | A | 0.65 | 0.22 | 0.00028 | 791 |
| rs1618385 | 43234734 | C | 0.65 | 0.22 | 0.00028 | 792 |
| s.43234917 | 43234917 | G | 0.65 | 0.22 | 0.00028 | 793 |
| rs7249241 | 43235302 | T | 0.65 | 0.22 | 0.00028 | 794 |
| rs941036 | 43235335 | T | 0.65 | 0.22 | 0.00028 | 795 |
| rs941037 | 43235466 | C | 0.65 | 0.22 | 0.00028 | 796 |
| rs1725467 | 43235743 | A | 0.65 | 0.22 | 0.00028 | 797 |
| rs5022085 | 43236807 | A | 0.65 | 0.22 | 0.00028 | 798 |
| rs5022086 | 43236812 | A | 0.63 | 0.2 | 0.00075 | 799 |
| rs7256480 | 43236873 | C | 0.65 | 0.22 | 0.00028 | 800 |
| rs6508762 | 43236899 | C | 0.65 | 0.22 | 0.00028 | 801 |
| rs7256804 | 43236943 | G | 0.65 | 0.22 | 0.00028 | 802 |
| rs7256626 | 43237078 | A | 0.65 | 0.22 | 0.00028 | 803 |
| rs10421137 | 43237400 | C | 0.65 | 0.22 | 0.00028 | 804 |
| rs1654344 | 43237994 | C | 0.65 | 0.22 | 0.00028 | 805 |
| s.43237998 | 43237998 | C | 0.65 | 0.22 | 0.00028 | 806 |
| s.43318850 | 43318850 | G | 0.88 | 0.22 | 8.30E−05 | 807 |
| rs2005055 | 43318978 | A | 0.88 | 0.21 | 0.0001 | 808 |
| rs8103692 | 43416870 | C | 1 | 0.63 | 9.60E−22 | 809 |
| rs12610482 | 43417256 | T | 1 | 0.3 | 5.10E−11 | 810 |
| rs10409427 | 43417387 | A | 0.95 | 0.64 | 3.40E−14 | 811 |
| s.43417699 | 43417699 | T | 0.91 | 0.65 | 2.50E−14 | 812 |
| rs7253820 | 43417916 | A | 0.91 | 0.63 | 1.60E−13 | 813 |
| rs4803899 | 43419480 | A | 0.91 | 0.63 | 1.60E−13 | 405 |
| rs7246060 | 43423502 | G | 0.92 | 0.74 | 1.10E−16 | 407 |
| s.43425293 | 43425293 | G | 1 | 0.62 | 3.40E−21 | 814 |
| s.43425483 | 43425483 | G | 1 | 0.47 | 1.90E−16 | 815 |
| rs8102454 | 43427320 | G | 1 | 0.97 | 5.40E−34 | 816 |
| s.43427426 | 43427426 | G | 1 | 0.3 | 5.10E−11 | 817 |
| rs8102476 | 43427453 | C | 1 | | | 4 |
| s.43427644 | 43427644 | A | 1 | 0.9 | 4.90E−31 | 818 |
| s.43428325 | 43428325 | G | 1 | 0.28 | 1.50E−10 | 819 |
| s.43429472 | 43429472 | G | 1 | 0.21 | 3.00E−08 | 820 |
| s.43429970 | 43429970 | G | 1 | 0.56 | 4.80E−19 | 821 |
| s.43432176 | 43432176 | G | 1 | 0.21 | 3.00E−08 | 822 |
| s.43432765 | 43432765 | C | 1 | 1 | 3.60E−36 | 823 |
| s.43435211 | 43435211 | C | 1 | 0.21 | 3.00E−08 | 824 |
| rs12976534 | 43435802 | A | 0.96 | 0.86 | 1.20E−20 | 409 |
| s.43436225 | 43436225 | T | 1 | 0.21 | 3.00E−08 | 825 |
| rs12610267 | 43436573 | A | 1 | 0.9 | 4.90E−31 | 826 |
| s.43437877 | 43437877 | T | 1 | 0.22 | 1.10E−08 | 827 |
| rs4803934 | 43438407 | C | 1 | 0.87 | 8.30E−30 | 410 |
| rs11668070 | 43440753 | G | 1 | 0.93 | 2.10E−32 | 411 |
| s.43441169 | 43441169 | T | 1 | 0.33 | 5.10E−12 | 828 |
| s.43441174 | 43441174 | T | 1 | 0.33 | 5.10E−12 | 829 |
| s.43441940 | 43441940 | G | 1 | 0.93 | 2.10E−32 | 830 |
| s.43443415 | 43443415 | G | 1 | 0.28 | 1.50E−10 | 831 |
| rs7250689 | 43445465 | T | 1 | 0.9 | 4.90E−31 | 412 |
| s.43446079 | 43446079 | T | 1 | 0.81 | 1.20E−27 | 832 |
| rs3786872 | 43447929 | C | 1 | 0.33 | 5.10E−12 | 415 |
| rs58711382 | 43449980 | A | 0.59 | 0.22 | 0.0011 | 833 |
| rs3786877 | 43451020 | T | 0.82 | 0.5 | 1.10E−10 | 416 |
| s.43451244 | 43451244 | C | 1 | 0.33 | 5.10E−12 | 834 |
| s.43452410 | 43452410 | G | 1 | 0.51 | 1.00E−17 | 835 |
| s.43452458 | 43452458 | G | 1 | 0.31 | 1.10E−11 | 836 |
| rs10408768 | 43452532 | A | 0.69 | 0.26 | 1.40E−05 | 837 |
| s.43453573 | 43453573 | C | 0.86 | 0.54 | 1.10E−11 | 838 |
| s.43453577 | 43453577 | T | 1 | 0.22 | 1.10E−08 | 839 |
| rs10048529 | 43454845 | G | 0.82 | 0.5 | 1.10E−10 | 840 |
| rs8101725 | 43456912 | C | 0.82 | 0.5 | 1.10E−10 | 418 |
| s.43457500 | 43457500 | C | 0.82 | 0.5 | 1.10E−10 | 841 |
| s.43457897 | 43457897 | T | 0.75 | 0.29 | 1.50E−06 | 842 |
| s.43458212 | 43458212 | A | 1 | 0.22 | 1.10E−08 | 843 |
| s.43458950 | 43458950 | T | 0.82 | 0.5 | 1.10E−10 | 844 |
| s.43461834 | 43461834 | T | 0.82 | 0.5 | 1.10E−10 | 845 |
| rs12611009 | 43464321 | T | 0.82 | 0.5 | 1.10E−10 | 420 |
| rs3826896 | 43465362 | T | 0.82 | 0.5 | 1.10E−10 | 421 |
| rs2060243 | 43465806 | A | 0.82 | 0.5 | 1.10E−10 | 846 |
| s.43468987 | 43468987 | T | 0.81 | 0.48 | 2.70E−10 | 847 |
| s.43469211 | 43469211 | T | 0.82 | 0.5 | 1.10E−10 | 848 |
| rs8100926 | 43469277 | G | 0.82 | 0.5 | 1.10E−10 | 849 |
| s.43469566 | 43469566 | T | 0.82 | 0.5 | 1.10E−10 | 850 |
| s.43469919 | 43469919 | T | 0.82 | 0.5 | 1.10E−10 | 851 |
| s.43470755 | 43470755 | A | 0.55 | 0.28 | 4.60E−06 | 852 |
| rs1821284 | 43475421 | C | 0.75 | 0.29 | 1.50E−06 | 423 |
| rs4802324 | 43476934 | C | 0.75 | 0.29 | 1.50E−06 | 853 |
| s.43479805 | 43479805 | A | 0.75 | 0.29 | 1.50E−06 | 854 |
| s.43482475 | 43482475 | A | 0.75 | 0.29 | 1.50E−06 | 855 |
| rs4312417 | 43489029 | A | 0.7 | 0.2 | 7.60E−05 | 428 |
| rs3178327 | 43489926 | T | 0.7 | 0.2 | 7.60E−05 | 429 |
| rs3900981 | 43492005 | C | 0.9 | 0.24 | 3.60E−06 | 430 |
| s.43495536 | 43495536 | G | 0.9 | 0.24 | 3.60E−06 | 856 |
| rs11881305 | 43496058 | A | 0.7 | 0.2 | 7.60E−05 | 857 |
| s.43498612 | 43498612 | A | 0.7 | 0.2 | 7.60E−05 | 858 |
| rs3843754 | 43499024 | G | 0.7 | 0.2 | 7.60E−05 | 431 |
| s.43500344 | 43500344 | A | 0.7 | 0.2 | 7.60E−05 | 859 |
| s.43500345 | 43500345 | A | 0.7 | 0.2 | 7.60E−05 | 860 |
| s.43500352 | 43500352 | A | 0.76 | 0.23 | 1.50E−05 | 861 |
| s.43507057 | 43507057 | G | 0.65 | 0.22 | 0.00048 | 862 |
| s.43547186 | 43547186 | G | 0.9 | 0.25 | 5.00E−06 | 863 |
| rs1052375 | 43553173 | A | 0.63 | 0.37 | 5.30E−07 | 433 |
| s.43569837 | 43569837 | T | 0.79 | 0.3 | 7.90E−06 | 864 |
| s.43606416 | 43606416 | A | 1 | 0.22 | 9.30E−09 | 865 |
| rs4801752 | 43619801 | A | 0.53 | 0.23 | 9.90E−05 | 866 |
| s.43622138 | 43622138 | T | 0.53 | 0.23 | 9.90E−05 | 867 |
| rs892053 | 43624226 | C | 0.59 | 0.21 | 0.00015 | 868 |
| rs2229139 | 43627120 | G | 0.52 | 0.21 | 0.00016 | 869 |
| s.43627475 | 43627475 | A | 0.52 | 0.21 | 0.00016 | 870 |
| rs10407327 | 43627788 | T | 0.52 | 0.21 | 0.00016 | 871 |
| rs11083457 | 43628191 | G | 0.52 | 0.21 | 0.00016 | 872 |
| s.43628334 | 43628334 | G | 0.52 | 0.21 | 0.00016 | 873 |
| rs7249795 | 43628493 | C | 0.52 | 0.21 | 0.00016 | 874 |
| rs7254048 | 43628643 | A | 0.52 | 0.21 | 0.00016 | 875 |
| rs7253151 | 43628676 | T | 0.52 | 0.21 | 0.00016 | 876 |
| s.43633484 | 43633484 | G | 0.51 | 0.23 | 0.0001 | 877 |
| rs8104269 | 43637242 | G | 0.52 | 0.21 | 0.00016 | 878 |
| s.43640260 | 43640260 | G | 1 | 0.32 | 3.80E−12 | 879 |
| rs2304147 | 43642787 | T | 0.54 | 0.21 | 0.00026 | 880 |
| rs2304150 | 43647423 | G | 0.55 | 0.23 | 0.00017 | 437 |
| s.43729257 | 43729257 | C | 0.55 | 0.27 | 4.90E−05 | 881 |

1. A method of determining a susceptibility to prostate cancer, the method comprising:
analyzing nucleic acid sequence data from a human individual for at least one allele of at least one polymorphic marker selected from the group consisting of rs16902094, rs8102476, rs10934853 and rs445114, and markers in linkage disequilibirium therewith; wherein different alleles of the at least one polymorphic marker are associated with different susceptibilities to prostate cancer in humans, and
determining a susceptibility to prostate cancer from the nucleic acid sequence data.

2. The method of claim 1, further comprising:
obtaining the nucleic acid sequence data from a biological sample containing nucleic acid from the human individual, prior to the analyzing.

3. The method of claim 2, wherein the obtaining of the nucleic acid sequence data comprises a method that includes at least one procedure selected from amplifying nucleic acid from the biological sample; and performing a hybridization assay using a nucleic acid probe and nucleic acid from the biological sample, or from the amplifying.

4. A method of determining nucleic acid sequence data indicative of a susceptibility to prostate cancer, the method comprising:
analyzing nucleic acid from a human individual to obtain nucleic acid data for at least one allele of at least one polymorphic marker selected from the group consisting of rs16902094, rs8102476, rs10934853 and rs445114, and markers in linkage disequilibirium therewith; wherein different alleles of the at least one polymorphic marker are associated with different susceptibilities to prostate cancer in humans, and
preparing a report containing the nucleic acid sequence data for said at least one allele of the at least one polymorphic marker, wherein said report is written in a computer readable medium, printed on paper, or displayed on a visual display.

5. (canceled)

6. A method for determining a susceptibility to prostate cancer in a human individual, comprising:
determining the presence or absence of at least one allele of at least one polymorphic marker in a nucleic acid sample obtained from the individual, or in a genotype dataset from the individual, wherein the at least one polymorphic marker is selected from the group consisting of rs16902094, rs8102476, rs10934853 and rs445114, and markers in linkage disequilibrium therewith, and wherein determination of the presence of the at least one allele is indicative of a susceptibility to prostate cancer.

7. The method of claim 6, wherein the determining comprises analyzing nucleic acid in the sample using a method that includes at least one procedure selected from amplifying nucleic acid from the nucleic acid sample; and performing a hybridization assay using a nucleic acid probe and nucleic acid from the nucleic acid sample, or from the amplifying.

8. The method of claim 1, further comprising displaying the susceptibility to prostate cancer on a visual display selected from the group consisting of an electronic display and a printed report.

9. The method of claim 1, further comprising recording the susceptibility to prostate cancer on a computer readable medium.

10. (canceled)

11. (canceled)

12. The method of claim 1, comprising analyzing nucleic acid sequence data from the human individual for at least one allele of at least two of said polymorphic markers, wherein different haplotypes comprising alleles of the at least two polymorphic markers are associated with different susceptibilities to prostate cancer in humans.

13. The method of claim 4, comprising analyzing the nucleic acid to obtain nucleic acid sequence data for at least one allele of at least two of said polymorphic markers, wherein different haplotypes comprising alleles of the at least two polymorphic markers are associated with different susceptibilities to prostate cancer in humans.

14. The method of claim 6, comprising determining the presence or absence of at least one allele of at least two of said polymorphic markers in the nucleic acid sample, wherein different haplotypes comprising alleles of the at least two polymorphic markers are associated with different susceptibilities to prostate cancer in humans.

15. The method of claim 1, wherein determining of a susceptibility comprises comparing the nucleic acid sequence data to a database containing correlation data between the at least one polymorphic marker and susceptibility to prostate cancer.

16. The method of claim 15, wherein the database comprises at least one risk measure of susceptibility to prostate cancer for the at least one polymorphic marker.

17. The method of claim 15, wherein the database comprises a look-up table containing at least one risk measure of prostate cancer for the at least one polymorphic marker.

18. The method of claim 1, further comprising obtaining a biological sample from the human individual, and determining sequence of the at least one allele of the at least one polymorphic marker in nucleic acid from the sample.

19. The method of claim 1, wherein the nucleic acid sequence data is obtained from a preexisting record.

20. The method of claim 6, wherein the determining is based on a genotype dataset from a preexisting record.

21. The method of claim 1, wherein markers in linkage disequilibrium with rs8102476 are selected from the group consisting of the markers listed in Table 11 and Table 20.

22. The method of claim 21, wherein markers in linkage disequilibrium with rs8102476 are selected from the group consisting of the markers listed in Table 16.

23. The method of claim 1, wherein markers in linkage disequilibrium with rs10934853 are selected from the group consisting of the markers listed in Table 8 and Table 17.

24. The method of claim 23, wherein markers in linkage disequilibrium with rs10934853 are selected from the group consisting of the markers listed in Table 13.

25. The method of claim 1, wherein markers in linkage disequilibrium with rs16902094 are selected from the group consisting of the markers listed in Table 9 and Table 18.

26. The method of claim 25, wherein markers in linkage disequilibrium with rs16902094 are selected from the group consisting of the markers listed in Table 15.

27. The method of claim 1, wherein markers in linkage disequilibrium with rs445114 are selected from the group consisting of the markers listed in Table 10 and Table 19.

28. The method of claim 27, wherein markers in linkage disequilibrium with rs445114 are selected from the group consisting of the markers listed in Table 14.

29. The method of claim 1, wherein the at least one polymorphic marker is selected from the group consisting of rs16902094, rs8102476, rs10934853, rs445114, rs16902104, and rs620861.

30. The method of claim 1, wherein the susceptibility is increased susceptibility.

31. The method of claim 30, wherein the presence of the at least one allele or haplotype is indicative of increased susceptibility with a relative risk of at least 1.08.

32. The method of claim 29, wherein determination of the presence of allele G in rs16902094, allele C in rs8102476, allele A in rs10934853, allele T in rs445114, allele G in rs620861 or allele T in rs16902104 is indicative of increased susceptibility of prostate cancer.

33. The method of claim 30, further comprising administering to the human individual a standard of care therapeutic for prostate health.

34. The method of claim 1, further comprising reporting the susceptibility to at least one entity selected from the group consisting of the individual, a guardian of the individual, a genetic service provider, a physician, a medical organization, and a medical insurer.

35. The method of claim 1, wherein the individual is of an ancestry that includes Caucasian ancestry.

36. The method of claim 1, further comprising assessing the presence or absence of at least one additional genetic risk factor for prostate cancer in the individual.

37. The method of claim 36, wherein the additional genetic risk factor for prostate cancer is selected from the group consisting of rs2710646 allele A, rs2660753 allele T, rs401681 allele C, rs9364554 allele T, rs10486567 allele G, rs6465657 allele C, rs1447295 allele A, rs16901979 allele A, rs6983267 allele G, rs1571801 allele A, rs10993994 allele T, rs4962416 allele C, rs10896450 allele G, rs4430796 allele A, rs11649743 allele G, rs1859962 allele G, rs2735839 allele G, rs9623117 allele C, rs5945572 allele Ars7127900 allele A, rs10896449 allele G, rs8102476 allele C, rs5759167 allele G, rs10207654 allele A, rs7679673 allele C, rs1512268 allele A, rs10505483 allele A, and rs10086908 allele T.

38. A method of identification of a marker for use in assessing susceptibility to prostate cancer, the method comprising
a. identifying at least one polymorphic marker in linkage disequilibrium with at least one marker selected from the group consisting of rs16902094, rs8102476, rs10934853 and rs445114;
b. obtaining nucleic acid sequence data about a plurality of human individuals diagnosed with prostate cancer, and a plurality of control individuals, determining the presence or absence at least one allele of the at the least one polymorphic marker in the nucleic acid sequence data; and
c. determining the difference in frequency of the at least one allele between the individuals diagnosed with prostate cancer and the control group;
wherein determination of a significant difference in frequency of the at least one allele is indicative of the at least one marker being useful for assessing susceptibility to prostate cancer.

39. The method of claim 38, wherein an increase in frequency of the at least one allele in the at least one polymorphism in individuals diagnosed with prostate cancer, as compared with the frequency of the at least one allele in the control group is indicative of the at least one allele being useful for assessing increased susceptibility to prostate cancer.

40. The method of claim 38, wherein a decrease in frequency of the at least one allele in the at least one polymorphism in individuals diagnosed with prostate cancer, as compared with the frequency of the at least one allele in the control sample is indicative of the at least one allele being useful for assessing decreased susceptibility to, or protection against, prostate cancer.

41. The method of claim 38, further comprising reporting the susceptibility to prostate cancer for the marker in linkage disequilibrium on a visual display, or recording the susceptibility in a computer-readable medium or printed report.

42.-55. (canceled)

56. A computer-readable medium having computer executable instructions for determining susceptibility to prostate cancer, the computer readable medium comprising:
a. data identifying at least one allele of at least one polymorphic marker for at least one human subject;
b. a routine stored on the computer readable medium and adapted to be executed by a processor to determine risk of developing prostate cancer for the at least one polymorphic marker for the subject;
wherein the at least one polymorphic marker is selected from the group consisting of rs16902094, rs8102476, rs10934853 and rs445114, and markers in linkage disequilibrium therewith.

57. An apparatus for determining a genetic indicator for prostate cancer in a human individual, comprising:
a processor
a computer readable memory having computer executable instructions adapted to be executed on the processor to analyze marker and/or haplotype information for at least one human individual with respect to at least one polymorphic marker selected from the group consisting of rs16902094, rs8102476, rs10934853 and rs445114, and markers in linkage disequilibrium therewith, and generate an output based on the marker or haplotype information, wherein the output comprises a risk measure of the at least one marker or haplotype as a genetic indicator of prostate cancer for the human individual.

58. The apparatus according to claim 57, wherein the computer readable memory further comprises data indicative of the risk of developing prostate cancer associated with at least one allele of the at least one polymorphic marker or at least one haplotype, and wherein a risk measure for the human individual is based on a comparison of the at least one marker allele and/or haplotype status for the human individual to the risk associated with the at least one allele of the at least one polymorphic marker or the at least one haplotype.

59.-64. (canceled)

65. The method of claim 1, wherein linkage disequilibrium between markers is characterized by values of r² of at least 0.1.

66. (canceled)
